(19) United States
(12) Patent Application Publication
(10) Pub. No.: US 2025/0268854 A1
(43) Pub. Date: Aug. 28, 2025

(54) 2-AMINO-5,5-DIMETHYLHEXANOIC ACID DERIVATIVES AS SORTILIN MODULATORS FOR USE IN THE TREATMENT OF DISEASE OF THE CENTRAL NERVOUS SYSTEM

(71) Applicant: c/o Vesper Bio ApS, Copenhagen (DK)

(72) Inventors: Paul Brian LITTLE, Copenhagen (DK); Manuel Javier CASES-THOMAS, Copenhagen (DK); Mads Fuglsang KJØLBY, Copenhagen (DK); Anders NYKJÆR, Copenhagen (DK)

(21) Appl. No.: 18/688,974

(22) PCT Filed: Sep. 2, 2022
(86) PCT No.: PCT/EP2022/074536

§ 371 Date: Mar. 4, 2024

(30) Foreign Application Priority Data
Sep. 3, 2021 (EP) 21194937.5

(51) Int. Cl. A61K 31/198; A61K 31/366; A61K 31/404; A61K 31/4406; A61K 31/505; C07C 53/126; C07D 209/08; C07D 211/22; C07D 235/08; C07D 239/04; C07D 311/06

(52) U.S. Cl. CPC A61K 31/198; A61K 31/366; A61K 31/404; A61K 31/4406; A61K 31/505; C07C 53/126; C07D 209/08; C07D 211/22; C07D 235/08; C07D 239/04; C07D 311/06

(57) ABSTRACT

The present invention relates to a compound that binds to and modulates the activity of sortilin, wherein the compound has a blood-to-brain Kₚᵤᵤ greater than 0.1. The compounds are capable of crossing the blood brain barrier and are suitable for use in the treatment of a disease of the central nervous system. The invention also relates to compounds of formula (I), which are modulators of sortilin activity, pharmaceutical compositions comprising these compounds and the use of these compounds in the treatment or prevention of medical conditions where modulation of sortilin activity is beneficial.

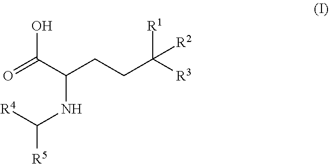

### RELATED APPLICATIONS

This application is a National Stage application of International Application No. PCT/EP2022/074536, filed Sep. 2, 2022 which claims priority under 35 U.S.C. § 119 to European Patent Application No. 21194937.5, filed Sep. 3, 2021.

### SEQUENCE LISTING

The Sequence Listing associated with this application is provided in XML format in lieu of paper copy, and is hereby incorporated by reference into the specification. The name of the XML file containing the Sequence Listing is “Sequence Listing ST26 220902 RMC00244WO.XML” The XML file is 7.40 KB, was created on Feb. 9, 2022, and was submitted electronically with the filing of the International Application.

### FIELD OF THE INVENTION

The invention relates to compounds that bind to and modulate the activity of sortilin and pharmaceutical compositions comprising these compounds. The invention also relates to their use in treating or preventing medical conditions where modulation of sortilin activity is beneficial. In particular, the invention relates to compounds that can cross the blood brain barrier and are useful in the treatment of diseases of the central nervous system.

### BACKGROUND

Sortilin is a Type I transmembrane protein that acts as a receptor of several ligands (Petersen et al., 1997). Sortilin is abundantly expressed in neurons and microglia of the nervous system, the inner ear, and in some peripheral tissues involved in metabolic control (Tauris et al., 2020; Goettsch et al., 2017; Willnow et al., 2011; Kjolby et al., 2010). Besides acting as a receptor involved in signaling, sortilin mediates sorting of select cargo between the cell surface trans-Golgi network, and endosomal pathway (Nykjaer & Willnow, 2012; Willnow, Petersen, & Nykjaer, 2008). Sortilin harbors a large extracellular domain denoted VPS10 that defines the family of receptors named sortilins or VS10p domain receptors. The VPS10P domain in sortilin is homologous to yeast VPS10P and is made up by a 10-bladed beta-propeller structure and a cysteine-rich 1000 module (Nykjaer & Willnow, 2012; Zheng, Brady, Meng, Mao, & Hu, 2011).

Sortilin binds multiple ligands, including pro-nerve growth factor (pro-NGF), pro-BDNF, pro-neurotrophin-3, neurotensin, and ApoB (Chen et al., 2005; Kjolby et al., 2010; Mazella et al., 1998; Nykjaer et al., 2004; Quistgaard et al., 2009; Yano, Torkin, Martin, Chao, & Teng, 2009). Furthermore, Sortilin binds progranulin (PGRN), a secreted protein involved in many cellular functions including securing lysosomal processes, anti-inflammatory responses, and neurotrophic stimulation (Galimberti, Fenoglio, & Scarpini, 2018). Sortilin targets PGRN for rapid endocytosis and degradation, and it is now well established that sortilin is the most important clearance receptor for PGRN (Hu et al., 2010). Thus, sortilin negatively regulates the extracellular levels of PGRN in the periphery as well as in the brain. Indeed, lack of or blocking the receptor increases plasma PGRN levels both in mice and humans (Carrasquillo et al., 2010; Gass, Prudencio, Stetler, & Petrucelli, 2012; Hu et al., 2010; Lee et al., 2014; Miyakawa et al., 2020; Pottier et al., 2018).

Frontotemporal dementia is a highly heritable dementia and haploinsufficiency of the PGRN gene accounts for up to 25% of all cases (Gijselinck, Van Broeckhoven, & Cruts, 2008). Patients with heterozygous loss-of-function mutations in PGRN have >50% reduced extracellular levels of the protein and will invariably develop FTD, making PGRN a causal gene for the disease (Baker et al., 2006; Carecchio et al., 2011; Cruts & Van Broeckhoven, 2008; Galimberti et al., 2010). In addition, PGRN mutant alleles have been identified in Alzheimer's (AD) patients (Brouwers et al., 2008; Sheng, Su, Xu, & Chen, 2014) and high levels of extracellular PGRN are protective in models of ALS, Parkinson's disease, stroke, arthritis, and atherosclerosis (Egashira et al., 2013; Laird et al., 2010; Martens et al., 2012; Tang et al., 2011; Tao, Ji, Wang, Liu, & Zhu, 2012; Van Kampen, Baranowski, & Kay, 2014).

Sortilin is, however, not required for PGRN to elicit its functions. Hence, neurons devoid in sortilin expression are equally responsive to PGRN-induced neuronal outgrowth (De Muynck et al., 2013; Gass, Lee, et al., 2012). Further, PGRN is successfully delivered to neuronal lysosomes in sortilin-deficient cells, suggesting the existence of alternative trafficking pathways. Indeed, PGRN can bind to the lysosomal protein, prosaposin (PSAP). When PSAP binds to its cognate receptors, the cation-independent mannose-6-phosphate receptor and LRP1, it brings along PGRN to the lysosomes (Zhou et al., 2015). Finally, in a phase II clinical trial with a monoclonal anti-sortilin antibody, markers for lysosomal integrity were normal (NCT03987295).

The functional PGRN receptor remains to be identified. However, studies suggest that PGRN promotes neuronal survival, reduces inflammation and increases Aβ endocytosis by microglia (Martens et al., 2012; Pickford et al., 2011; Yin et al., 2010).

Binding of PGRN to sortilin requires the three amino acids in the C-terminal of PGRN (QLL in human, PLL in mouse), and a peptide derived from the last 24 amino acids of PGRN binds with similar affinity as the full-length protein (Zheng et al., 2011). It was proposed that this mode of binding is structurally similar to Neurotensin binding (Zheng et al., 2011), i.e. binding in the NTIS1 binding site of sortilin. There has been a successful small molecule screen that identified a blocker of Neurotensin to sortilin binding done in collaboration with Aarhus University (Andersen et al., 2014; Schroder et al., 2014).

Sortilin exists as a full-length and sorting competent receptor but is also capable of forming multimeric signalling receptor-ligand. Portions of sortilin can also be liberated from the plasma membrane to scavenge ligands (NT in pain) and control the activity of ligands. For example, sortilin is involved in synaptic plasticity by controlling the conversion rate of pro-BDNF into BDNF. This may also apply to other proneurotrophins.

Finally, the propeptide of sortilin, also named spadin, that is a ligand of the receptor has been demonstrated to control the activity of the membrane transporter TREK-1, which is a target for major depression, among other diseases. Structurally, sortilin has an amino acid sequence according to SEQ ID NO: 1 and comprises a signal peptide, a propeptide, the Vps10p domain, a 10 cc domain (10CCa+10CCb), a transmembrane domain and a cytoplasmic tail. The luminal domain of sortilin has 6 potential N-linked glycosylation sites, whilst the cytoplasmic tail enables for the recruitment of various adapter proteins.

Sortilin binds to a vast number of ligands and membrane receptors and as a result engages in functions known to be important in cellular signalling and sorting. For example, sortilin is involved in signalling by proneurotrophins: the proforms of nerve growth factor (pro-NGF), brain derived neurotrophic factor (pro-BDNF), and neurotrophin-3 (proNT3), respectively. In complex with the protein p75NTR (p75 neurotrophin receptor), sortilin has been reported to form the receptor for proneurotrophin-mediated apoptotic effects leading to degeneration and cell death in cellular and animal models (Jansen et al., 2007; Tenk et al., 2005; Nykjaer et al., 2004).

Previous work has suggested a role for sortilin in cellular sorting and signalling associated with diseases such as diabetes and obesity (Huang et al., 2013). Sortilin facilitates translocation of GLUT4 to the plasma membrane and rescues it from degradation in the lysosomes (Pan et al., 2017). Sortilin levels have been shown to be modulated by the level of inflammation associated with these diseases. The pro-inflammatory cytokine, TNFα, reduces both mRNA levels and protein levels of sortilin in cultured mouse and human adipocytes, as well as in vivo when injected into mice (Kaddai et al., 2009). Sortilin can also influence cytokine secretion: targeting sortilin in immune cells has been proposed to attenuate inflammation and reduce atherosclerosis disease progression (Mortensen et al., 2014). Additionally, US 2016/0331746 describes various scaffolds of small molecules capable of binding to the active site of sortilin. Sortilin is involved in the regulation of glucose uptake (Shi & Kandror. 2005) and the development of lipid disorder diseases (Gao et al., 2017).

Further, plasma sortilin levels have been reported to be a potential biomarker for identifying patients with either coronary heart disease or diabetes mellitus (Oh et al., 2017; Møller et al., 2021). Patients that showed increased sortilin levels within their plasma, and therefore identifiable as suffering from the above conditions, also displayed enhanced glucose levels suggesting sortilin as a therapeutic target for treating these conditions. Soluble sortilin is also proposed as a treatment for type II diabetes (WO2021116290 A1, 2021).

TAR DNA-binding protein 43 (TDP-43) has been implicated in various neurodegenerative diseases. For example, TDP-43 inclusion bodies have been found in cases of amyotrophic lateral sclerosis (ALS), frontotemporal lobar degeneration (FTLD), and Alzheimer's disease (AD) (Meneses et al., 2021).

TDP-43 regulates the splicing of several gene products, including Sortilin. In humans, the splicing involves inclusion of a cryptic exon 17b (between exon 17 and 18), which introduces a stopcodon in the stalk, potentially generating a non-membrane bound fragment (Prudencio et al., 2012). Furthermore, it has been shown that PGRN can reduce levels of insoluble TDP-43 and slow down axonal degeneration (Beel et al., 2018). Sortilin inhibition increases PGRN levels and is therefore beneficial in the treatment of neurodegenerative diseases in which TDP 43 is implicated.

Sortilin has been linked to various conditions affecting the central nervous system (CNS). Some studies suggest a role of circulating sortilin in patients with psychiatric disorders such as depression, which may relate to altered activity of neurotrophic factors (Buttenshon et al., 2015); and it has also been reported that sortilin plays a role in brain aging, Alzheimer's Disease and frontotemporal dementia (Xu et al., 2019). However, delivering therapeutic agents that are capable of crossing the blood-brain barrier to the CNS presents a major challenge.

The blood-brain barrier is a highly selective semipermeable border of endothelial cells that prevents solutes in the circulating blood from non-selectively crossing into the extracellular fluid of the CNS where neurons reside. Therapy of neurological diseases is therefore limited due to the restricted penetration of therapeutic agents across the blood-brain barrier.

Therapeutic agents for treating CNS diseases therefore must be capable of crossing the blood-brain barrier. In addition to this, they must also have a sufficient unbound drug concentration in the brain, as the free drug hypothesis states that only unbound compound is able to interact with and elicit a pharmacological effect.

To determine the unbound fraction of a test compound (F_{ub}), sample supernatants may be analysed by methods such as liquid chromatography with tandem mass spectrometry (LC-MS/MS). The unbound fraction may then be calculated from the peak area ratios obtained for each matrix according to the following formula:

$${F}_{u b} = {C}_{P B S} / {C}_{p l a s m a}$$

where C_{PBS} and Cₚₗₐₛₘₐ are the analyte concentrations in PBS (receiver) and plasma (donor), respectively.

Recovery samples may be prepared in each condition but without dialysis, and may be used for evaluation of recovery from dialysis experiments using the following formula:

$$\% Recovery = 100 \times ( {V}_{P B S} \times {C}_{P B S} + {V}_{plasma} \times {C}_{plasma} ) / {V}_{plasma} \times {C}_{r e c o v e r y}$$

where V_{PBS} is the volume on the receiver side (PBS) and Vₚₗₐₛₘₐ is the volume on donor side (plasma) of the dialysis device. C_{recovery} is the analyte concentration measured from the recovery sample. Compounds such as propranolol or fluoxetine, may be included in experiments as controls.

The unbound fraction in brain (F_{ub, brain}) may be calculated from the measured value in brain homogenate (F_{ub, meas}), taking into account the dilution factor used in preparing the brain homogenate:

$${F}_{ub , brain} = \frac{1 / D}{( \frac{1}{{F}_{u b , m e a s}} ) - 1 + ( \frac{1}{D} )}$$

where D=dilution factor.

The brain/plasma unbound partition coefficient (Kₚᵤᵤ) may be determined as a ratio between the free compound concentrations in plasma and brain:

$${K}_{p u u} = \frac{{C}_{ub , brain}}{{C}_{ub , plasma}}$$

Where C_{u,brain}=unbound concentration in brain (C×F_{ub, brain}); wherein
C=concentration at steady state; and
C_{ub,plasma}=unbound concentration in plasma (C×F_{ub}).

For the treatment of CNS diseases, it is desirable for the Kₚᵤᵤ to have the highest possible value, above 0. A value around 1 indicating that the free fraction compound freely permeates the blood brain barrier; a value above 1 suggesting that an active influx transport mechanism at the blood brain barrier is involved; and less than 1, which indicates that the free fraction compound is either poorly permeable or is recognized by an active efflux mechanism, reducing the exposure in the CNS while passing back through the blood-brain barrier to plasma or the CSF. A Kₚᵤᵤ value of 0 or close to 0, indicates a poorly permeable compound or a highly active efflux mechanism that in any case will make highly improbable to reach a meaningful exposure in the CNS of the desired active species.

In view of the above, there is an unmet need for sortilin modulators that are capable of crossing the blood brain barrier and that have a Kₚᵤᵤ greater than 0.1 for use in the treatment of a disease of the central nervous system. The diseases of the central nervous system includes neurodegenerative disorders selected from motor neuron diseases, Frontotemporal Lobar Degeneration (FTLD), frontotemporal dementia, Alzheimer's disease, Parkinson's Disease, Huntington's disease, prion diseases such as Creutzfeldt-Jakob disease (CJD), acute brain injury, spinal cord injury and stroke; psychiatric disorders selected from bipolar disorder, major depression, post-traumatic stress disorder, and anxiety disorders; hearing loss selected from noise-induced hearing loss, ototoxicity induced hearing loss, age-induced hearing loss, idiopathic hearing loss, tinnitus and sudden hearing loss;
brain tumours (e.g. glioblastoma), retinopathies, glaucoma, neuroinflammation, chronic pain and diseases characterized by misfolded tau.

There is also an unmet need for new compounds that may be used in the treatment and prevention of medical conditions in which modulation of sortilin is beneficial, such as a neurodegenerative disorder, a psychiatric disorder, an inflammatory disorder, a cancer, pain, diabetes mellitus, diabetic retinopathy, glaucoma, uveitis, cardiovascular disease, kidney disease, psoriasis, hereditary eye conditions, hearing loss or diseases characterized by misfolded tau. The neurodegenerative disorder may be selected from motor neuron diseases, Frontotemporal Lobar Degeneration (FTLD), frontotemporal dementia, Alzheimer's disease, Parkinson's disease, Huntington's disease, prion diseases such as Creutzfeldt-Jakob disease (CJD), acute brain injury, spinal cord injury and stroke; the psychiatric disorder may be selected from bipolar disorder, major depression, post-traumatic stress disorder and anxiety disorders; the inflammatory disorder may be selected from inflammatory diseases and neuroinflammation; the cancer may be selected from breast cancer, lung cancer, ovarian cancer, prostate cancer, thyroid cancer, pancreatic cancer, glioblastoma and colorectal cancer; the cardiovascular disease may be selected from atherosclerosis, cardiomyopathy, heart attack, arrhythmias, heart failure, and ischemic heart disease; and the hearing loss may be selected from noise-induced hearing loss, ototoxicity induced hearing loss, age-induced hearing loss, idiopathic hearing loss, tinnitus and sudden hearing loss.

### SUMMARY OF THE INVENTION

The present invention relates to compounds that bind to and modulate the activity of sortilin, wherein the compound has a blood-to-brain Kₚᵤᵤ of more than 0.1. Such sortilin modulators can cross the blood brain barrier and can be used in the treatment or prevention of a disease of the central nervous system. The disease of the central nervous system may be selected from neurodegenerative disorders including motor neuron diseases, Frontotemporal Lobar Degeneration (FTLD), frontotemporal dementia, Alzheimer's disease, Parkinson's Disease, Huntington's disease, prion diseases such as Creutzfeldt-Jakob disease (CJD), acute brain injury, spinal cord injury, and stroke; psychiatric disorders including bipolar disorder, major depression, post-traumatic stress disorder, and anxiety disorders; hearing loss selected from noise-induced hearing loss, ototoxicity induced hearing loss, age-induced hearing loss, idiopathic hearing loss, tinnitus and sudden hearing loss; brain tumours (e.g. glioblastoma), retinopathies, glaucoma, neuroinflammation, chronic pain and diseases characterized by misfolded tau.

The invention also relates to compounds of formula (I), pharmaceutical compositions comprising these compounds and the use of these compounds in the treatment or prevention of medical conditions where modulation of sortilin activity is beneficial. Such medical conditions include a neurodegenerative disorder, a psychiatric disorder, an inflammatory disorder, a cancer, pain, diabetes mellitus, diabetic retinopathy, glaucoma, uveitis, cardiovascular disease, kidney disease, psoriasis, hereditary eye conditions, hearing loss or diseases characterized by misfolded tau.

### DETAILED DESCRIPTION

The inventors have surprisingly found that the compounds of the present invention are not only effective sortilin but are also able to cross the blood brain barrier. This is a property not previously seen with inhibitors of sortilin and provides opportunities for developing more effective therapies for diseases of the central nervous system.

Thus, in a first aspect, the present invention provides a compound that binds to and modulates the activity of sortilin, wherein the compound has a blood-to-brain Kₚᵤᵤ of more than 0.1.

The compounds are therefore capable of crossing the blood brain barrier and are suitable for use in the treatment of a disease of the central nervous system, as described herein.

The invention provides pharmaceutical compositions comprising a compound according to the first aspect and a pharmaceutically acceptable carrier, excipient and/or diluent.

The compounds or pharmaceutical compositions in accordance with the first aspect may be used in the treatment or prevention of a disease of the central nervous system. The disease may be selected from a neurodegenerative disorder selected from motor neuron diseases, Frontotemporal Lobar Degeneration (FTLD), frontotemporal dementia, Alzheimer's disease, Parkinson's disease, Huntington's disease, prion diseases such as Creutzfeldt-Jakob disease (CJD), acute brain injury, spinal cord injury and stroke; a psychiatric disorder selected from bipolar disorder, major depression, post-traumatic stress disorder, and anxiety disorders; hearing loss selected from noise-induced hearing loss, ototoxicity induced hearing loss, age-induced hearing loss, idiopathic hearing loss, tinnitus and sudden hearing loss; brain tumours, retinopathies, glaucoma, neuroinflammation, chronic pain and diseases characterized by misfolded tau.

The compounds may have a Kₚᵤᵤ of between 0.1 and 10, between 0.1 and 5, between 0.1 and 3, between 0.1 and 2, between 0.1 and 1, between 0.1 and 0.8, between 0.1 and 0.6, between 0.1 and 0.5, between 0.1 and 0.4, between 0.1 and 0.3, or between 0.1 and 0.2. In a second aspect, the present invention provides a compound of formula (1)

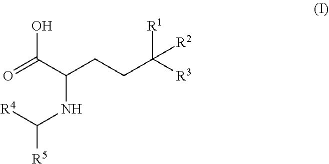

or a pharmaceutically acceptable salt, solvate, hydrate, tautomer, optical isomer, N-oxide, and/or prodrug thereof; wherein
R¹, R² and R³ are each independently selected from the group consisting of halo, H, (C₁-C₄)alkyl, halo-(C₁-C₄)alkyl, (C₂-C₄)alkenyl, and halo-(C₂-C₄)alkenyl; and
R⁴ is selected from the group consisting of H, (C₁-C₃)alkyl, halo-(C₁-C₃)alkyl, (C₃-C₈)aryl, halo-(C₃-C₈)aryl, (C₃-C₈)heteroaryl and halo-(C₃-C₈)heteroaryl;
R⁵ is selected from the group consisting of (C₃-C₂₀)-aryl, (C₃-C₂₀)-heteroaryl and 3- to 12-membered-heterocyclic ring;
   wherein the aryl, heteroaryl or heterocyclic ring is optionally substituted with one or more substituents independently selected from halo, —OH, cyano, carbonyl, (C₁-C₄)alkyl, (C₁-C₄)hydroxyalkyl, halo-(C₁-C₄)alkyl, acetyl, (C₁-C₄)alkoxy, halo-(C₁-C₄)alkoxy, (C₃-C₈)aryl and (C₃-C₈)heteroaryl; or
R⁴ and R⁵ taken together form a 6- to 20-membered-heterocyclic ring;
   wherein the heterocyclic ring is monocyclic, bicyclic or tricyclic and is optionally substituted with one or more substituents independently selected from halo, —OH, cyano, carbonyl, (C₁-C₄)alkyl, halo-(C₁-C₁₀)alkyl, acetyl, (C₁-C₄)alkoxy, and halo-(C₁-C₄)alkoxy.

It has been found that compounds of formula (1) according to the second aspect and sortilin modulators according to the first aspect bind and modulate sortilin and therefore may be useful in conditions where sortilin inhibition is beneficial. Such conditions include a neurodegenerative disorder, psychiatric diseases, an inflammatory disorder, a cancer, pain, diabetes mellitus, diabetic retinopathy, glaucoma, uveitis, cardiovascular disease, kidney disease, psoriasis, hereditary eye conditions, hearing loss or diseases characterized by misfolded tau. The neurodegenerative disorder may be selected from motor neuron diseases, Frontotemporal Lobar Degeneration (FTLD), frontotemporal dementia, Alzheimer's disease, Parkinson's disease, Huntington's disease, prion diseases such as Cretuzfeldt-Jakob disease (CJD), acute brain injury, spinal cord injury and stroke; the psychiatric disorder may be selected from bipolar disorder, major depression, post-traumatic stress disorder, and anxiety disorders; the inflammatory disorder may be selected from inflammatory diseases and neuroinflammation; the cancer may be selected from breast cancer, lung cancer, ovarian cancer, prostate cancer, thyroid cancer, pancreatic cancer, glioblastoma and colorectal cancer; the cardiovascular disease may be selected from atherosclerosis, cardiomyopathy, heart attack, arrhythmias, heart failure, and ischemic heart disease; and the hearing loss may be selected from noise-induced hearing loss, ototoxicity induced hearing loss, age-induced hearing loss, idiopathic hearing loss, tinnitus and sudden hearing loss.

As used herein, the term “sortilin” may refer to full length sortilin (also referred to as immature sortilin), comprising a signal peptide, a propeptide, a Vps10p domain, a 10 CC domain, a transmembrane domain and a large cytoplasmic tail, having an amino acid sequence according to SEQ ID NO: 1 or SEQ ID NO: 2, or it may refer to mature sortilin, comprising a Vps10p domain, a 10 CC domain, a transmembrane domain and a large cytoplasmic tail, having an amino acid sequence according to SEQ ID NO: 3, or a naturally occurring fragment, homologue or variant thereof. The term “sortilin” or “sortilin molecule” are used interchangeably herein. It is understood that sortilin is capable of interacting with a pro-neurotrophin molecule to form a sortilin/pro-neurotrophin complex. This sortilin/pro-neurotrophin complex may or may not be capable of interacting with a p75NTR molecule to form a trimeric complex comprising sortilin, pro-neurotrophin and p75NTR. It is understood that this trimeric complex may be responsible for adverse biological responses, such as stimulating apoptosis in retinal and ganglion cells, and controlling growth cone retraction of projecting axons (Jansen et al., 2007; Nykjaer et al., 2004; Santos et al., 2012; Skeldal et al., 2012).

As used herein, the term “pro-neurotrophin” refers to the larger precursors of neurotrophins, which undergo proteolytic cleavage to yield the mature form of the neurotrophin. Neurotrophins are a family of proteins that induce the survival, development and function of neurons, and are commonly referred to as growth factors. Pro-neurotrophins are biologically active and have distinct roles compared to their neurotrophin counterparts, such as induction of apoptosis. Examples of pro-neurotrophins include pro-NGF, pro-BDNF, proNT3 and proNT4. Pro-neurotrophins may also control synaptic plasticity. Whereas mature neurotrophins induce synaptic strength, in their proforms they may weaken synapses.

The compounds of the invention may be sortilin inhibitors, binders, modulators or antagonists. As used herein, the term “sortilin antagonist”, “sortilin inhibitor”, “sortilin binder” or “sortilin modulator” (used interchangeably) refers to a substance that interferes with, blocks, or otherwise attenuates the effect of, a sortilin protein binding to progranulin, or neurotensin or another extracellular ligand, or a pro-neurotrophin (e.g., pro-NGF, proNT3, pro-BDNF) or preventing the formation of the trimeric complex between sortilin, p75NTR and the pro-neurotrophin. The term “sortilin antagonist” also includes a substance or agent that interferes with the formation of a high affinity trimeric complex. In the latter scenario, it is recognised that a trimeric complex may be formed in that sortilin can bind to p75NTR (but not pro-NGF) and p75NTR can simultaneously bind the NGF domain of pro-NGF. However, the resulting trimeric complex may be of lower affinity for its receptor and as a result have significantly reduced capacity to stimulate apoptosis via the mechanism described above. Skeldal et al. (2012) demonstrated that the apoptotic function of the trimeric complex is abolished when sortilin is devoid in its intracellular domain. The term “sortilin antagonist” also includes a substance or agent that interferes with, blocks, or otherwise attenuates the effect of, a sortilin protein interacting with p75NTR. This interaction may be completely prevented, in which case the trimeric complex is prevented from forming, or only partially prevented, in which case the trimeric complex may be formed but may have reduced biological potency. Skeldal et al showed that complex formation between sortilin and p75NTR relies on contact points in the extracellular domains of the receptors and that the interaction critically depends on an extracellular juxtamembrane 23-amino acid sequence of p75NTR. Thus, the sortilin antagonist may interfere with this 23-amino acid sequence or proximal sequences in the molecules. “Sortilin antagonists” may act as ligand cellular uptake inhibitors wherein the ligands may be progranulin, neurotensin, BDNF etc.

It is preferred that R¹, R² and R³ are each independently selected from the group consisting of halo, (C₁-C₂)alkyl and halo-(C₁-C₂)alkyl.

In a preferred aspect of the invention, R¹, R² and R³ are each independently selected from F, CH₃ and CF₃. Most preferably, R¹, R² and R³ are the same. For example, in an exemplary compound of the invention, R¹, R² and R³ may each be F, CH₃ or CF₃.

In another preferred aspect of the invention, R⁴ is selected from the group consisting of H, (C₁-C₂)alkyl, halo-(C₁-C₂)alkyl, (C₅-C₈)aryl, halo-(C₅-C₈)aryl, (C₃-C₈)heteroaryl and halo-(C₃-C₈)heteroaryl.

The heteroaryl may comprise one, two or more heteroatoms. Preferably, the heteroaryl comprises one or two heteroatoms. The heteroatom may be selected from N, S or O. In groups with more than one heteroatom present, the heteroatoms may be the same or they may be different.

The aryl and heteroaryl groups may be monocyclic or bicyclic, preferably monocyclic. Preferably, the aryl and heteroaryl groups have between 5-8 carbon atoms. The heteroaryl group may have a ring size of 6-12 members, preferably 6-8 members.

In some preferred embodiments, R⁴ is selected from the group consisting of:
(i) H (ii) CH₃ (iii) CF₃
(iv) CHF₂ and

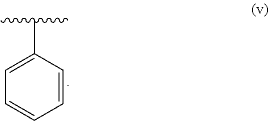 [SMILES: *c1ccccc1]

In another preferred aspect of the invention, R⁵ is wherein R⁵ is selected from the group consisting of (C₅-C₁₂)-aryl, (C₅-C₁₂)-heteroaryl and a 5- to 12-membered-heterocyclic ring; wherein the aryl, heteroaryl or heterocyclic ring is optionally substituted with one or more substituents independently selected from halo, —OH, cyano, carbonyl, (C₁-C₂)alkyl, (C₁-C₂)hydroxyalkyl, halo-(C₁-C₂)alkyl, (C₁-C₂)alkoxy, halo-(C₁-C₂)alkoxy (C₃-C₈)aryl and (C₃-C₈)heteroaryl.

The alkyl, haloalkyl, alkoxy and haloalkoxy substituents may be linear or branched.

The substituent may be attached at any position of the aryl, heteroaryl or heterocyclic ring. The one or more substituents may be attached to a carbon atom, heteroatom or combinations thereof. Preferably, there are no substituents or between one to five substituents.

The heteroaryl or heterocyclic ring may comprise one, two or more heteroatoms. Preferably, the heteroaryl or heterocyclic ring comprises one or two heteroatoms. The heteroatom may be selected from N, S or O. In groups with more than one heteroatom present, the heteroatoms may be the same or they may be different.

The heterocyclic ring may be aliphatic. It may be monocyclic, bicyclic or tricyclic. Preferably, the heterocyclic ring is monocyclic or bicyclic. Preferably, the heterocyclic ring has between 5-10 members, more preferably between 5-9 members.

The aryl and heteroaryl groups may also be monocyclic, bicyclic or tricyclic. Preferably, monocyclic or bicyclic. Preferably, the aryl and heteroaryl groups have a ring size of between 5-10 members.

Preferably, R⁵ is selected from the group consisting of:

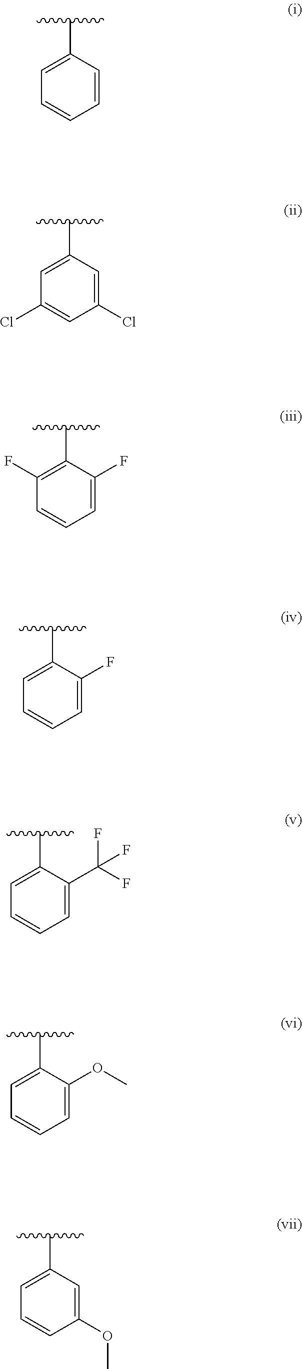

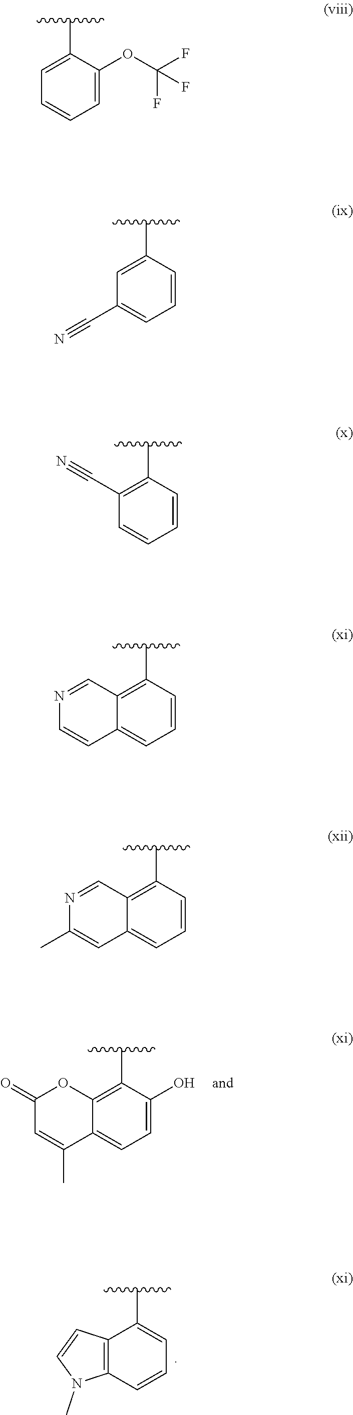

Alternatively, R⁴ and R⁵ taken together form an 8- to 20-membered-heterocyclic ring, wherein the heterocyclic ring is tricyclic.

Preferably, R⁴ and R⁵ taken together form the following structure:

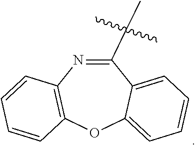 [SMILES: *C1=Nc2ccccc2Oc2ccccc21]

Particular compounds of the invention are those listed below.
(S)-2-(((7-hydroxy-4-methyl-2-oxo-2H-chromen-8-yl)methyl)amino)-5,5-dimethylhexanoic acid;
rac-2-(((7-hydroxy-4-methyl-2-oxo-2H-chromen-8-yl)methyl)amino)-5,5-dimethylhexanoic acid;
(S)-2-(benzylamino)-5,5-dimethylhexanoic acid;
(S)-5,5-dimethyl-2-(((1-methyl-1H-indol-4-yl)methyl)amino)hexanoic acid;
(S)-2-(benzhydrylamino)-5,5-dimethylhexanoic acid;
(S)-5,5-dimethyl-2-(((1-methyl-1H-indol-4-yl)methyl)amino)hexanoic acid;
(S)-5,5-dimethyl-2-(((R)-1-phenylethyl)amino)hexanoic acid;
(S)-5,5-dimethyl-2-(((S)-1-phenylethyl)amino)hexanoic acid;
(S)-5,5-dimethyl-2-(((S)-2,2,2-trifluoro-1-phenylethyl)amino)hexanoic acid;
(S)-5,5-dimethyl-2-(((R)-2,2,2-trifluoro-1-phenylethyl)amino)hexanoic acid;
(2S)-5,5-dimethyl-2-{[(3-methylisoquinolin-8-yl)methyl]amino}hexanoic acid;
(2S)-2-{[(3-methoxyphenyl)methyl]amino}-5,5-dimethylhexanoic acid;
(2S)-2-{[(isoquinolin-8-yl)methyl]amino}-5,5-dimethylhexanoic acid;
(2S)-5,5-dimethyl-2-({[2-(trifluoromethoxy)phenyl]methyl}amino)hexanoic acid;
(2S)-2-{[(2-fluorophenyl)methyl]amino}-5,5-dimethylhexanoic acid;
(2S)-2-{[(2,6-difluorophenyl)methyl]amino}-5,5-dimethylhexanoic acid;
(2S)-5,5-dimethyl-2-({[2-(trifluoromethyl)phenyl]methyl}amino)hexanoic acid;
(2S)-2-{[(1S)-2,2-difluoro-1-phenylethyl]amino}-5,5-dimethylhexanoic acid;
(2S)-2-{[(1R)-2,2-difluoro-1-phenylethyl]amino}-5,5-dimethylhexanoic acid;
(S)-2-(((S)-2,2-difluoro-1-(3-methoxyphenyl)ethyl)amino)-5,5-dimethylhexanoic acid;
(S)-2-(((R)-2,2-difluoro-1-(3-methoxyphenyl)ethyl)amino)-5,5-dimethylhexanoic acid;
(S)-5,5-dimethyl-2-(((R)-2,2,2-trifluoro-1-(3-methoxyphenyl)ethyl)amino)hexanoic acid:
(S)-5,5-dimethyl-2-(((S)-2,2,2-trifluoro-1-(3-methoxyphenyl)ethyl)amino)hexanoic acid;
(S)-2-((3-(hydroxymethyl)benzyl)amino)-5,5-dimethylhexanoic acid;
(S)-2-((2,3-dimethoxybenzyl)amino)-5,5-dimethylhexanoic acid;
(S)-2-((3,5-dimethoxybenzyl)amino)-5,5-dimethylhexanoic acid;
(S)-2-((2,5-dimethoxybenzyl)amino)-5,5-dimethylhexanoic acid;
(2S)-2-{[(3-fluoro-5-methoxyphenyl)methyl]amino}-5,5-dimethylhexanoic acid;
(2S)-2-{[(3-chloro-5-methoxyphenyl)methyl]amino}-5,5-dimethylhexanoic acid;
(2S)-2-{[(3-bromo-5-methoxyphenyl)methyl]amino}-5,5-dimethylhexanoic acid;
(2S)-2-{[(3,5-dichlorophenyl)methyl]amino}-5,5-dimethylhexanoic acid;
(2S)-2-{[(3-methoxy-4-methylphenyl)methyl]amino}-5,5-dimethylhexanoic acid;
(2S)-2-{[(2-fluoro-3-methoxyphenyl)methyl]amino}-5,5-dimethylhexanoic acid;
(2S)-5,5-dimethyl-2-{[(quinolin-3-yl)methyl]amino}hexanoic acid;
(2S)-5,5-dimethyl-2-{[(quinolin-2-yl)methyl]amino}hexanoic acid;
(2S)-2-{[(3-fluoro-4-methoxyphenyl)methyl]amino}-5,5-dimethylhexanoic acid;
(2S)-2-{[(3,4-dimethoxyphenyl)methyl]amino}-5,5-dimethylhexanoic acid;
(2S)-5,5-dimethyl-2-{[(5,6,7,8-tetrahydronaphthalen-1-yl)methyl]amino}hexanoic acid;
(2S)-2-{[(3,4-dihydro-2H-1-benzopyran-6-yl)methyl]amino}-5,5-dimethylhexanoic acid;
(2S)-2-{[(2,3-dihydro-1,4-benzodioxin-6-yl)methyl]amino}-5,5-dimethylhexanoic acid;
(2S)-5,5-dimethyl-2-{[(quinoxalin-6-yl)methyl]amino}hexanoic acid;
(2S)-5,5-dimethyl-2-[({1H-pyrrolo[2,3-b]pyridin-5-yl}methyl)amino]hexanoic acid;
(2S)-5,5-dimethyl-2-[({1H-pyrrolo[2,3-b]pyridin-4-yl}methyl)amino]hexanoic acid;
(2S)-2-{[(2H-1,3-benzodioxol-4-yl)methyl]amino}-5,5-dimethylhexanoic acid;
(2S)-5,5-dimethyl-2-{[(quinolin-6-yl)methyl]amino}hexanoic acid;
(2S)-5,5-dimethyl-2-{[(quinolin-8-yl)methyl]amino}hexanoic acid;
(2S)-5,5-dimethyl-2-{[(quinolin-5-yl)methyl]amino}hexanoic acid;
(2S)-2-{[(2-methoxynaphthalen-1-yl)methyl]amino}-5,5-dimethylhexanoic acid;
(2S)-2-{[(1H-indol-2-yl)methyl]amino}-5,5-dimethylhexanoic acid;
(2S)-2-{[(1,3-benzothiazol-5-yl)methyl]amino}-5,5-dimethylhexanoic acid;
(2S)-5,5-dimethyl-2-{[(1-methyl-1H-pyrazol-5-yl)methyl]amino}hexanoic acid;
(2S)-2-{[(1,3-benzothiazol-6-yl)methyl]amino}-5,5-dimethylhexanoic acid;
(2S)-5,5-dimethyl-2-{[(1-methyl-1H-indazol-6-yl)methyl]amino}hexanoic acid;
(2S)-5,5-dimethyl-2-{[(pyrimidin-5-yl)methyl]amino}hexanoic acid;
(2S)-5,5-dimethyl-2-({[2-(pyridin-4-yl)phenyl]methyl}amino)hexanoic acid;
(2S)-2-({[3-(1H-imidazol-1-yl)phenyl]methyl}amino)-5,5-dimethylhexanoic acid;
(2S)-5,5-dimethyl-2-{[(pyridin-4-yl)methyl]amino}hexanoic acid;
(2S)-2-({[2-(hydroxymethyl)phenyl]methyl}amino)-5,5-dimethylhexanoic acid;
(2S)-5,5-dimethyl-2-{[(1,5-naphthyridin-3-yl)methyl]amino}hexanoic acid;
(2S)-2-{[(1S)-1-(3,4-dimethoxyphenyl)-2,2-difluoroethyl]amino}-5,5-dimethylhexanoic acid;
(2S)-2-{[(1,7-dimethyl-1H-indol-4-yl)methyl]amino}-5,5-dimethylhexanoic acid;
(2S)-5,5-dimethyl-2-{[(1-methyl-1H-indazol-4-yl)methyl]amino}hexanoic acid;
(2S)-5,5-dimethyl-2-{[(1R)-1-(1-methyl-1H-indol-4-yl)ethyl]amino}hexanoic acid;
(2S)-2-{[(6-methoxynaphthalen-2-yl)methyl]amino}-5,5-dimethylhexanoic acid;
(2S)-2-{[(1S)-1-(3,4-dimethoxyphenyl)-2,2,2-trifluoroethyl]amino}-5,5-dimethylhexanoic acid;
(2S)-2-{[(1R)-1-(3,4-dimethoxyphenyl)-2,2-difluoroethyl]amino}-5,5-dimethylhexanoic acid;
(2S)-5,5-dimethyl-2-{[(1-methyl-1H-indol-7-yl)methyl]amino}hexanoic acid;
(2S)-2-{[(3,4-dimethylphenyl)methyl]amino}-5,5-dimethylhexanoic acid;
(2S)-2-{[(1S)-1-(4-methoxy-3-methylphenyl)ethyl]amino}-5,5-dimethylhexanoic acid;
(2S)-2-{[(1R)-1-(3,4-dimethoxyphenyl)-2,2,2-trifluoroethyl]amino}-5,5-dimethylhexanoic acid;
(2S)-5,5-dimethyl-2-{[(1-methyl-1H-1,3-benzodiazol-5-yl)methyl]amino}hexanoic acid;
(2S)-5,5-dimethyl-2-{[(1-methyl-1H-1,3-benzodiazol-4-yl)methyl]amino}hexanoic acid;
(2S)-2-{[(1S)-1-(3,4-dimethoxyphenyl)ethyl]amino}-5,5-dimethylhexanoic acid;
(2S)-5,5-dimethyl-2-{[(2-methylpyrimidin-5-yl)methyl]amino}hexanoic acid;
(2S)-5,5-dimethyl-2-{[(2-methyl-1,3-benzothiazol-5-yl)methyl]amino}hexanoic acid;
(2S)-2-{[(3-chloro-4-methylphenyl)methyl]amino}-5,5-dimethylhexanoic acid;
(2S)-2-{[(3-hydroxy-4-methoxyphenyl)methyl]amino}-5,5-dimethylhexanoic acid;
(2S)-5,5-dimethyl-2-{[(1-methyl-1H-1,2,3-benzotriazol-5-yl)methyl]amino}hexanoic acid;
(2S)-5,5-dimethyl-2-{[(pyrimidin-4-yl)methyl]amino}hexanoic acid;
(2S)-5,5-dimethyl-2-{[(1S)-1-(1-methyl-1H-indol-4-yl)ethyl]amino}hexanoic acid;
(2S)-2-{[(3-acetylphenyl)methyl]amino}-5,5-dimethylhexanoic acid;
(2S)-2-{[(1-ethyl-1H-indol-4-yl)methyl]amino}-5,5-dimethylhexanoic acid;
(2S)-2-{[(1-benzofuran-5-yl)methyl]amino}-5,5-dimethylhexanoic acid;
(2S)-2-{[(1S)-1-(2-methoxypyridin-4-yl)ethyl]amino}-5,5-dimethylhexanoic acid;
(2S)-2-{[(1R)-1-(4-methoxy-3-methylphenyl)ethyl]amino}-5,5-dimethylhexanoic acid;
(2S)-2-{[(3,4-dichlorophenyl)methyl]amino}-5,5-dimethylhexanoic acid;
(2S)-2-{[(5-bromopyridin-3-yl)methyl]amino}-5,5-dimethylhexanoic acid;
(2S)-5,5-dimethyl-2-[({1-methyl-1H-pyrrolo[2,3-b]pyridin-5-yl}methyl)amino]hexanoic acid;
(2S)-2-{[(5-methoxypyridin-3-yl)methyl]amino}-5,5-dimethylhexanoic acid;
(2S)-2-{[(1H-indol-4-yl)methyl]amino}-5,5-dimethylhexanoic acid;
(2S)-2-{[(isoquinolin-4-yl)methyl]amino}-5,5-dimethylhexanoic acid;
(2S)-5,5-dimethyl-2-{[(1R)-1-(pyrimidin-5-yl)ethyl]amino}hexanoic acid;
(2S)-2-{[(4-methoxyphenyl)methyl]amino}-5,5-dimethylhexanoic acid;
(2S)-5,5-dimethyl-2-{[(5-methylpyridin-3-yl)methyl]amino}hexanoic acid;
(2S)-2-{[(2,3-dimethylphenyl)methyl]amino}-5,5-dimethylhexanoic acid;
(2S)-5,5-dimethyl-2-{[(1S)-1-(pyrimidin-5-yl)ethyl]amino}hexanoic acid;
(2S)-2-{[(2H-indazol-4-yl)methyl]amino}-5,5-dimethylhexanoic acid;
(2S)-5,5-dimethyl-2-{[(6-methylpyridin-3-yl)methyl]amino}hexanoic acid;
(2S)-2-{[(2-chloro-3-fluoropyridin-4-yl)methyl]amino}-5,5-dimethylhexanoic acid;
(2S)-2-{[(isoquinolin-5-yl)methyl]amino}-5,5-dimethylhexanoic acid;
(2S)-2-{[(4-chlorophenyl)methyl]amino}-5,5-dimethylhexanoic acid;
(2S)-2-{[(2-methoxypyridin-4-yl)methyl]amino}-5,5-dimethylhexanoic acid;
(2S)-2-{[(2-fluoro-5-methoxyphenyl)methyl]amino}-5,5-dimethylhexanoic acid;
(2S)-5,5-dimethyl-2-{[(2-methylpyridin-4-yl)methyl]amino}hexanoic acid;
(2S)-5,5-dimethyl-2-{[(1-methyl-1H-indol-6-yl)methyl]amino}hexanoic acid;
(2S)-2-{[(1R)-1-(3,4-dimethoxyphenyl)ethyl]amino}-5,5-dimethylhexanoic acid;
(2S)-5,5-dimethyl-2-{[(3-methylpyridin-4-yl)methyl]amino}hexanoic acid;
(2S)-2-{[(3-methoxy-5-methylphenyl)methyl]amino}-5,5-dimethylhexanoic acid;
(2S)-2-{[(3-cyanophenyl)methyl]amino}-5,5-dimethylhexanoic acid;
(2S)-2-{[(4-methoxynaphthalen-1-yl)methyl]amino}-5,5-dimethylhexanoic acid;
(2S)-2-{[(4-fluoro-3-methoxyphenyl)methyl]amino}-5,5-dimethylhexanoic acid;
(2S)-5,5-dimethyl-2-{[(pyridin-3-yl)methyl]amino}hexanoic acid; and
(2S)-2-{[(3-methoxy-2-methylphenyl)methyl]amino}-5,5-dimethylhexanoic acid.

In preferred compounds of formula (1), R⁵ is selected from the group consisting of:
(i) phenyl, naphthyl, 5- or 6-membered monocyclic heteroaryl, and 9- or 10-membered fused bicyclic heteroaryl, each of which is optionally substituted with one or more substituents independently selected from the group consisting of halo, —OH, (C₁-C₂)alkyl, (C₁-C₂)alkoxy, (C₁-C₂)hydroxyalkyl, (C₁-C₂)haloalkyl, (C₁-C₂)haloalkoxy, acetyl, cyano, imidazolyl, and pyridyl; and

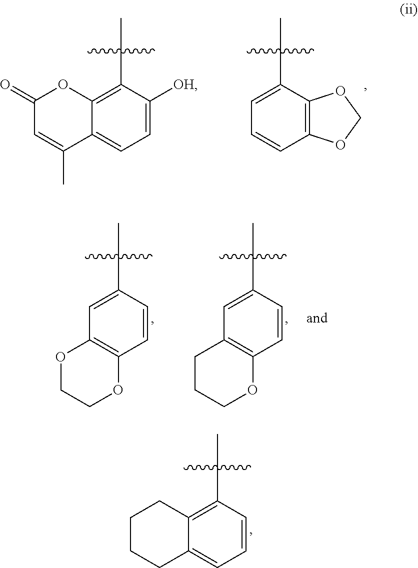 [SMILES: *c1c(O)ccc2c(C)cc(=O)oc12 ; *c1cccc2c1OCO2 ; *c1ccc2c(c1)OCCO2 ; *c1ccc2c(c1)CCCO2 ; *c1cccc2c1CCCC2]

The 5- or 6-membered monocyclic heteroaryl group contains 5 ring atoms, wherein no more than 2 ring atoms are a heteroatom (preferably N) and the other ring atoms are C.

The 9- or 10-membered fused bicyclic heteroaryl group contains two rings fused together such that they share two adjacent ring atoms. Preferably, the 9- or 10-membered fused bicyclic heteroaryl group contains a 6-membered ring fused to a 5- or 6-membered ring.

The 9- or 10-membered fused bicyclic heteroaryl group contains 9 or 10 ring atoms, wherein no more than 3 ring atoms are a heteroatom (preferably independently selected from N, O, and S) and the remaining ring atoms are C.

More preferably, R⁵ is selected from the group consisting of:
(i) phenyl, pyridyl, pyrimidinyl, pyrazolyl, quinolinyl, isoquinolinyl, indolyl, azaindolyl, quinoxalinyl, benzothiazolyl, indazolyl, naphthyridinyl, naphthyl, benzimidazolyl, benzotriazolyl, and benzofuranyl, each of which is optionally substituted with one or more substituents independently selected from the group consisting of halo, —OH, (C₁-C₂)alkyl, (C₁-C₂)alkoxy, (C₁-C₂)hydroxyalkyl, (C₁-C₂)haloalkyl, (C₁-C₂)haloalkoxy, acetyl, cyano, imidazolyl, and pyridyl; and

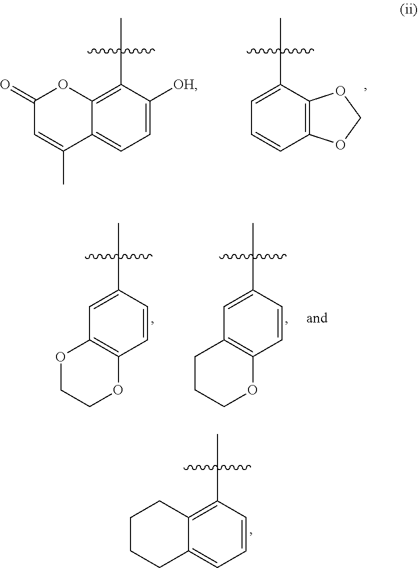 [SMILES: *c1c(O)ccc2c(C)cc(=O)oc12 ; *c1cccc2c1OCO2 ; *c1ccc2c(c1)OCCO2 ; *c1ccc2c(c1)CCCO2 ; *c1cccc2c1CCCC2]

More preferably, R⁵ is selected from the group consisting of:
(i) phenyl, optionally substituted with one or more substituents independently selected from the group consisting of halo, —OH, (C₁-C₂)alkyl, (C₁-C₂)alkoxy, (C₁-C₂)hydroxyalkyl, (C₁-C₂)haloalkyl, (C₁-C₂)haloalkoxy, acetyl, cyano, imidazolyl, and pyridyl;
(ii) pyridyl, optionally substituted with one or more substituents independently selected from the group consisting of halo, (C₁-C₂)alkyl, (C₁-C₂)alkoxy;
(iii) pyrimidinyl and pyrazolyl, each of which is optionally substituted with one or more substituents independently selected from the group consisting of (C₁-C₂)alkyl;
(iv) quinolinyl, isoquinolinyl, indolyl, azaindolyl, quinoxalinyl, benzothiazolyl, indazolyl, naphthyridinyl, naphthyl, benzimidazolyl, benzotriazolyl, and benzofuranyl, each of which is optionally substituted with one or more substituents selected from the group consisting of (C₁-C₂)alkyl and (C₁-C₂)alkoxy; and

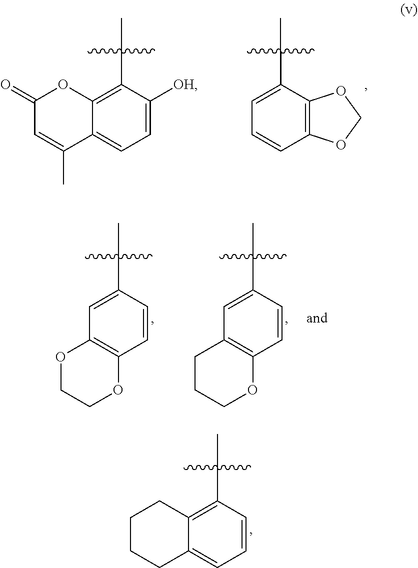 [SMILES: *c1c(O)ccc2c(C)cc(=O)oc12 ; *c1cccc2c1OCO2 ; *c1ccc2c(c1)OCCO2 ; *c1ccc2c(c1)CCCO2 ; *c1cccc2c1CCCC2]

More preferably, R⁵ is one of the following groups:

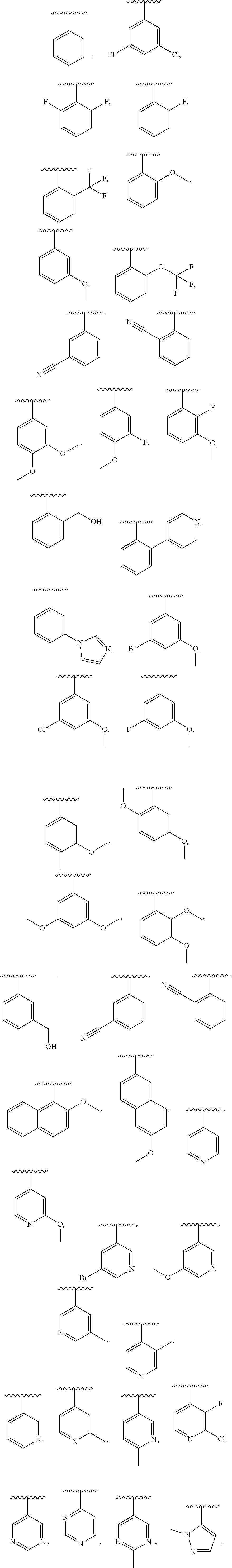

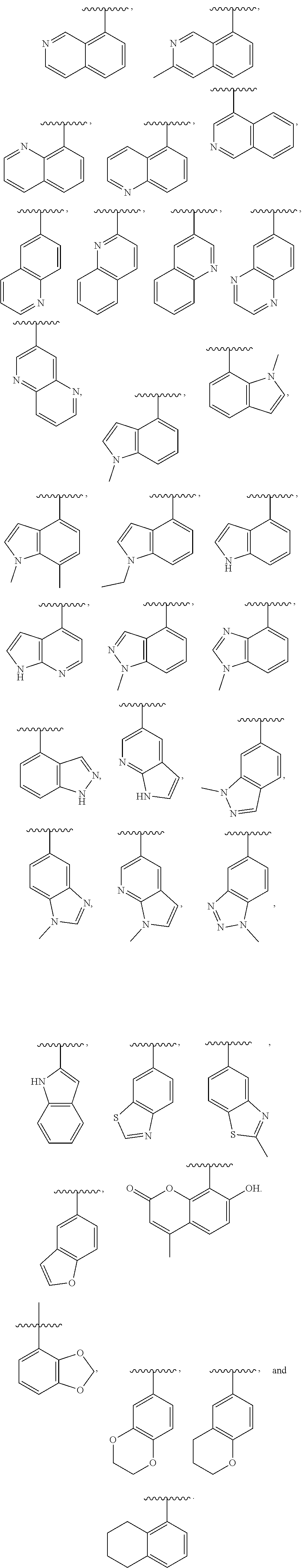

Alternatively, R⁴ and R⁵ are taken together to form the following group:

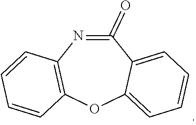

In the compounds of formula (I), it is highly preferred that R¹, R² and R³ are all CH₃.

Preferably, when R⁴ is phenyl in the compounds of formula (1), R⁵ is phenyl.

Preferably, the compounds of formula (1) have the following configuration.

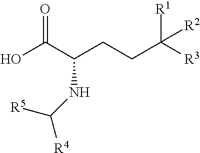

The compounds of formula (1) of the invention are intended for use in the treatment or prevention of a neurodegenerative disorder, a psychiatric disorder, an inflammatory disorder, a cancer, pain, diabetes mellitus, diabetic retinopathy, glaucoma, uveitis, cardiovascular disease, kidney disease, psoriasis, hereditary eye conditions, hearing loss or diseases characterized by misfolded tau. They may be used in the treatment or prevention of a disease of the central nervous system.

Preferably the neurodegenerative disorder is selected from motor neuron diseases, Frontotemporal Lobar Degeneration (FTLD), frontotemporal dementia, Alzheimer's disease, Parkinson's disease, Huntington's disease, prion diseases such as Creutzfeldt-Jakob disease (CJD), acute brain injury, spinal cord injury and stroke.

Preferably, the motor neuron disease is selected from amyotrophic lateral sclerosis (ALS), Primary Lateral Sclerosis, and Progressive Muscular Atrophy.

The neurodegenerative disorder is preferably a neurodegenerative disorder characterised by misfolded TAR DNA binding protein 43 (tdp-43). In other words, the neurodegenerative disease is characterized by truncated tdp-43 and inclusion bodies. Examples of such diseases include amyotrophic lateral sclerosis, Alzheimer's disease, Frontotemporal Lobar Degeneration, and frontotemporal dementia.

Preferably the psychiatric disorder is selected from bipolar disorder, major depression, post-traumatic stress disorder, and anxiety disorders.

Preferably the inflammatory disorder may be selected from inflammatory diseases and neuroinflammation.

Preferably the cancer is selected from breast cancer, lung cancer, ovarian cancer, prostate cancer, thyroid cancer, pancreatic cancer, glioblastoma and colorectal cancer.

Preferably, the cardiovascular disease is selected from atherosclerosis, cardiomyopathy, heart attack, arrhythmias, heart failure, and ischemic heart disease.

Preferably the hearing loss is selected from noise-induced hearing loss, ototoxicity induced hearing loss, age-induced hearing loss, idiopathic hearing loss, tinnitus and sudden hearing loss.

Thus, in an embodiment, the compounds for use according to the invention may disrupt interaction between a sortilin molecule and a pro-neurotrophin molecule, or disrupt the interaction between a sortilin molecule and a p75NTR molecule. Said sortilin molecule may be mature sortilin.

According to a third aspect of the invention, there is provided a pharmaceutical composition comprising a compound according to the first or second aspect of the invention and one or more pharmaceutically acceptable carriers, excipients, and/or diluents.

In a fourth aspect of the invention, there is provided a compound according to the first aspect of the invention, or a pharmaceutical composition according to the second aspect of the invention for use in therapy.

According to a fifth aspect of the invention, there is provided a compound according to the first or second aspect of the invention, or a pharmaceutical composition according to the third aspect of the invention for use in the treatment or prevention of a neurodegenerative disorder, a psychiatric disorder, an inflammatory disorder, a cancer, pain, diabetes mellitus, diabetic retinopathy, glaucoma, uveitis, cardiovascular disease, hereditary eye conditions or hearing loss.

Preferably, the neurodegenerative disorder is selected from motor neuron diseases, Frontotemporal Lobar Degeneration (FTLD), frontotemporal dementia, Alzheimer's disease, Parkinson's disease and spinal cord injury.

Preferably, the psychiatric disorder is selected from bipolar disorder, major depression, post-traumatic stress disorder, and anxiety disorders.

Preferably, the cancer is selected from breast cancer, lung cancer, ovarian cancer, prostate cancer, thyroid cancer, pancreatic cancer, glioblastoma, and colorectal cancer.

Preferably, the hearing loss is selected from noise-induced hearing loss, ototoxicity induced hearing loss, age-induced hearing loss, idiopathic hearing loss, tinnitus and sudden hearing loss.

Preferably the cardiovascular disease is selected from atherosclerosis, cardiomyopathy, heart attack, arrhythmias, heart failure, and ischemic heart disease (i.e. coronary artery disease).

According to a sixth aspect of the invention, there is provided the use of the compound according to the first or second aspect of the invention for the manufacture of a medicament for the treatment or prevention of a neurodegenerative disorder, a psychiatric disorder, an inflammatory disorder, a cancer, pain, diabetes mellitus, diabetic retinopathy, glaucoma, uveitis, cardiovascular disease, hereditary eye conditions or hearing loss.

According to a seventh aspect of the invention, there is provided a method for the treatment or prevention of a disease or condition responsive to sortilin modulation comprising administering a therapeutically effective amount of the compound according to the first or second aspect of the invention or the pharmaceutical composition according the third aspect of the invention.

The compounds of the invention may include isotopically-labelled and/or isotopically-enriched forms of the compounds. The compounds of the invention herein may contain unnatural proportions of atomic isotopes at one or more of the atoms that constitute such compounds. Examples of isotopes that can be incorporated into the disclosed compounds include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl.

The compounds of the invention may be used as such or, where appropriate, as pharmacologically acceptable salts (acid or base addition salts) thereof. The pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Compounds that have acidic properties can be converted to their pharmaceutically acceptable basic addition salts by treating the acid form with an appropriate base. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

Throughout the present disclosure, a given chemical formula or name shall also encompass all pharmaceutically acceptable salts, solvates, hydrates, N-oxides, and/or prodrug forms thereof. It is to be understood that the compounds of the invention include any and all hydrates and/or solvates of the compound formulas. It is appreciated that certain functional groups, such as the hydroxy, amino, and like groups form complexes and/or coordination compounds with water and/or various solvents, in the various physical forms of the compounds. Accordingly, the above formulas are to be understood to include and represent those various hydrates and/or solvates.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can be asymmetric (e.g. having one or more stereogenic centres). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis- and trans-geometric isomers of the compounds of the present invention are described and may be isolated as a mixture of isomers or as separated isomeric forms.

In the case of the compounds which contain an asymmetric carbon atom, the invention relates to the D form, the L form, and D,L mixtures and also, where more than one asymmetric carbon atom is present, to the diastereomeric forms. Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

The term “prodrugs” refers to compounds that may be converted under physiological conditions or by solvolysis to a biologically active compound of the invention. A prodrug may be inactive when administered to a subject in need thereof, but is converted in vivo to an active compound of the invention. Prodrugs are typically rapidly transformed in vivo to yield the parent compound of the invention, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2nd Ed., Elsevier Academic Press (2004), page 498 to 549). Prodrugs of a compound of the invention may be prepared by modifying functional groups, such as a hydroxy, amino or mercapto groups, present in a compound of the invention in such a way that the modifications are cleaved, either in routine manipulation or in vivo, to the parent compound of the invention. Examples of prodrugs include, but are not limited to, acetate, formate and succinate derivatives of hydroxy functional groups or phenyl carbamate derivatives of amino functional groups.

The term “treatment” as used herein may include prophylaxis of the named disorder or condition, or amelioration or elimination of the disorder once it has been established. The term “prevention” refers to prophylaxis of the named disorder or condition.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic imaging or sampling of subject tissue, fluids, specimens, cells, proteins, chemical markers, genetic materials, etc. as markers or indicators of the treatment regimen. In other methods, the subject is pre-screened or identified as in need of such treatment by assessment for a relevant marker or indicator of suitability for such treatment.

The invention provides a method of monitoring treatment progress. The method includes the step of determining a level of diagnostic marker (Marker) (e.g. any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

A level of Marker or Marker activity in a subject may be determined at least once. Comparison of Marker levels, e.g., to another measurement of Marker level obtained previously or subsequently from the same patient, another patient, or a normal subject, may be useful in determining whether therapy according to the invention is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radiolabelling/assay techniques, blotting/chemiluminescence methods, real-time PCR, and the like.

For clinical use, the compounds disclosed herein are formulated into pharmaceutical compositions (or formulations) for various modes of administration. It will be appreciated that compounds of the invention may be administered together with a physiologically acceptable carrier, excipient, and/or diluent (i.e. one, two, or all three of these). The pharmaceutical compositions disclosed herein may be administered by any suitable route, preferably by oral, rectal, nasal, topical (including ophthalmic, buccal and sublingual), sublingual, transdermal, intrathecal, transmucosal or parenteral (including subcutaneous, intramuscular, intravenous and intradermal) administration. Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration. The formulations can be further prepared by known methods such as granulation, compression, microencapsulation, spray coating, Etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, compounds disclosed herein may be incorporated into slow-release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

### Definitions

“Optional” or “optionally” means that the subsequently described event or circumstance may, but need not, occur, and that the description includes instances where the event or circumstance occurs and instances in which it does not.

The term “heteroatom” means O, N, or S.

The term “(C₁-Cₙ)alkyl” denotes a straight, branched or cyclic or partially cyclic alkyl group having from 1 to n carbon atoms, i.e. 1, 2, 3 . . . or n carbon atoms. For the “(C₁-Cₙ)alkyl” group to comprise a cyclic portion it should be formed of at least three carbon atoms. For parts of the range “(C₁-Cₙ)alkyl” all subgroups thereof are contemplated. For example, in the range (C₁-C₆)alkyl, all subgroups such as (C₁-C₅)alkyl, (C₁-C₄)alkyl, (C₁-C₃)alkyl, (C₁-C₂)alkyl, (C₁)alkyl, (C₂-C₆)alkyl, (C₂-C₅)alkyl, (C₂-C₄)alkyl, (C₂-C₃)alkyl, (C₂)alkyl, (C₃-C₆)alkyl, (C₃-C₅)alkyl, (C₃-C₄)alkyl, (C₃)alkyl, (C₄-C₆)alkyl, (C₄-C₅)alkyl, (C₄)alkyl, (C₅-C₆)alkyl, (C₆)alkyl. Examples of “C₁-C₆ alkyl” include methyl, ethyl, n-propyl, isopropyl, cyclopropyl, n-butyl, isobutyl, sec-butyl, t-butyl, cyclobutyl, cyclopropylmethyl, branched or cyclic or partially cyclic pentyl and hexyl Etc.

The term “halo-(C₁-Cₙ)alkyl” denotes a C₁-Cₙ alkyl as described above substituted with at least one halogen atom, which is preferably, F, Cl, Br and I, more preferably F and Cl, and most preferably F.

When a term denotes a range, for instance “1 to 6 carbon atoms” in the definition of (C₁-C₆)alkyl, each integer is considered to be disclosed, i.e. 1, 2, 3, 4, 5 and 6.

The term “(C₂-Cₙ)alkenyl” denotes a straight, branched or cyclic or partially cyclic alkyl group having at least one carbon-carbon double bond, and having from 2 to 6 carbon atoms. The alkenyl group may comprise a ring formed of 3 to 6 carbon atoms. For parts of the range “(C₂-Cₙ)alkenyl” all subgroups thereof are contemplated. For example, the range “(C₂-C₄)alkenyl” covers (C₂-C₄)alkenyl, (C₂-C₃)alkenyl, (C₂)alkenyl. Examples of “(C₂-C₄)alkenyl” include 2-propenyl, 2-butenyl, 3-butenyl, 2-methyl-2-propenyl Etc.

The term “(C₁-C₄)alkoxy” denotes —O—((C₁-C₄)alkyl) in which a (C₁-C₄)alkyl group is as defined above and is attached to the remainder of the compound through an oxygen atom. Examples of “(C₁-C₄)alkoxy” include methoxy, ethoxy, n-propoxy, isopropoxy, n-butoxy, isobutoxy, sec-butoxy and t-butoxy.

The term “halo(C₁-C₄)alkoxy” denotes a (C₁-C₄)alkoxy as described above substituted with a halogen atom, which is preferably, F, Cl, Br and I, more preferably F and Cl, and most preferably F.

The term “halo” means a halogen atom, and is preferably, F, Cl, Br and I, more preferably F and Cl, and most preferably F.

The term “3- to 12-membered heterocyclic ring” denotes a non-aromatic ring system having 3 to 12 ring atoms, in which at least one ring atoms is a heteroatom.

“An effective amount” refers to an amount of a compound of the invention that confers a therapeutic effect on the treated subject. The therapeutic effect may be objective (i.e. measurable by some test or marker) or subjective (i.e. subject gives an indication of or feels an effect).

As used herein, the terms “administration” or “administering” mean a route of administration for a compound disclosed herein. Exemplary routes of administration include, but are not limited to, oral, intraocular, intravenous, intraperitoneal, intraarterial, and intramuscular. The preferred route of administration can vary depending on various factors, e.g. the components of the pharmaceutical composition comprising a compound disclosed herein, site of the potential or actual disease and severity of disease.

The terms “subject” and “patient” are used herein interchangeably. They refer to a human or another mammal (e.g., mouse, rat, rabbit, dog, cat, cattle, swine, sheep, horse or primate) that can be afflicted with or is susceptible to a disease or disorder but may or may not have the disease or disorder. It is preferred that the subject is human.

Compounds of the invention may be disclosed by the name or chemical structure. If a discrepancy exists between the name of a compound and its associated chemical structure, then the chemical structure prevails.

The invention will now be further illustrated by the following non-limiting examples. The specific examples below are to be construed as merely illustrative, and not limitative of the remainder of the disclosure in any way whatsoever. Without further elaboration, it is believed that one skilled in the art can, based on the description herein, utilise the present invention to its fullest extent. All references and publications cited herein are hereby incorporated by reference in their entirety.

### Preparation of Compounds of the Invention

The compounds of the invention can be prepared according to the following General Synthetic Procedures scheme by methods well known and appreciated in the art. Suitable reaction conditions are well known in the art and appropriate substitutions of solvents and co-reagents are within the common general knowledge of the person skilled in the art. Likewise, it will be appreciated by those skilled in the art that synthetic intermediates may be isolated and/or purified by various well-known techniques as needed or desired, and that frequently, it will be possible to use various intermediates directly in subsequent synthetic steps with little or no purification. Furthermore, the skilled person will appreciate that in some circumstances, the orders in which moieties are introduced is not critical. The particular order of steps required to produce the compounds of formula (I) is dependent upon the particular compound being synthesized, the starting compound, and the relative liability of the substituted moieties as is well appreciated by those of ordinary skill in the art. All substituents, unless otherwise indicated, are as previously defined, and all reagents are well known and appreciated in the art.

Suitable starting materials, either optically active as single enantiomers or as racemic mixtures and protected amino acids of general formula AA-1 are either commercially available or may be prepared by a variety of methods. For example, as illustrated in the General Synthetic Procedure, Scheme 1, the carboxylic acid functionality of appropriately substituted amino acids of general formula AA-1, can be used as the free acid, PG=H, or protected as a suitable derivative, for example as a methyl ester. Insertion of the substituent on the primary amine present in AA-1 can be done by a variety of methods, and for the purpose of exemplification, by a reductive amination step involving a suitably substituted carbonyl compound Int-1, aldehyde (R⁵=H) or ketone (R⁴ and R⁵≠H) and a reductive reagent, as for example but not limited to, sodium triacetoxy borohydride in a suitable solvent mixture like acetic acid and dichloromethane. An alternative method to introduce the substituent on the primary amine present in AA-1, as represented in the general Synthetic Procedures scheme, uses an alkylation step between the suitable protected AA-1 and a reagent of type Int-2. In the later, LG represents a reactive leaving group, as for example, a bromine atom, that can be selectively displaced by the free amine in AA-1, in presence of a suitable base, like, for example, potassium carbonate, in an appropriate solvent like acetonitrile.

### General Synthetic Procedures

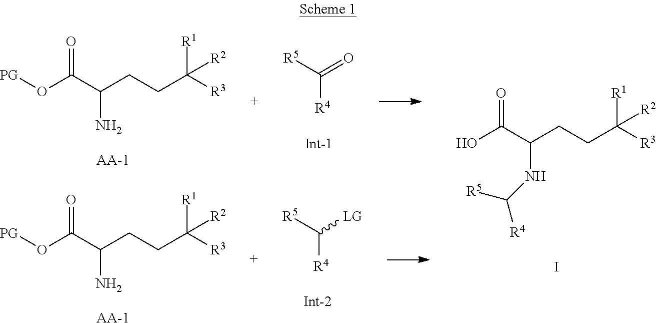

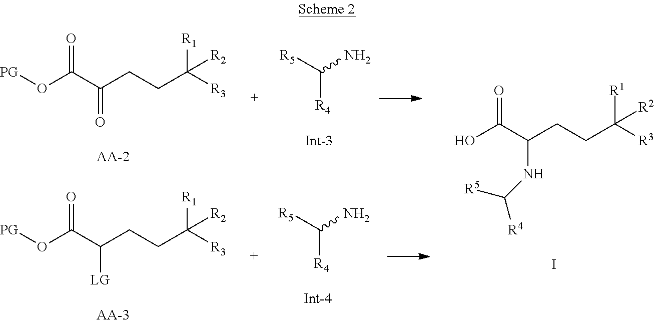

The compounds of general formula (1) may be prepared by a variety of procedures, some of which are described below. The products of each step can then be recovered by conventional methods including extraction, evaporation, precipitation, chromatography, filtration, trituration, crystallisation and the like.

Compounds of general formula (1) contain one or more stereogenic centres. Those can be introduced from available single enantiomers, optically active, starting materials of the type AA-1. The integrity of the existing stereogenic centre can be confirmed by analytical techniques well known to those skilled in the art like for example chiral support high pressure chromatography. Alternatively, when racemic starting materials are used, it will be appreciated that if desired, single isomer products can be obtained as single enantiomers or as single diastereoisomers, by known techniques like preparative chiral support high pressure chromatography.

The skilled artisan will also appreciate that not all of the substituents in the compounds of formula (1) will tolerate certain reaction conditions employed to synthesise the compounds. These moieties may be introduced at a convenient point in the synthesis, or may be protected and then deprotected as necessary or desired, as is well known in the art. The skilled artisan will appreciate that the protecting groups may be removed at any convenient point in the synthesis of the compounds of the present invention. Methods for introducing or removing protecting groups used in this invention are well known in the art; see, for example, Greene and Wuts, Protective Groups in Organic Synthesis, 4th Ed., John Wiley and Sons, New York (2006).

### EXAMPLES

### Abbreviations

approx: approximately; aq: aqueous; br: broad; ca.: circa; CDI: 1,1′-Carbonyldiimidazole; d: doublet; DCM: dichloromethane; DIC: N,N′-Diisopropylcarbodiimide; dioxane: 1,4-dioxane; DIPEA: diisopropylethylamine; DMF: dimethylformamide; eq.: equivalent; Et₃N: triethylamine; EtOAc: ethyl acetate; EtOH: ethanol; Fmoc: fluorenylmethoxycarbonyl; Boc: tert-butoxycarbonyl; h: hours; min: minutes: HATU: 2-(3H-[1,2,3]triazolo[4,5-b]pyridin-3-yl)-1,1,3,3-tetramethyl isouronium hexafluorophosphate(V); HPLC: high performance liquid chromatography; IPA, isopropanol; LC: liquid chromatography; m: multiplet; M: molar, molecular ion; MeCN: acetonitrile; MeOH: methanol; MS: mass spectrometry; NMR: nuclear magnetic resonance; PDA: photodiode array; q: quartet; rt: room temperature (ca. 20° C.); R_{T}: retention time; s: singlet, solid; SPPS: solid phase peptide synthesis. t: triplet; TBAF: tetrabutylammonium fluoride; TBME: tert-butyl methyl ether; TFA: trifluoroacetic acid; THF: tetrahydrofuran; UPLC: ultra-performance liquid chromatography; UV: ultraviolet.

Other abbreviations are intended to convey their generally accepted meaning.

### General Experimental Conditions

All starting materials and solvents were obtained either from commercial sources or prepared according to the literature citation. Reaction mixtures were magnetically stirred, and reactions performed at room temperature (ca. 20° C.) unless otherwise indicated.

Column chromatography was performed on an automated flash chromatography system, such as a CombiFlash Rf system, using pre-packed silica (40 μm) cartridges, unless otherwise indicated.

¹H-NMR spectra were recorded at 400 MHz on a Bruker Avance AV-1-400 or on a Bruker Avance AV-II-400 instrument. Chemical shift values are expressed in ppm-values relative to tetramethylsilane unless noted otherwise. The following abbreviations or their combinations are used for multiplicity of NMR signals: br=broad, d=doublet, m=multiplet, q=quartet, quint=quintet, s=singlet and t=triplet.

### Analytical Methods

Method 1—UPLC_AN_BASE, Apparatus: Waters IClass; Bin. Pump: UPIBSM, SM: UPISMFTN with SO; UPCMA, PDA: UPPDATC, 210-320 nm, SQD: ACQ-SQD2 ESI; ELSD: gaspressure 40 psi, drift tube temp: 50° C.; column: Waters XSelect CSH C18, 50×2.1 mm, 2.5 μm, Temp: 25° C., Flow: 0.6 mL/min, Gradient: t0=5% B, t2.0 min=98% B, t2.7 min=98% B, Posttime: 0.3 min, Eluent A: 10 mM ammonium bicarbonate in water (pH=9.5), Eluent B: acetonitrile.

Method 2—PREP_ACID-AS4A, Apparatus: Agilent Technologies G6130B Quadrupole; HPLC instrument type: Agilent Technologies 1290 preparative LC; Column: Waters XSelect CSH (C18, 100×30 mm, 10μ); Flow: 55 mL/min; Column temp: RT; Eluent A: 0.1% formid acid in water; Eluent B: 100% acetonitrile lin. gradient: t=0 min 20% B, t=2 min 20% B, t=8.5 min 60% B, t=10 min 100% B, t=13 min 100% B; Detection: DAD (220-320 nm); Detection: MSD (ESI pos/neg) mass range: 100-1000; fraction collection based on MS and DAD.

Method 3—UPLC Acidic Method, Apparatus: Waters HClass; Binary Solvent Pump, SM-FTN, CMA, PDA, QDa; Column: Waters ACQUITY UPLC® CSH (C18, 1.7 μm, 2.1×30 mm at 40° C.); Detection: UV at 210-400 nm unless otherwise indicated, MS by electrospray ionization; Solvents: A: 0.1% Formic in water, B: MeCN Gradient:

| Time | % A | % B | Flow rate (ml/min) |
|---|---|---|---|
| 0.00 | 98 | 2 | 0.77 |
| 2.50 | 0 | 100 | 0.77 |
| 3.00 | 0 | 100 | 0.77 |

Method 4—UPLC Basic Method; Apparatus: Waters HClass; Binary Solvent Pump, SM-FTN, CMA, PDA, QDa; Column: Waters ACQUITY UPLC® BEH (C18, 1.7 μm, 2.1×30 mm at 40° C.); Detection: UV at 210-400 nm unless otherwise indicated, MS by electrospray ionization; Solvents: A: 0.2% Ammonia in water, B: MeCN. Gradient:

| Time | % A | % B | Flow rate (ml/min) |
|---|---|---|---|
| 0.00 | 98 | 2 | 0.77 |
| 2.50 | 0 | 100 | 0.77 |
| 3.00 | 0 | 100 | 0.77 |

Method 5—#acid3minb; Apparatus: Agilent 1260; Quaternery Pump, HiP Sampler, Column Compartment, DAD: G6150 MSD; Column: Waters Cortecs (C18, 30×2.1 mm, 2.7 μm, at 40° C.); Detection: UV at 260 nm+/−90 nm unless otherwise indicated, MS by electrospray ionization; Solvents: A: 0.1% formic acid in water, B: MeCN. Gradient:

| Time | % A | % B | Flow rate (ml/min) |
|---|---|---|---|
| 0.00 | 98 | 2 | 1.35 |
| 2.50 | 0 | 100 | 1.35 |
| 3.00 | 0 | 100 | 1.35 |

Method 6—#basic3minb; Apparatus: Agilent 1260; Quaternery Pump, HiP Sampler, Column Compartment, DAD: G6150 MSD; Column: Phenomenex Evo (C18, 30×2.1 mm, 2.6 μm, at 40° C.); Detection: UV at 260 nm+/−90 nm unless otherwise indicated, MS by electrospray ionization; Solvents: A: 0.2% Ammonia in water, B: MeCN; Gradient:

| Time | % A | % B | Flow rate (ml/min) |
|---|---|---|---|
| 0.00 | 98 | 2 | 1.35 |
| 2.50 | 0 | 100 | 1.35 |
| 3.00 | 0 | 100 | 1.35 |

### Example 1

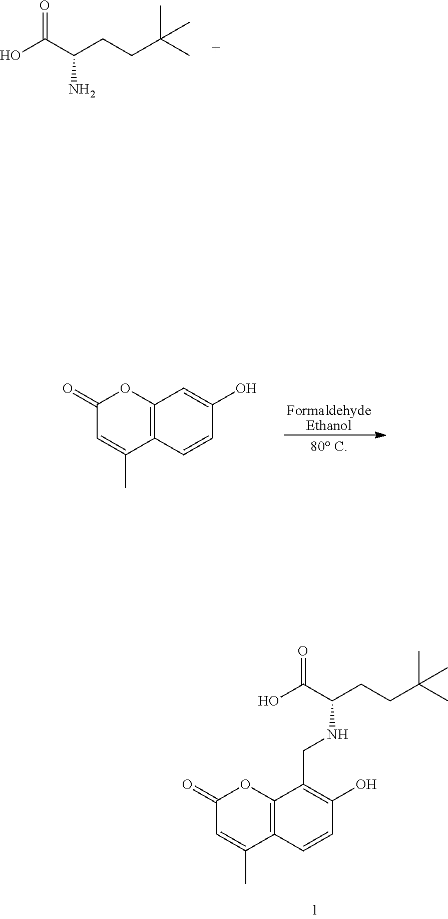 [SMILES: CC(C)(C)CC[C@H](N)C(=O)O ; Cc1cc(=O)oc2cc(O)ccc12 ; "80° C." ; "FormaldehydeEthanol" ; Cc1cc(=O)oc2c(CN[C@@H](CCC(C)(C)C)C(=O)O)c(O)ccc12]

### Synthesis of (S)-2-(((7-hydroxy-4-methyl-2-oxo-2H-chromen-8-yl)methyl)amino)-5,5-dimethylhexanoic acid 1

(S)-2-amino-5,5-dimethylhexanoic acid hydrochloride (100 mg, 0.51 mmol) and formaldehyde (0.084 mL, 3.07 mmol, 6 equiv.) were added to a solution of 7-hydroxy-4-methylcoumarin (90 mg, 0.51 mmol, 1 equiv.) in ethanol (2 mL). The mixture was stirred at 80° C. for 16 hours. After cooling down to room temperature the white precipitate was collected by filtration the filter was rinsed with ethanol (5 mL) and (S)-2-(((7-hydroxy-4-methyl-2-oxo-2H-chromen-8-yl)methyl)amino)-5,5-dimethylhexanoic acid (45 mg, 0.130 mmol, 25 yield, 96.96% purity) was isolated as white powder. LCMS (Method 1, 0.817 min; M+H=348.2; calcd. 348.2).

¹H-NMR (400 MHz, DMSO) δ 7.54 (t, J=6.6 Hz, 1H), 6.86-6.71 (m, 1H), 6.12 (s, 1H), 4.05 (d, J=6.3 Hz, 2H), 3.17 (t, J=6.1 Hz, 1H), 2.38 (s, 3H), 1.75-1.46 (m, 2H), 1.27-1.18 (m, 2H), 0.84 (s, 9H).

### Example 2

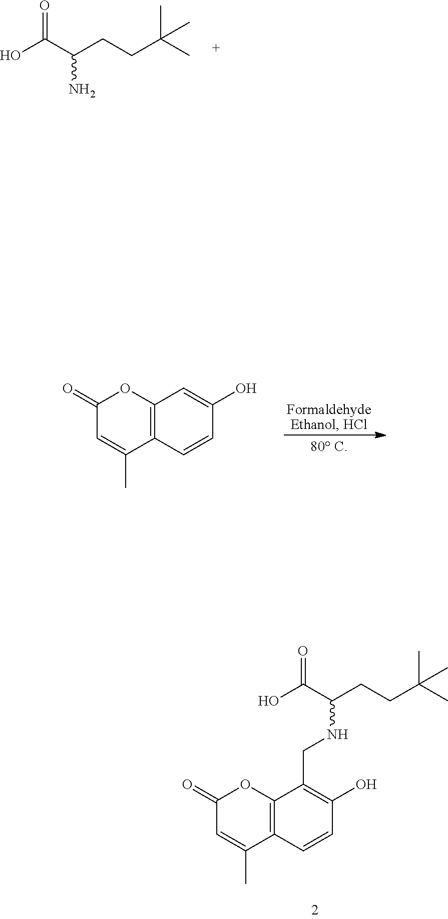 [SMILES: CC(C)(C)CCC(N)C(=O)O ; Cc1cc(=O)oc2cc(O)ccc12 ; "80° C." ; "FormaldehydeEthanol, HCl" ; Cc1cc(=O)oc2c(CNC(CCC(C)(C)C)C(=O)O)c(O)ccc12]

### Synthesis of rac-2-(((7-hydroxy-4-methyl-2-oxo-2H-chromen-8-yl)methyl)amino)-5,5-dimethylhexanoic acid

2-amino-5,5-dimethylhexanoic acid hydrochloride (100 mg, 0.51 mmol) and formaldehyde (0.084 mL, 3.07 mmol) were added to a solution of 7-Hydroxy-4-methylcoumarin (90 mg, 0.51 mmol, 1 equiv.) in ethanol (2 mL). The mixture was stirred at 80° C. for 16 hours. After cooling down to room temperature the white precipitate was collected by filtration the filter was rinsed with ethanol (5 mL) and 2-(((7-hydroxy-4-methyl-2-oxo-2H-chromen-8-yl)methyl)amino)-5,5-dimethylhexanoic acid (20 mg, 0.058 mmol, 11.2% yield) was isolated as white powder. LCMS (Method 1, 0.826 min; M+H=348.1; calcd. 348.1).

¹H-NMR (400 MHz, DMSO) δ 7.57 (d, J=8.7 Hz, 1H), 6.81 (d, J=8.7 Hz, 1H), 6.15 (s, 1H), 4.13-4.01 (m, 2H), 3.22 (t, J=6.1 Hz, 1H), 2.37 (s, 3H), 1.73-1.51 (m, 2H), 1.32-1.19 (m, 2H), 0.84 (s, 9H).

### Example 3

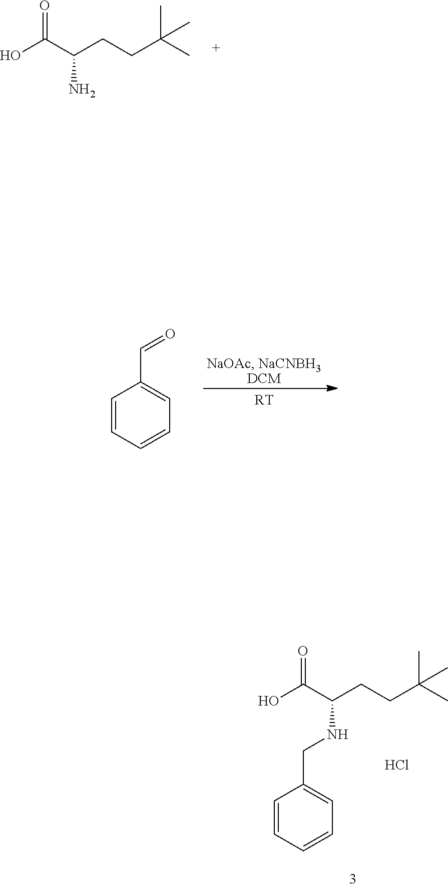

### Synthesis of (S)-2-(benzylamino)-5,5-dimethylhexanoic acid hydrochloride

Benzaldehyde (64 μL, 0.634 mmol, 1.01 equiv.) was added to a mixture of (S)-2-amino-5,5-dimethylhexanoic acid (100 mg, 0.628 mmol) and sodium acetate (77 mg, 0.942 mmol, 1.5 equiv.) in dichloromethane (1 mL). The mixture was stirred at room temperature for 2 hours before addition of sodium cyanoborohydride (79 mg, 1.256 mmol, 2 equiv.). The mixture was stirred at room temperature for 16 hours. The solvent was evaporated in vacuo and the residue was taken up in 1 M HCl in water/methanol (1:1, 2 mL) and purified by basic reversed phase column chromatography (12 g Porapak RXn RP, acetonitrile 5% to 100% in water (0.1M ammonium carbonate). The product containing fractions were combined. During evaporation a white precipitate formed. This was filtered off and the solid was dissolved in 2 mL 2M hydrochloric acid and lyophilized resulting in (S)-2-(benzylamino)-5,5-dimethylhexanoic acid hydrochloride (50 mg, 0.175 mmol, 27% yield, 94.82% purity). LCMS (Method 1, 0.880 min; M+H=250.0; calcd. 250.0).

¹H-NMR (400 MHz, DMSO) δ 14.02 (s, 1H), 9.45 (s, 2H), 7.52 (dq, J=4.8, 2.7 Hz, 2H), 7.48-7.39 (m, 3H), 4.16 (d, J=2.1 Hz, 2H), 3.88 (dd, J=7.1, 4.6 Hz, 1H), 1.97-1.73 (m, 2H), 1.33 (td, J=13.1, 4.9 Hz, 1H), 1.12 (td, J=12.9, 4.4 Hz, 1H), 0.86 (s, 9H).

The following examples were prepared in an analogous manner to Example 3, starting from (S)-2-amino-5,5-dimethyl hexanoic acid (20 mg, 0.126 mmol) and their corresponding aldehyde. Targets were purified by preparative HPLC

| Example | Structure | Yield | LCMS | NMR |
|---|---|---|---|---|
| 4 | 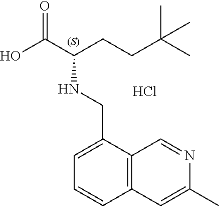 | 50 mg (22% yield, 98.62% purity) | Method 1, 0.907 min; M + H = 315.2; calcd. 315.2 | ¹H-NMR (400 MHz, DMSO) δ 14.16 (brs, 1H), 10.33 (brs, 1H), 9.98 (s, 1H), 9.57 (brs, 1H), 8.42- 8.05 (m, 4H), 4.85 (q, J = 13.5 Hz, 2H), 4.32- 4.22 (m, 1H), 2.85 (s, 3H), |
| | | | | 2.11-1.91 |
| | | | | (m, 2H), 1.40 |
| | | | | (td, J = 12.8, |
| | | | | 5.3 Hz, 1H), |
| | | | | 1.15 (td, J = |
| | | | | 12.6, 4.9 Hz, |
| | | | | 1H), 0.88 (s, |
| | | | | 9H). |
| 5 | 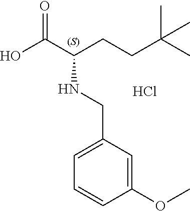 | 85 mg (42% yield, 99.71% purity) | Method 1, 1.058 min; M + H = 280.2; calcd. 280.2 | ¹H-NMR (400 MHz, DMSO) δ 13.95 (s, 1H), 9.70 (d, J = 180.8 Hz, 2H), 7.34 (t, J = 7.9 Hz, 1H), 7.26-7.19 (m, 1H), 7.12- 7.04 (m, 1H), 7.02- 6.94 (m, 1H), 4.19-4.06 |
| | | | | (m, 2H), 3.86- |
| | | | | 3.74 (m, |
| | | | | 4H), 2.01- |
| | | | | 1.77 (m, 2H), |
| | | | | 1.40-1.28 |
| | | | | (m, 1H), 1.18- |
| | | | | 1.06 (m, |
| | | | | 1H), 0.85 (s, |
| | | | | 9H). |
| 25 | 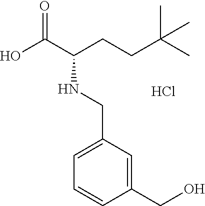 [SMILES: CC(C)(C)CC[C@H](NCc1cccc(CO)c1)C(=O)O ; Cl] | 7.9 mg (4% yield, 99.70% purity) | Method 1, 0.787 min; M + H − HCl = 280.2; calcd. 280.2 Purified by acidic preparative HPLC (Method 4) | ¹H-NMR (400 MHz, DMSO) δ 7.36-7.20 (m, 4H), 5.22 (s, 1H), 4.49 (s, 2H), 3.92- 3.69 (m, 2H), 3.02 (t, J = 6.1 Hz, 1H), 1.61- 1.54 (m, 2H), 1.26- 1.17 (m, 2H), 0.83 (s, 9H) |
| 26 | 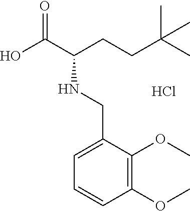 [SMILES: COc1cccc(CN[C@@H](CCC(C)(C)C)C(=O)O)c1OC ; Cl] | 15.7 mg (9% yield, 100% purity) | Method 1, 0.953 min; M + H − HCl = 310.3; calcd. 310.2 | ¹H-NMR (400 MHz, DMSO) δ 13.91 (s, 1H), 9.39- 9.12 (m, 2H), 7.16-7.10 (m, 3H), 4.15 (d, J = 4.4 Hz, 2H), 3.83 (s, 3H), 1.93- 1.78 (m, 2H), 1.34 (td, J = 12.9, 4.8 Hz, |
| | | | | 1H), 1.12 (td, |
| | | | | J = 12.8, 4.6 Hz, |
| | | | | 1H), 0.86 (s, |
| | | | | 9H) |
| 27 | 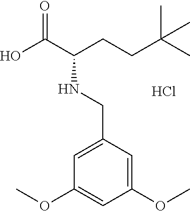 [SMILES: COc1cc(CN[C@@H](CCC(C)(C)C)C(=O)O)cc(OC)c1 ; Cl] | 40.7 mg (24% yield, 100% purity) | Method 1, 0.947 min; M + H − HCl = 310.3; calcd. 310.2 Purified by acidic preparative HPLC (Method | ¹H-NMR (400 MHz, DMSO) δ 9.46 (s, 2H), 6.71 (d, J = 2.3 Hz, 2H), 6.54 (t, J = 2.3 Hz, 1H), 4.08 (s, 2H), 3.80 (t, J = 5.8 Hz, 1H), 3.76 (s, 6H), 1.93- 1.83 (m, 1H), 1.83-1.71 |
| | | | 4) | (m, 1H), 1.32 |
| | | | | (td, J = 13.1, |
| | | | | 5.0 Hz, 1H), |
| | | | | 1.12 (td, J = |
| | | | | 12.8, 4.3 Hz, |
| | | | | 1H), 0.86 (s, |
| | | | | 9H). |
| 28 | 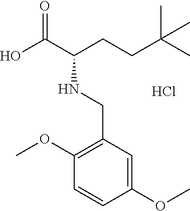 [SMILES: COc1ccc(OC)c(CN[C@@H](CCC(C)(C)C)C(=O)O)c1 ; Cl] | 30.0 mg (18% yield, 100% purity) | Method 1, 0.994 min; M + H − HCl = 310.3; calcd. 310.2 Purified by acidic preparative HPLC (Method 4) | ¹H-NMR (400 MHz, DMSO) δ 13.95 (s, 1H), 9.21 (s, 2H), 7.12 (d, J = 2.8 Hz, 1H), 7.04-6.95 (m, 2H), 4.12 (q, J = 13.2 Hz, 2H), 3.80- 3.75 (m, 4H), 3.73 (s, 3H), 1.93- 1.76 (m, 2H), |
| | | | | 1.32 (td, J = |
| | | | | 12.9, 4.8 Hz, |
| | | | | 1H), 1.12 (td, |
| | | | | J = 12.8, 4.5 Hz, |
| | | | | 1H), 0.86 (s, |
| | | | | 9H). |
| 29 | 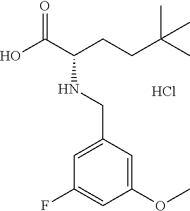 [SMILES: COc1cc(F)cc(CN[C@@H](CCC(C)(C)C)C(=O)O)c1 ; Cl] | 40.8 mg (19% yield, 100% purity) | Method 1, 1.146 min; M + H − HCl = 298.3; calcd. 298.2 Purified by acidic preparative HPLC (Method 4) | ¹H-NMR (400 MHz, DMSO) δ 14.01 (s, 1H), 9.55 (s, 2H), 7.01 (t, J = 1.9 Hz, 1H), 6.97 (dt, J = 9.3, 1.9 Hz, 1H), 6.90 (dt, J = 11.0, 2.3 Hz, 1H), 4.14 (d, J = 1.5 Hz, 2H), 3.86 (dd, J = 7.1, 4.6 Hz, |
| | | | | 1H), 3.80 (s, |
| | | | | 3H), 1.96- |
| | | | | 1.78 (m, 2H), |
| | | | | 1.33 (td, J = |
| | | | | 13.0, 4.8 Hz, |
| | | | | 1H), 1.12 (td, |
| | | | | J = 12.9, 4.4 Hz, |
| | | | | 1H), 0.86 (s, |
| | | | | 9H). |
| 30 | 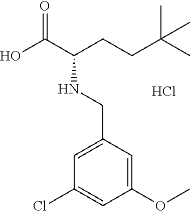 [SMILES: COc1cc(Cl)cc(CN[C@@H](CCC(C)(C)C)C(=O)O)c1 ; Cl] | 23.3 mg (10% yield, 99.93% purity) | Method 1, 1.203 min; M + H − HCl = 314.3; calcd. 314.2 Purified by acidic preparative HPLC (Method 4) | ¹H-NMR (400 MHz, DMSO) δ 14.04 (s, 1H), 9.58 (s, 2H), 7.20 (t, J = 1.7 Hz, 1H), 7.15 (t, J = 1.9 Hz, 1H), 7.09 (t, J = 2.1 Hz, 1H), 4.19- 4.08 (m, 2H), 3.88 (dd, J = 7.2, 4.6 Hz, 1H), 3.81 (s, |
| | | | | 3H), 1.96- |
| | | | | 1.76 (m, 2H), |
| | | | | 1.33 (td, J = |
| | | | | 13.1, 4.9 Hz, |
| | | | | 1H), 1.12 (td, |
| | | | | J = 12.8, 4.3 Hz, |
| | | | | 1H), 0.86 (s, |
| | | | | 9H). |
| 31 | 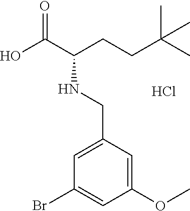 [SMILES: COc1cc(Br)cc(CN[C@@H](CCC(C)(C)C)C(=O)O)c1 ; Cl] | 83.8 mg (37% yield, 99.86% purity) | Method 1, 1.278 min; M + H − HCl = 358.2; calcd. 358.2 Purified by acidic preparative HPLC (Method 4) | ¹H-NMR (400 MHz, DMSO) δ 9.52 (s, 1H), 7.34-7.30 (m, 1H), 7.24- 7.19 (m, 1H), 7.19- 7.13 (m, 1H), 4.12 (s, 2H), 3.91-3.83 (m, 1H), 3.80 (s, 3H), 1.96- 1.78 (m, 2H), 1.32 (td, J = |
| | | | | 13.0, 4.8 Hz, |
| | | | | 1H), 1.12 (td, |
| | | | | J = 12.9, 4.4 Hz, |
| | | | | 1H), 0.86 (s, |
| | | | | 9H). |

### Example 6

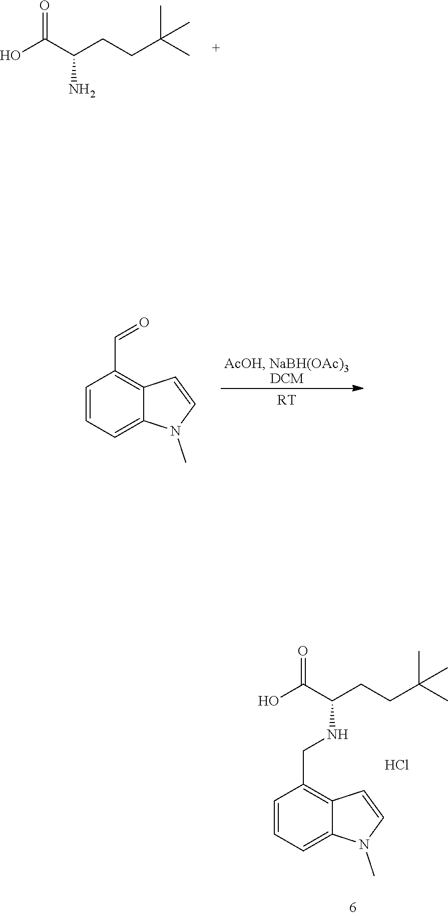

### Synthesis of (S)-5,5-dimethyl-2-(((1-methyl-1H-indol-4-yl)methyl)amino)hexanoic acid hydrochloride

A suspension of 1-methyl-1H-indole-4-carbaldehyde (100 mg, 0.63 mmol, 1 equiv.), (S)-2-amino-5,5-dimethylhexanoic acid (100 mg, 0.63 mmol) and acetic acid (0.036 mL, 0.63 mmol, 1 equiv.) in dichloromethane (1 mL) was stirred at room temperature for 2 hours before addition of sodium triacetoxyborohydride (266 mg, 1.26 mmol, 2 equiv.). The remaining suspension was stirred at room temperature for 16 hours. To the reaction mixture was added aqueous sodium hydroxide (1M, 1 mL) and the layers were separated. The aqueous phase was acidified with aqueous hydrochloric acid (2M). A white precipitate formed and was filtered off. The solid was dissolved in 4M hydrochloric acid in dioxane/water (1:1, 4 mL) and lyophilized resulting in (S)-5,5-dimethyl-2-(((1-methyl-1H-indol-4-yl)methyl)amino)hexanoic acid hydrochloride (66 mg, 0.195 mmol, 31% yield, 99% purity) as off-white solid. LCMS (Method 1, 1.002 min; M+H=303.1; calcd. 303.2).

¹H-NMR (400 MHz, DMSO) δ 7.44-7.32 (m, 2H), 7.18-7.05 (m, 2H), 6.62 (d, J=3.2 Hz, 1H), 4.19-3.99 (m, 2H), 3.79 (s, 3H), 3.10 (t, J=6.1 Hz, 1H), 1.57 (dq, J=11.9, 6.1 Hz, 2H), 1.30-1.12 (m, 2H), 0.81 (s, 9H).

The following Examples were prepared in an analogous manner to Example 6, starting from the corresponding aldehyde.

| Example | Structure | Yield | LCMS | NMR |
|---|---|---|---|---|
| 7 | 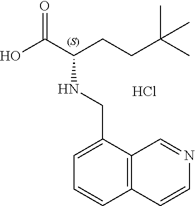 | 37.2 mg, (19% yield, 98.56% purity) | Method 1, 0.863, M + H = 301.1; calcd. 301.1 | ¹H-NMR (400 MHz, DMSO) δ 9.69 (s, 1H), 8.52 (d, J = 5.6 Hz, 1H), 7.89 (d, J = 8.3 Hz, 1H), 7.82 (d, J = 5.6 Hz, 1H), 7.71 (dd, J = 8.2, 7.1 Hz, 1H), 7.60 (d, J = 6.9 Hz, 1H), 4.26 (dd, J = 96.1, 13.1 Hz, 2H), 3.17 (d, J = |
| | | | | 12.4 Hz, 1H), |
| | | | | 1.61-1.47 (m, 2H), |
| | | | | 1.27-1.09 (m, |
| | | | | 2H), 0.79 (s, 9H). |
| 33 | 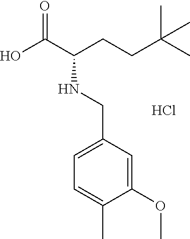 [SMILES: COc1cc(CN[C@@H](CCC(C)(C)C)C(=O)O)ccc1C ; Cl] | 1.2 mg (2% yield, 91.18% purity) | Method 7, 1.084 min; M + H − HCl = 294.3; calcd. 294.2 | n/a |
| 35 | 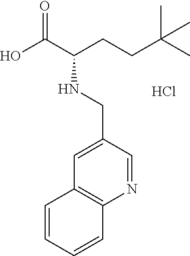 [SMILES: CC(C)(C)CC[C@H](NCc1cnc2ccccc2c1)C(=O)O ; Cl] | 6.0 mg (14% yield, 100% purity) | Method 1, 1.19 min; M + H − HCl = 301.3; calcd. 301.4 | n/a |
| 36 | 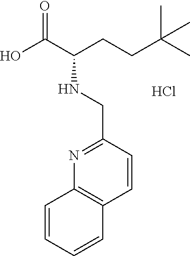 [SMILES: CC(C)(C)CC[C@H](NCc1ccc2ccccc2n1)C(=O)O ; Cl] | 7.2 mg (17% yield, 84.44% purity) | Method 1, 1.26 min; M + H − HCl = 301.3; calcd. 301.4 | n/a |
| 37 | 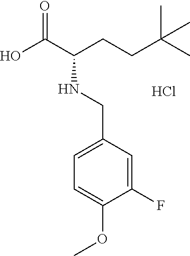 [SMILES: COc1ccc(CN[C@@H](CCC(C)(C)C)C(=O)O)cc1F ; Cl] | 2.1 mg (4% yield, 100% purity) | Method 1, 0.925 min; M + H − HCl − = 298.5; calcd. 298.2 | n/a |
| 41 | 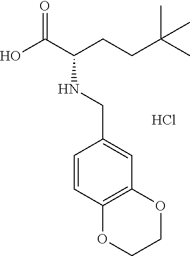 [SMILES: CC(C)(C)CC[C@H](NCc1ccc2c(c1)OCCO2)C(=O)O ; Cl] | 10.8 mg (22% yield, 100% purity) | Method 1, 0.957 min; M + H − HCl = 308.6; calcd. 308.2 | ¹H-NMR (400 MHz, DMSO) δ 13.88 (br, 1H), 9.27 (s, 2H), 7.04 (d, J = 2.0 Hz, 1H), 6.94 (dd, J = 8.2, 2.0 Hz, 1H), 6.90 (d, J = 8.3 Hz, 1H), 4.25 (s, 4H), 4.03 (s, 2H), 3.78 (t, J = 5.8 Hz, 1H), 1.94- 1.68 (m, 2H), 1.31 (td, J = 13.0, 4.7 Hz, 1H), 1.11 (td, J = 12.9, 4.5 |
| | | | | Hz, 1H), 0.85 (s, |
| | | | | 9H). |
| 42 | 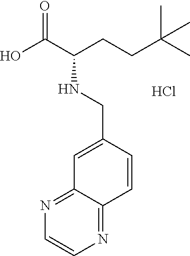 [SMILES: CC(C)(C)CC[C@H](NCc1ccc2nccnc2c1)C(=O)O ; Cl] | 12.9 mg (28% yield, 98.52% purity). | Method 1, 1.034 min; M + H − HCl = 302.2; calcd. 302.2 | n/a |
| 43 | 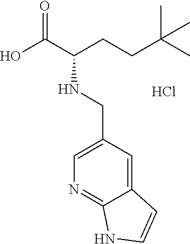 [SMILES: CC(C)(C)CC[C@H](NCc1cnc2[nH]ccc2c1)C(=O)O ; Cl] | 11.4 mg (27% yield, 99.38% purity) | Method 1, 1.028 min; M + H − HCl − = 290.3; calcd. 290.2 | n/a |
| 44 | 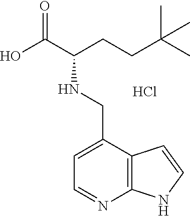 [SMILES: CC(C)(C)CC[C@H](NCc1ccnc2[nH]ccc12)C(=O)O ; Cl] | 2.5 mg (6% yield, 99.19% purity) | Method 1, 0.993 min; M + H − HCl − = 290.3; calcd. 290.2 | n/a |
| 46 | 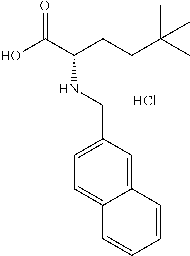 [SMILES: CC(C)(C)CC[C@H](NCc1ccc2ccccc2c1)C(=O)O ; Cl] | 8.5 mg (20% yield, 99.88% purity) | Method 1, 1.148 min; M + H − HCl − = 301.3; calcd. 301.2 | n/a |
| 47 | 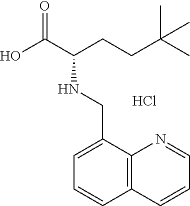 [SMILES: CC(C)(C)CC[C@H](NCc1cccc2cccnc12)C(=O)O ; Cl] | 11.7 mg (28% yield, 98.37% purity) | Method 1, 1.31 min; M + H − HCl = 301.3; calcd. 301.4 | n/a |
| 48 | 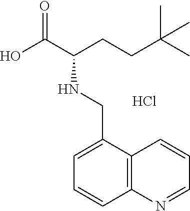 [SMILES: CC(C)(C)CC[C@H](NCc1cccc2ncccc12)C(=O)O ; Cl] | 11.9 mg (28% yield, 98.16% purity) | Method 1, 1.12 min; M + H − HCl = 301.3; calcd. 301.4 | n/a |
| 49 | 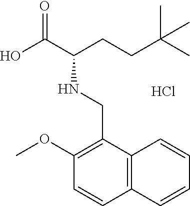 [SMILES: COc1ccc2ccccc2c1CN[C@@H](CCC(C)(C)C)C(=O)O ; Cl] | 2.3 mg (5% yield, 96.31% purity) | Method 3, 1.25 min; M + H − HCl = 330.4; calcd. 330.4 | n/a |
| 50 | 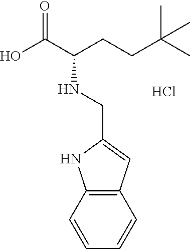 [SMILES: CC(C)(C)CC[C@H](NCc1cc2ccccc2[nH]1)C(=O)O ; Cl] | 0.9 mg (2% yield, 92.6% purity) | Method 3, 1.11 min; M + H − HCl = 287.2; calcd. 287.2 | n/a |
| 51 | 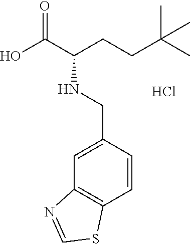 [SMILES: CC(C)(C)CC[C@H](NCc1ccc2scnc2c1)C(=O)O ; Cl] | 11.6 mg (27% yield, 87.79% purity) | Method 3, 0.94 min; M + H − HCl = 307.3; calcd. 307.4 | n/a |
| 52 | 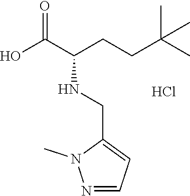 [SMILES: Cn1nccc1CN[C@@H](CCC(C)(C)C)C(=O)O ; Cl] | 16.0 mg (44% yield, 100% purity) | Method 1, 0.87 min; M + H − HCl = 254.2; calcd. 254.3 | n/a |
| 53 | 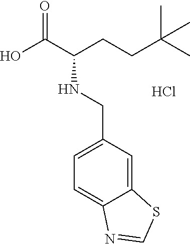 [SMILES: CC(C)(C)CC[C@H](NCc1ccc2ncsc2c1)C(=O)O ; Cl] | 2.3 mg (5% yield, 98.49% purity) | Method 1, 0.918 min; M + H − HCl − = 307.3; calcd. 307.2 | n/a |
| 54 | 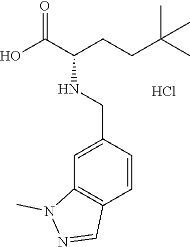 [SMILES: Cn1ncc2ccc(CN[C@@H](CCC(C)(C)C)C(=O)O)cc21 ; Cl] | 4.0 mg (9% yield, 99.08% purity) | Method 1, 1.03 min; M + H − HCl = 304.4; calcd. 304.4 | n/a |
| 55 | 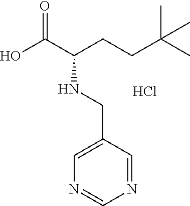 [SMILES: CC(C)(C)CC[C@H](NCc1cncnc1)C(=O)O ; Cl] | 10.1 mg (28% yield, 98.90% purity) | Method 1, 0.830 min; M + H − HCl − = 252.2; calcd. 252.2 | n/a |
| 56 | 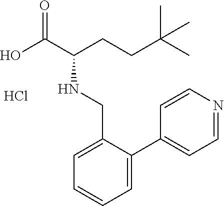 [SMILES: Cl ; CC(C)(C)CC[C@H](NCc1ccccc1-c1ccncc1)C(=O)O] | 5.8 mg (13% yield, 99.89% purity) | Method 1, 0.83 min; M + H − HCl = 327.3; calcd. 327.4 | n/a |
| 57 | 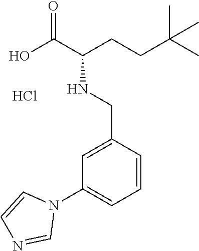 [SMILES: CC(C)(C)CC[C@H](NCc1cccc(-n2ccnc2)c1)C(=O)O ; Cl] | 8.2 mg (18% yield, 100% purity) | Method 1, 0.83 min; M + H − HCl = 316.3; calcd. 316.4 | n/a |
| 58 | 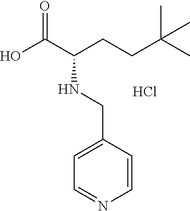 [SMILES: CC(C)(C)CC[C@H](NCc1ccncc1)C(=O)O ; Cl] | 7.5 mg (20% yield, 100% purity) | Method 1, 0.870 min; M + H − HCl − = 251.1; calcd. 251.2 | n/a |
| 61 | 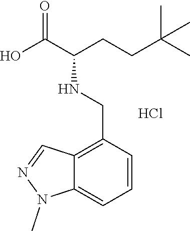 [SMILES: Cn1ncc2c(CN[C@@H](CCC(C)(C)C)C(=O)O)cccc21 ; Cl] | 136 mg (63% yield, 99.87% purity) | Method 1, 0.890 min; M + H − HCl − = 304.6; calcd. 304.2 | ¹H-NMR (400 MHz, DMSO) δ 10.10 (br, 1H), 9.49 (br, 1H), 8.30 (d, J = 1.0 Hz, 1H), 7.79- 7.63 (m, 1H), 7.46 (dd, J = 8.3, 7.0 Hz, 1H), 7.41 (dd, J = 7.1, 1.0 Hz, 1H), 4.56- 4.37 (m, 2H), 4.08 (s, 3H), 3.98- 3.89 (m, 1H), 2.04-1.82 (m, |
| | | | | 2H), 1.36 (td, J = |
| | | | | 13.1, 4.9 Hz, 1H), |
| | | | | 1.12 (td, J = 12.8, |
| | | | | 4.4 Hz, 1H), 0.85 |
| | | | | (s, 9H). |
| 62 | 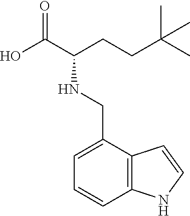 [SMILES: CC(C)(C)CC[C@H](NCc1cccc2[nH]ccc12)C(=O)O] | 44.8 mg (7% yield, 100% purity) | Method 1, 0.927 min; M + H = 289.5; calcd. 289.2 | ¹H-NMR (400 MHz, DMSO) δ 11.24 (s, 1H), 7.42-7.35 (m, 2H), 7.12-7.05 (m, 2H), 6.67- 6.62 (m, 1H), 4.17 (dd, J = 26.0, 13.1 Hz, 2H), 3.24 (t, J = 6.1 Hz, 1H), 1.69- 1.58 (m, 2H), 1.32-1.13 (m, 2H), 0.82 (s, 9H). |
| 67 | 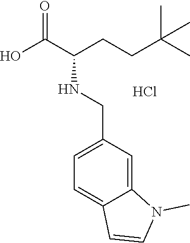 [SMILES: Cn1ccc2ccc(CN[C@@H](CCC(C)(C)C)C(=O)O)cc21 ; Cl] | 2.8 mg (6% yield, 81.9% purity) | Method 1, 1.103 min; M + H − HCl − = 303.4; calcd. 303.2 | n/a |
| 68 | 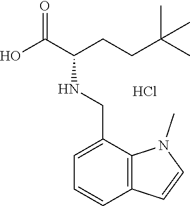 [SMILES: Cn1ccc2cccc(CN[C@@H](CCC(C)(C)C)C(=O)O)c21 ; Cl] | 2.0 mg (4% yield, 99.01% purity) | Method 1, 1.329 min; M + H − HCl − = 303.2; calcd. 303.2 | n/a |
| 69 | 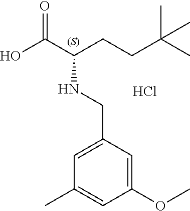 | 13.8 mg (6% yield, 99.90% purity) | Method 1, 1.187 min; M + H − HCl = 294.3; calcd. 294.2 | ¹H-NMR (400 MHz, DMSO) δ 13.99 (brs, 1H), 9.78-9.17 (brs, 2H), 6.96 (s, 1H), 6.88 (s, 1H), 6.82 (s, 1H), 4.08 (s, 2H), 3.89-3.79 (m, 1H), 3.76 (s, 3H), 2.30 (s, 3H), 1.96-1.74 (m, 2H), 1.33 (td, J = 13.1, 4.8 Hz, 1H), 1.12 (td, J = 12.9, |
| | | | | 4.3 Hz, 1H), 0.86 |
| | | | | (s, 9H). |
| 71 | 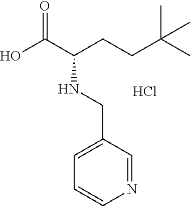 [SMILES: CC(C)(C)CC[C@H](NCc1cccnc1)C(=O)O ; Cl] | 25.0 mg (55% yield, 99.22% purity) | Method 1, 0.745 min; M + H − HCl − = 251.5; calcd. 251.2 | n/a |
| 72 | 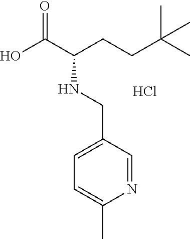 [SMILES: Cc1ccc(CN[C@@H](CCC(C)(C)C)C(=O)O)cn1 ; Cl] | 18.1 mg (38% yield, 97.60% purity) | Method 1, 0.794 min; M + H − HCl − = 265.5; calcd. 265.2 | n/a |
| 74 | 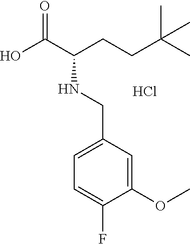 [SMILES: COc1cc(CN[C@@H](CCC(C)(C)C)C(=O)O)ccc1F ; Cl] | 9.1 mg (21% yield, 97.80% purity) | Method 1, 1.257 min; M + H − HCl − = 298.3; calcd. 298.2 | n/a |
| 75 | 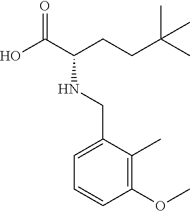 [SMILES: COc1cccc(CN[C@@H](CCC(C)(C)C)C(=O)O)c1C] | 65.5 mg (30% yield, 98.87% purity) | Method 1, 1.027 min; M + H − HCl = 294.5; calcd. 294.2 | ¹H NMR (400 MHz, DMSO) δ 14.41-13.67 (br, 1H), 9.19 (s, 1H), 7.25 (t, J = 7.9 Hz, 1H), 7.10 (d, J = 7.0 Hz, 1H), 7.03 (d, J = 8.0 Hz, 1H), 4.15 (dd, J = 13.2, 5.1 Hz, 2H), 3.98 (t, J = 5.9 Hz, 1H), 3.80 (s, 3H), 2.20 (s, 3H), 1.97-1.77 |
| | | | | (m, 2H), 1.35 (td, |
| | | | | J = 13.1, 4.9 Hz, |
| | | | | 1H), 1.14 (td, J = |
| | | | | 12.8, 4.5 Hz, 1H), |
| | | | | 0.86 (s, 9H). |
| 76 | 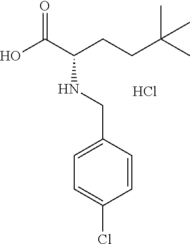 [SMILES: CC(C)(C)CC[C@H](NCc1ccc(Cl)cc1)C(=O)O ; Cl] | 1.2 mg (3% yield, 82.20% purity) | Method 1, 1.263 min; M + H − HCl − = 284.1; calcd. 284.2 | n/a |
| 77 | 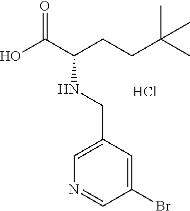 [SMILES: CC(C)(C)CC[C@H](NCc1cncc(Br)c1)C(=O)O ; Cl] | 2.4 mg (5% yield, 100% purity) | Method 1, 1.262 min; M + H − HCl − = 329.2; calcd. 329.1 | n/a |
| 79 | 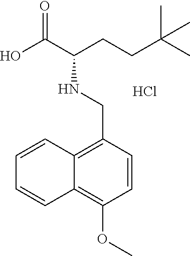 [SMILES: COc1ccc(CN[C@@H](CCC(C)(C)C)C(=O)O)c2ccccc12 ; Cl] | 0.6 mg (1% yield, 100% purity) | Method 1, 1.252 min; M + H − HCl − = 330.5; calcd. 330.2 | n/a |
| 80 | 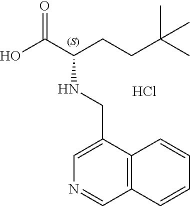 | 2.3 mg, (5% yield, 99.47% purity) | Method 1, 0.886, M + H − HCl = 301.2; calcd. 301.2 | n/a |
| 81 | 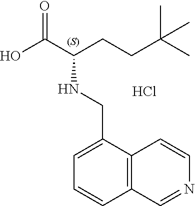 | 5.1 mg, (12% yield, 100% purity) | Method 1, 0.847, M + H − HCl = 301.2; calcd. 301.2 | n/a |
| 83 | 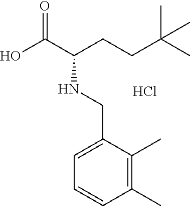 [SMILES: Cc1cccc(CN[C@@H](CCC(C)(C)C)C(=O)O)c1C ; Cl] | 5.5 mg (2% yield, 100% purity) | Method 1, 1.060 min; M + H − HCl = 278.6; calcd. 278.2 | n/a |
| 85 | 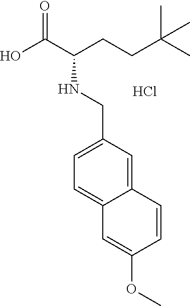 [SMILES: COc1ccc2cc(CN[C@@H](CCC(C)(C)C)C(=O)O)ccc2c1 ; Cl] | 0.9 mg (2% yield, 98.19% purity) | Method 1, 1.290 min; M + H − HCl − = 330.4; calcd. 330.2 | n/a |
| 86 | 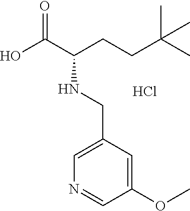 [SMILES: COc1cncc(CN[C@@H](CCC(C)(C)C)C(=O)O)c1 ; Cl] | 16.0 mg (32% yield, 100% purity) | Method 1, 0.828 min; M + H − HCl − = 281.6; calcd. 281.2 | n/a |
| 87 | 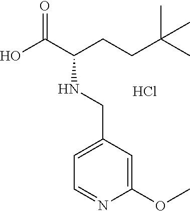 [SMILES: COc1cc(CN[C@@H](CCC(C)(C)C)C(=O)O)ccn1 ; Cl] | 4.6 mg (11% yield, 100% purity) | Method 1, 1.125 min; M + H − HCl − = 281.3; calcd. 281.2 | n/a |
| 88 | 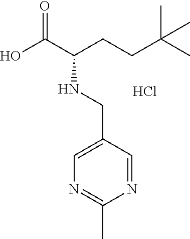 [SMILES: Cc1ncc(CN[C@@H](CCC(C)(C)C)C(=O)O)cn1 ; Cl] | 10.1 mg (26% yield, 99.24% purity) | Method 1, 1.004 min; M + H − HCl − = 266.2; calcd. 266.2 | n/a |
| 89 | 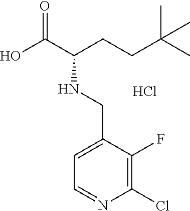 [SMILES: CC(C)(C)CC[C@H](NCc1ccnc(Cl)c1F)C(=O)O ; Cl] | 11.7 mg (27% yield, 100% purity) | Method 1, 1.190 min; M + H − HCl − = 303.2; calcd. 303.1 | n/a |
| 91 | 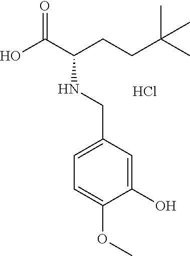 [SMILES: COc1ccc(CN[C@@H](CCC(C)(C)C)C(=O)O)cc1O ; Cl] | 8.3 mg (19% yield, 90.08% purity) | Method 1, 0.923 min; M + H − HCl − = 296.3; calcd. 296.2 | n/a |
| 93 | 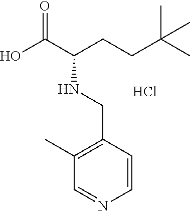 [SMILES: Cc1cnccc1CN[C@@H](CCC(C)(C)C)C(=O)O ; Cl] | 22.9 mg (60% yield, 100% purity) | Method 1, 0.786 min; M + H − HCl − = 265.5; calcd. 265.2 | n/a |
| 95 | 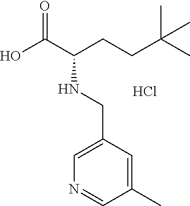 [SMILES: Cc1cncc(CN[C@@H](CCC(C)(C)C)C(=O)O)c1 ; Cl] | 21.8 mg (46% yield, 99.39% purity) | Method 1, 0.811 min; M + H − HCl − = 265.5; calcd. 265.2 | n/a |
| 100 | 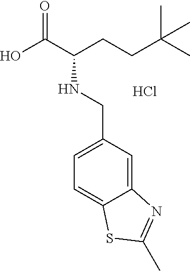 [SMILES: Cc1nc2cc(CN[C@@H](CCC(C)(C)C)C(=O)O)ccc2s1 ; Cl] | 8.8 mg (8% yield, 98.81% purity) | Method 1, 0.917 min; M + H − HCl − = 321.6; calcd. 321.2 | ¹H-NMR (400 MHz, DMSO) δ 9.76-9.36 (m, 2H), 8.11 (d, J = 8.2 Hz, 1H), 8.09 (d, J = 1.6 Hz, 1H), 7.54 (dd, J = 8.3, 1.7 Hz, 1H), 4.34-4.29 (m, 2H), 3.96-3.87 (m, 1H), 2.82 (s, 3H), 1.99-1.76 (m, 2H), 1.34 (td, J = 13.1, 4.9 Hz, 1H), 1.12 (td, J = 12.9, 4.5 Hz, 1H), 0.86 (s, 9H). |
| 103 | 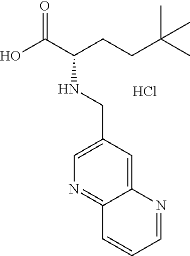 [SMILES: CC(C)(C)CC[C@H](NCc1cnc2cccnc2c1)C(=O)O ; Cl] | 62.4 mg (58% yield, 99.82 % purity) | Method 1, 0.789 min; M + H − HCl = 302.5; calcd. 302.2 | ¹H-NMR (400 MHz, DMSO) δ 14.03 (br, 1H), 10.15-9.57 (m, 2H), 9.13 (d, J = 2.1 Hz, 1H), 9.09 (dd, J = 4.1, 1.7 Hz, 1H), 8.66 (d, J = 2.2 Hz, 1H), 8.51 (dd, J = 8.5, 1.7 Hz, 1H), 7.87 (dd, J = 8.5, 4.2 Hz, 1H), 4.59- 4.41 (m, 3H), 4.13-4.10 (m, 1H), 2.03-1.80 |
| | | | | (m, 2H), 1.36 (td, |
| | | | | J = 13.0, 5.1 Hz, |
| | | | | 1H), 1.14 (td, J = |
| | | | | 12.8, 4.5 Hz, 1H), |
| | | | | 0.87 (s, 9H). |
| 106 | 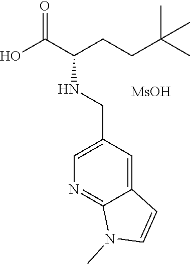 [SMILES: "MsOH" ; Cn1ccc2cc(CN[C@@H](CCC(C)(C)C)C(=O)O)cnc21] | 38.7 mg (29% yield, 99.56% purity) | Method 1, 0.857 min; M + H − MsOH = 304.1; calcd. 304.2 | ¹H-NMR (400 MHz, DMSO) δ 9.37-9.07 (br, 2H), 8.34 (d, J = 2.0 Hz, 1H), 8.08 (d, J = 2.0 Hz, 1H), 7.60 (d, J = 3.4 Hz, 1H), 6.55 (d, J = 3.4 Hz, 1H), 4.31 (s, 2H), 4.03-3.95 (m, 1H), 3.84 (s, 3H), 2.30 (s, 3H), 1.92- 1.76 (m, 2H), 1.32 (td, J = 13.1, 4.9 Hz, 1H), 1.12 |
| | | | | (td, J = 12.9, 4.5 |
| | | | | Hz, 1H), 0.86 (s, |
| | | | | 9H). |
| 110 | 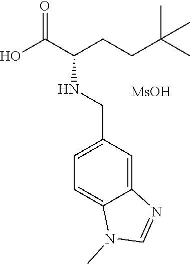 [SMILES: "MsOH" ; Cn1cnc2cc(CN[C@@H](CCC(C)(C)C)C(=O)O)ccc21] | 9.8 mg (7% yield, 99.23% purity) | Method 1, 0.840 min; M + H − MsOH − = 304.5; calcd. 304.2 | ¹H-NMR (400 MHz, DMSO) δ 9.55-8.77 (br, 2H), 8.27 (s, 1H), 7.79 (d, J = 1.5 Hz, 1H), 7.64 (d, J = 8.3 Hz, 1H), 7.37 (dd, J = 8.3, 1.6 Hz, 1H), 4.27 (s, 2H), 3.86 (s, 3H), 3.85-3.79 (m, 1H), 2.29 (s, 3H), 1.90-1.70 (m, 2H), 1.31 (td, J = 13.0, 4.8 Hz, 1H), 1.12 (td, J = 12.8, 4.5 Hz, 1H), 0.85 (s, 9H). |
| 116 | 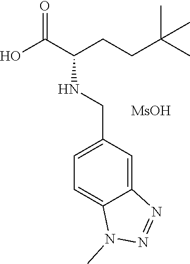 [SMILES: "MsOH" ; Cn1nnc2cc(CN[C@@H](CCC(C)(C)C)C(=O)O)ccc21] | 12.4 mg (9% yield, 98.33% purity) | Method 1, 0.760 min; M + H − MsOH − = 305.4; calcd. 305.2 | ¹H-NMR (400 MHz, DMSO) δ 9.63-8.94 (br, 1H), 8.16 (s, 1H), 7.93 (d, J = 8.6 Hz, 1H), 7.65 (dd, J = 8.6, 1.5 Hz, 1H), 4.40-4.28 (m, 5H), 3.96- 3.85 (m, 1H), 2.29 (s, 3H), 1.94- 1.71 (m, 2H), 1.39-1.23 (m, 1H), 1.13 (td, J = 12.8, 4.5 Hz, 1H), 0.86 (s, 9H). |
| 117 | 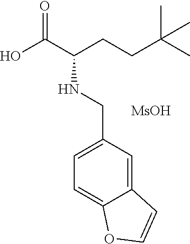 [SMILES: "MsOH" ; CC(C)(C)CC[C@H](NCc1ccc2occc2c1)C(=O)O] | 11.8 mg (7% yield, 99.17% purity) | Method 1, 0.986 min; M + H − MsOH = 290.4; calcd. 290.2 | ¹H NMR (400 MHz, DMSO) δ 9.46-8.83 (br, 2H), 8.07 (d, J = 2.2 Hz, 1H), 7.78 (d, J = 1.8 Hz, 1H), 7.68 (d, J = 8.4 Hz, 1H), 7.42 (dd, J = 8.4, 1.8 Hz, 1H), 7.05 (d, J = 3.0 Hz, 1H), 4.25 (s, 2H), 3.97- 3.80 (m, 1H), 2.29 (s, 3H), 1.90- 1.74 (m, 2H), |
| | | | | 1.32 (td, J = 12.9, |
| | | | | 4.8 Hz, 1H), 1.12 |
| | | | | (td, J = 12.7, 4.5 |
| | | | | Hz, 1H), 0.85 (s, |
| | | | | 9H). |
| 125 | 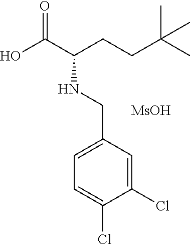 [SMILES: "MsOH" ; CC(C)(C)CC[C@H](NCc1ccc(Cl)c(Cl)c1)C(=O)O] | 18.6 mg (7% yield, 98.30% purity) | Method 1, 1.069 min; M + H − MsOH = 318.4; calcd. 318.1 | ¹H-NMR (400 MHz, DMSO) δ 7.72 (d, J = 2.0 Hz, 1H), 7.67 (d, J = 8.1 Hz, 1H), 7.41 (dd, J = 8.3, 2.0 Hz, 1H), 4.00 (q, J = 13.5 Hz, 2H), 3.60-3.52 (m, 1H), 2.30 (s, 3H), 1.75-1.64 (m, 2H), 1.31- 1.11 (m, 2H), 0.85 (s, 9H). |
| 129 | 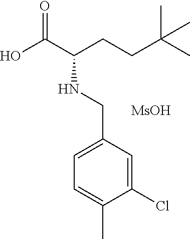 [SMILES: "MsOH" ; Cc1ccc(CN[C@@H](CCC(C)(C)C)C(=O)O)cc1Cl] | 121.4 mg (49% yield, 95.89% purity) | Method 1, 1.066 min; M + H − MsOH − = 298.4; calcd. 298.2 | ¹H-NMR (400 MHz, DMSO) δ 9.45-9.02 (br, 2H), 7.58 (d, J = 1.8 Hz, 1H), 7.43 (d, J = 7.9 Hz, 1H), 7.35 (dd, J = 7.8, 1.8 Hz, 1H), 4.19-4.12 (m, 2H), 2.35 (s, 3H), 2.32 (s, 3H), 1.92- 1.72 (m, 2H), 1.32 (td, J = 13.0, 4.7 Hz, 1H), 1.11 (td, J = 12.9, 4.3 |
| | | | | Hz, 1H), 0.86 (s, |
| | | | | 9H). |
| 132 | 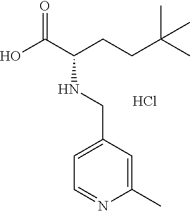 [SMILES: Cc1cc(CN[C@@H](CCC(C)(C)C)C(=O)O)ccn1 ; Cl] | 25.4 mg (53% yield, 97.59% purity) | Method 1, 0.783 min; M + H − HCl − = 265.5; calcd. 265.2 | n/a |

### Example 34

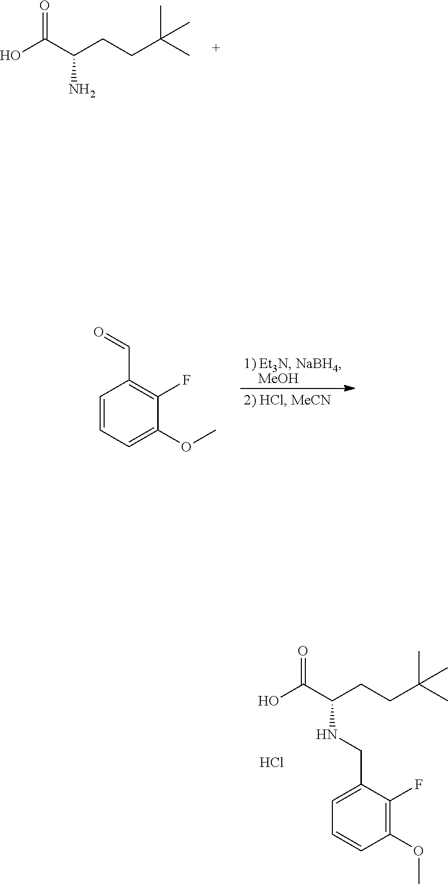

### Synthesis of (S)-2-((2-fluoro-3-methoxybenzyl)amino)-5,5-dimethylhexanoic acid, HCl

A suspension of (S)-2-amino-5,5-dimethylhexanoic acid (75 mg, 1 Eq, 0.47 mmol), 2-fluoro-3-methoxybenzaldehyde (73 mg, 1 eq, 0.47 mmol) and Et₃N (48 mg, 66 μL, 1 Eq, 0.47 mmol) in MeOH (3 mL) was heated at 35° C. for 2 h before being cooled with an ice bath and treated with NaBH₄ (18 mg, 1 Eq, 0.47 mmol) in one portion. The mixture was then allowed to warm to rt before being concentrated to dryness. This was then suspended in water (5 mL) and acetic acid (57 mg, 54 μL, 2 Eq, 0.94 mmol) was added. This was further diluted with water (5 mL) and MeCN (2 mL) before filtering. The filtered solid was then suspended in 1:1 acetone:water (20 mL) at 80° C. for 20 min before cooling to rt and filtering and washing with water (10 mL) and isohexanes (10 mL). The solid was then taken up in water (5 mL) and MeCN (5 mL) before conc. aq. HCl (0.2 mL) was added to afford a solution which was concentrated to afford (S)-2-((2-fluoro-3-methoxybenzyl)amino)-5,5-dimethylhexanoic acid, HCl (122 mg, 0.36 mmol, 76%, 98% Purity) as a colourless solid.

UPLC (Method 3, 0.87 min; M+H=298.3. 1H NMR (500 MHz, DMSO) δ 14.03 (s, 1H), 9.36 (s, 2H), 7.28-7.18 (m, 2H), 7.17-7.12 (m, 1H), 4.25-4.12 (m, 2H), 3.96-3.92 (m, 1H), 3.86 (s, 3H), 1.94-1.74 (m, 2H), 1.33 (app. td, J=13.0, 4.7 Hz, 1H), 1.11 (app. td, J=13.0, 4.4 Hz, 1H), 0.86 (s, 9H). 19F NMR (471 MHz, DMSO) δ −137.89.

The following Examples were prepared in an analogous manner to Example 34, starting from the corresponding aldehyde.

| Example | Structure | Yield | UPLC | NMR |
|---|---|---|---|---|
| 38 | 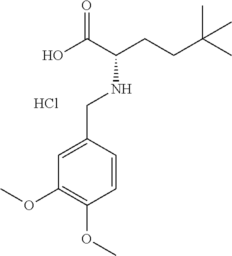 [SMILES: COc1ccc(CN[C@@H](CCC(C)(C)C)C(=O)O)cc1OC ; Cl] | 51 mg, (37% yield, 95% purity) | Method 3, 0.85 min, M + H = 310.4 | 1H NMR (500 MHz, DMSO) δ 13.95 (s, 1H), 9.30 (s, 2H), 7.18 (s, 1H), 7.01-6.97 (m, 2H), 4.09 (s, 2H), 3.80 (s, 1H), 3.78 (s, 3H), 3.77 (s, 3H), 1.93- 1.71 (m, 2H), 1.31 (app. td, J = 13.2, 4.7 Hz, 1H), 1.18-1.07 (m, 1H), 0.86 (s, 9H). |
| 39 | 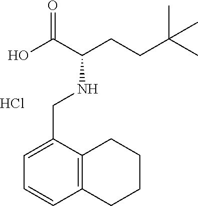 [SMILES: Cl ; CC(C)(C)CC[C@H](NCc1cccc2c1CCCC2)C(=O)O] | 68 mg, (52% yield, 98% purity) | Method 3, 1.16 min, M + H = 304.4 | 1H NMR (500 MHz, DMSO) δ 14.02 (s, 1H), 9.10 (s, 2H), 7.31-7.26 (m, 1H), 7.20-7.10 (m, 2H), 4.16-4.05 (m, 2H), 4.05-3.99 (m, 1H), 2.86- 2.68 (m, 4H), 1.97-1.66 (m, 6H), 1.35 (app. td, J = 13.1, 4.8 Hz, 1H), 1.14 (app. td, J = 13.1, 4.3 Hz, 1H), 0.87 (s, 9H). |
| 40 | 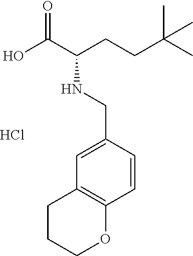 [SMILES: Cl ; CC(C)(C)CC[C@H](NCc1ccc2c(c1)CCCO2)C(=O)O] | 37 mg, (27% yield, 95% purity) | Method 3, 0.98 min, M + H = 306.4 | 1H NMR (500 MHz, DMSO) δ 14.00 (s, 1H), 9.12 (s, 2H), 7.19-7.13 (m, 2H), 6.79-6.74 (m, 1H), 4.17-4.11 (m, 2H), 4.01 (s, 2H), 3.79-3.75 (m, 1H), 2.74 (app. t, J = 6.4 Hz, 2H), 1.92 (app. dt, J = 10.4, 6.2 Hz, 2H), 1.89- 1.71 (m, 2H), 1.36-1.23 (m, 1H), 1.17-1.06 (m, 1H), 0.89-0.79 (m, 9H). |
| 133 | 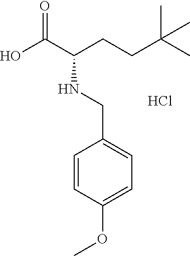 [SMILES: COc1ccc(CN[C@@H](CCC(C)(C)C)C(=O)O)cc1 ; Cl] | 74 mg (49% yield, 98% purity) | Method 3, 0.90 min, M + H = 280.3 | 1H NMR (500 MHz, DMSO) δ 13.98 (s, 1H), 9.28 (s, 2H), 7.45-7.40 (m, 2H), 7.03-6.97 (m, 2H), 4.09 (s, 2H), 3.87- 3.82 (m, 1H), 3.77 (s, 3H), 1.93-1.84 (m, 1H), 1.83- 1.71 (m, 1H), 1.32 (app. td, J = 13.1, 4.6 Hz, 1H), 1.11 (app. td, J = 13.1, 4.4 Hz, 1H), 0.85 (s, 9H). |
| 134 | 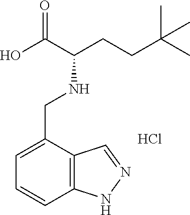 [SMILES: CC(C)(C)CC[C@H](NCc1cccc2[nH]ncc12)C(=O)O ; Cl] | 44 mg, (35% yield, 95% purity) | Method 3, 0.75 min, M + H = 290.3 | 1H NMR (500 MHz, DMSO) δ 14.1 (br.s, 1H), 13.30 (br.s, 1H), 9.64 (s, 1H), 9.41 (s, 1H), 8.30 (s, 1H), 7.61 (d, J = 8.3 Hz, 1H), 7.41 (app.t, J = 7.6 Hz, 1H), 7.31 (d, J= 6.9 Hz, 1H), 4.49 (m, 2H), 4.00 (m, 1H), 1.98-1.78 (m, 2H), 1.35 (app.td, J = 13.1, 4.7 Hz, 1H), 1.12 (app.td, J = 12.9, 4.2 Hz, 1H), 0.85 (s, 9H). |
| 135 | 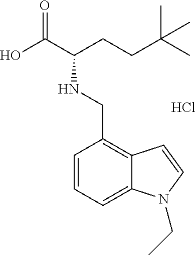 [SMILES: CCn1ccc2c(CN[C@@H](CCC(C)(C)C)C(=O)O)cccc21 ; Cl] | 248 mg, (53% yield, 95% purity) | Method 3, 0.83 min, M + H = 317.4 | 1H NMR (500 MHz, DMSO) δ 13.95 (br.s, 1H), 9.36 (br.s, 2H) δ 7.56 (dd, J = 6.9, 2.4 Hz, 1H), 7.51 (d, J = 3.2 Hz, 1H), 7.28- 7.11 (m, 2H), 6.66 (d, J = 4.1 Hz, 1H), 4.38 (s, 2H), 4.24 (q, J = 7.2 Hz, 2H), 3.94-3.83 (m, 1H), 1.91- 1.74 (m, 2H), 1.37-1.30 (m, 4H), 1.11 (app. td, J = 13.0, 4.6 Hz, 1H), 0.84 (s, 9H). |

### Example 8

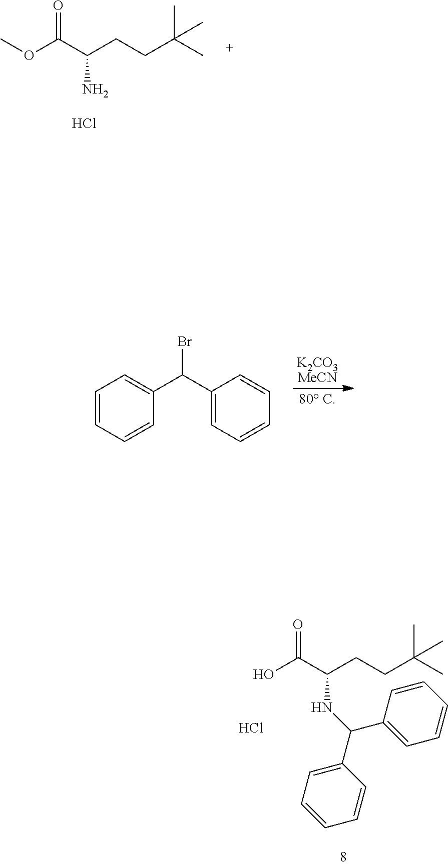

### Synthesis of (S)-2-(benzhydrylamino)-5,5-dimethylhexanoic acid hydrochloride

Bromodiphenylmethane (177 mg, 0.715 mmol) was added to a suspension of potassium carbonate (198 mg, 1.431 mmol) and methyl (S)-2-amino-5,5-dimethylhexanoate hydrochloride (75 mg, 0.358 mmol) in Acetonitrile (1 mL) and stirred at 80° C. for 16 hours. The reaction mixture was filtered and purified by flash chromatography (12 g silica; ethyl acetate 0% to 50% in heptane) to afford methyl (S)-2-(benzhydrylamino)-5,5-dimethylhexanoate (61 mg, 0.126 mmol, 35.2% yield) as white solid. The product was dissolved in acetonitrile (1 mL)/water (1 mL) and lithium hydroxide monohydrate (37.7 mg, 0.898 mmol) was added. The mixture was stirred at 50° C. for 16 hours. The reaction mixture was purified by acidic preparative HPLC (4 g ReproSil-Pur C18, acetonitrile 2-50% in water (+0.1% formic acid), the product containing fractions were combined and lyophillized. The white solid was dissolved in 4 mL MeCN and 1 mL hydrochloric acid (2M) was added and the vail was lyofillized again resulting in (S)-2-(benzhydrylamino)-5,5-dimethylhexanoic acid hydrochloride (46 mg, 0.127 mmol, 35% yield, 92.91% purity) as white solid. LCMS (Method 1, 1.137 min; M+H=326.2; calcd. 326.2).

¹H-NMR (400 MHz, DMSO) δ 10.18 (s, 1H), 7.70 (dd, J=11.6, 7.5 Hz, 4H), 7.48-7.32 (m, 6H), 5.53 (s, 1H), 2.54 (s, 2H), 2.02 (s, 1H), 1.78 (d, J=17.7 Hz, 1H), 1.30-1.19 (m, 2H), 1.06 (t, J=12.1 Hz, 1H), 0.83 (s, 9H). Contains 7% (w/w) DMSO

### Example 9

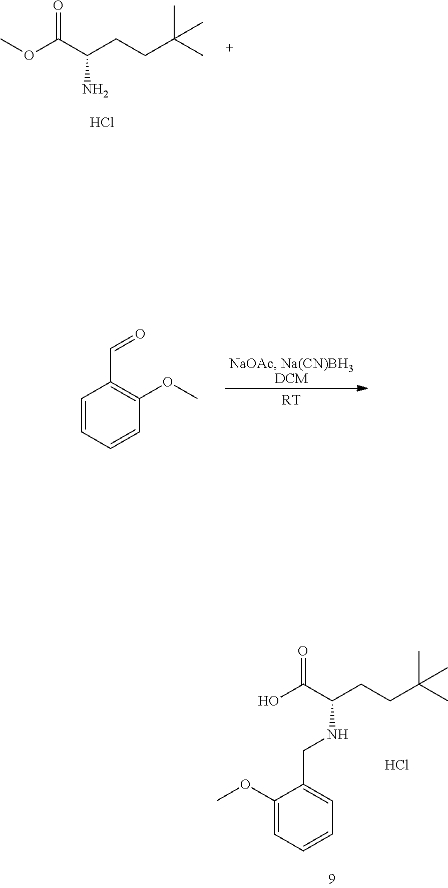

### Synthesis of (S)-5,5-dimethyl-2-(((1-methyl-1H-indol-4-yl)methyl)amino)hexanoic acid hydrochloride

2-methoxybenzaldehyde (64.9 mg, 0.477 mmol) was added to a solution of methyl (S)-2-amino-5,5-dimethylhexanoate hydrochloride (100 mg, 0.477 mmol) and sodium acetate (77 mg, 0.939 mmol) in Dichloromethane (1 mL). The mixture was stirred for 1 h. Sodium cyanoborohydride (80 mg, 1.273 mmol) was added and the mixture was stirred overnight. The solvent was evaporated and the residue was taken up in methanol (1 mL) and water (1 mL). Lithium hydroxide (57.1 mg, 2.384 mmol) was added and the mixture was stirred overnight at 50° C. The solvents were removed and the residue was taken up in DMSO and purified by preparative HPLC, (Method 2). The product containing fractions were combined and lyophillized. The white solid was dissolved in 4M hydrochloric acid in dioxane/water (1:1, 4 mL) and lyophillized resulting in (S)-2-((2-methoxybenzyl)amino)-5,5-dimethylhexanoic acid hydrochloride (66.2 mg, 0.210 mmol, 44% yield, 100% purity). LCMS (Method 1, 0.976 min; M+H=280.2; calcd. 280.2).

¹H-NMR (400 MHz, DMSO) δ 13.98 (s, 1H), 9.25 (s, 2H), 7.47 (dd, J=7.5, 1.7 Hz, 1H), 7.42 (td, J=7.9, 1.7 Hz, 1H), 7.08 (d, J=7.3 Hz, 1H), 7.00 (td, J=7.6, 1.1 Hz, 1H), 4.13 (q, J=13.1 Hz, 2H), 3.82 (s, 3H), 3.77 (dd, J=7.1, 4.7 Hz, 1H), 1.97-1.71 (m, 2H), 1.32 (td, J=13.1, 4.9 Hz, 1H), 1.12 (td, J=12.9, 4.5 Hz, 1H), 0.86 (s, 9H).

The following Examples were prepared in an analogous manner to Example 9, starting from their corresponding aldehyde.

| Example | Structure | Yield | LCMS | NMR |
|---|---|---|---|---|
| 10 | 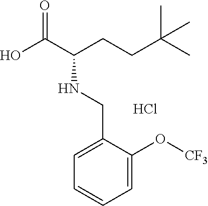 [SMILES: CC(C)(C)CC[C@H](NCc1ccccc1OC(F)(F)F)C(=O)O ; Cl] | 21.3 mg, (12% yield, 99.89% purity) | Method 1, 1.053, M + H = 334.2; calcd. 334.2 | ¹H-NMR (400 MHz, DMSO) δ 13.95 (br, 1H), 9.56 (br, 2H), 7.82 (dd, J = 7.7, 1.7 Hz, 1H), 7.58 (td, J = 7.8, 1.8 Hz, 1H), 7.53-7.41 (m, 2H), 4.23 (dd, J = 13.6, 7.3 Hz, 2H), 3.99-3.91 (m, 1H), 1.98-1.78 (m, 2H), 1.35 (td, J = 12.9, 5.0 Hz, 1H), 1.14 |
| | | | | (td, J = 12.7, 4.7 |
| | | | | Hz, 1H), 0.86 (s, |
| | | | | 9H). |
| 11 | 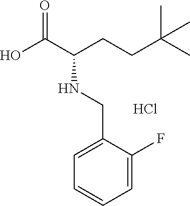 [SMILES: CC(C)(C)CC[C@H](NCc1ccccc1F)C(=O)O ; Cl] | 61.2 mg (42% yield, 99.65% purity) | Method 1, 0.893, M + H = 268.2; calcd. 268.2 | ¹H-NMR (400 MHz, DMSO) δ 13.6 (br, 1H), 9.44 (br 1H), 7.63 (td, J = 7.6, 1.7 Hz, 1H), 7.51-7.46 (m, 1H), 7.31 (dd, J = 4.4, 3.4 Hz, 1H), 7.28 (d, J = 7.9 Hz, 1H), 4.19 (s, 2H), 3.94-3.87 (m, 1H), 1.92-1.78 (m, 2H), 1.33 (td, J = 13.0, 5.2 Hz, 1H), 1.13 |
| | | | | (td, J = 12.7, 4.8 |
| | | | | Hz, 1H), 0.86 (s, |
| | | | | 9H). |
| 12 | 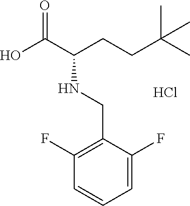 [SMILES: CC(C)(C)CC[C@H](NCc1c(F)cccc1F)C(=O)O ; Cl] | 123.5 mg (80% yield, 93.60% purity) | Method 1, 0.891, M + H = 286.2; calcd. 286.2 | ¹H-NMR (400 MHz, DMSO) δ 14.08 (br, 1H), 9.43 (br, 2H), 7.59 (tt, J = 8.4, 6.6 Hz, 1H), 7.28-7.21 (m, 2H), 4.22 (dd, J = 14.3, 4.4 Hz, 2H), 4.13-4.04 (m, 1H), 1.97-1.78 (m, 2H), 1.35 (td, J = 13.0, 5.1 Hz, 1H), 1.12 (td, J = 12.5, 4.6 Hz, 1H), 0.86 (s, 9H). |
| 13 | 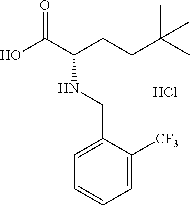 [SMILES: CC(C)(C)CC[C@H](NCc1ccccc1C(F)(F)F)C(=O)O ; Cl] | 104.4 mg (61% yield, 99.72% purity) | Method 1, 1.017, M + H = 318.2; calcd. 318.2 | ¹H-NMR (400 MHz, DMSO) δ 13.78 (br, 1H), 10.07 (br, 2h) 8.04 (d, J = 7.8 Hz, 1H), 7.86-7.74 (m, 2H), 7.64 (t, J = 7.7 Hz, 1H), 4.31 (d, J = 3.5 Hz, 2H), 3.96 (t, J = 6.1 Hz, 1H), 2.03-1.80 (m, 2H), 1.35 (td, J = 13.0, 4.9 Hz, 1H), 1.16 (td, J = 12.8, 4.4 |
| | | | | Hz, 1H), 0.87 (s, |
| | | | | 9H). |

### Example 14

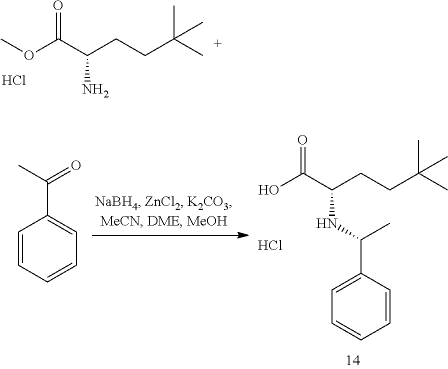

### Synthesis of (S)-5,5-dimethyl-2-(((R)-1-phenylethyl)amino)hexanoic acid hydrochloride

Sodium borohydride (86.9 mg, 2.297 mmol) was added to a suspension of Zinc chloride (157 mg, 1.152 mmol) in 1,2-dimethoxyethane (1.15 mL) at 0° C. The mixture was allowed to warm up to RT and aged for 18h. Acetophenone (69.0 mg, 0.574 mmol) was added to a suspension of methyl (S)-2-amino-5,5-dimethylhexanoate hydrochloride (139 mg, 0.661 mmol) and potassium carbonate (198 mg, 1.433 mmol) in methanol (extra dry) (1.7 mL). The mixture was heated at 50° C. overnight and cooled to RT. The mixture was diluted with acetonitrile (anhydrous) (17 mL). The solution was added to the mixture of Zn(BH₄)₂ at −40° C. After 4 h at −40° C., 1 mL acetone was added and the mixture was allowed to warm up to RT. 5 mL 1M HCl was added slowly. Acetonitrile was removed in vacuo and the mixture was extracted with TBME (3×3 mL). The organic layers were combined and washed with brine, dried over Na₂SO₄ filtered and concentrated. The residue was taken up in DMSO and purified by preparative HPLC, (Method 2). The product containing fractions were combined and lyophillized. The white solid was dissolved in 4M hydrochloric acid in dioxane/water (1:1, 4 mL) and lyophillized resulting in (S)-5,5-dimethyl-2-(((R)-1-phenylethyl)amino)hexanoic acid hydrochloride (12.1 mg, 0.040 mmol, 7% yield, 91.90% purity). LCMS (Method 1, 0.930 min; M+H=264.3; calcd. 264.2).

¹H-NMR (400 MHz, DMSO) δ 9.40 (br, 1H) 7.54-7.38 (m, 5H), 4.36 (d, J=7.1 Hz, 1H), 1.86-1.64 (m, 2H), 1.60 (d, J=6.7 Hz, 3H), 1.22 (td, J=12.9, 4.8 Hz, 1H), 1.06 (td, J=12.8, 4.6 Hz, 1H), 0.81 (s, 9H).

### Example 15

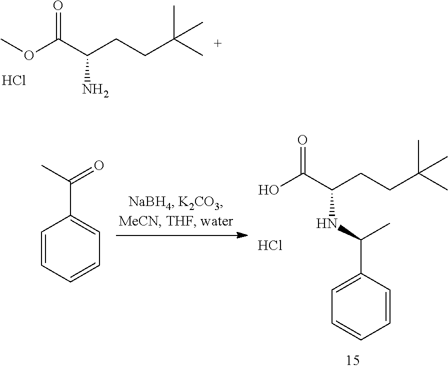

### Synthesis of (S)-5,5-dimethyl-2-(((S)-1-phenylethyl)amino)hexanoic acid hydrochloride

Acetophenone (60.1 mg, 0.5 mmol) was added to a suspension of methyl (S)-2-amino-5,5-dimethylhexanoate hydrochloride (121 mg, 0.575 mmol) and potassium carbonate (225 mg, 1.628 mmol) in methanol (extra dry) (1 mL). The mixture was heated at 50° C. overnight and cooled to RT. The methanol was removed and the residue was suspended in dry tetrahydrofuran (2 mL). The suspension was added to sodium borohydride (151 mg, 3.99 mmol). A 20% (v/v) solution of water/THF (5 mL) was added slowly over 2 hours. The mixture was stirred overnight. The reactions was quenched by the addition of 1 mL of HCl (1M). The mixture was extracted with TBME (3×3 mL). The organic layers were combined and washed with brine, dried over Na₂SO₄ filtered and concentrated. The product was purified by acidic prep (Method 2). The product containing fractions were combined and lyophilised. The white solid was dissolved in 4M hydrochloric acid in dioxane/water (1:1, 4 mL) and lyophilised resulting in (S)-5,5-dimethyl-2-(((S)-1-phenylethyl)amino)hexanoic acid hydrochloride (17.4 mg, 0.058 mmol, 11% yield, 89.33% purity).

LCMS (Method 1, 0.952 min; M+H=264.3; calcd. 264.2).

¹H-NMR (400 MHz, DMSO) δ 13.95 (br, 1H), 9.30 (br, 2H), 7.57-7.50 (m, 2H), 7.48-7.40 (m, 3H), 4.40 (q, J=6.9 Hz, 1H), 3.64-3.54 (m, 1H), 1.95-1.81 (m, 1H), 1.77-1.65 (m, 1H), 1.59 (d, J=6.7 Hz, 3H), 1.27 (td, J=13.1, 4.8 Hz, 1H), 1.03 (td, J=12.9, 4.1 Hz, 1H), 0.85 (s, 9H).

### Example 16

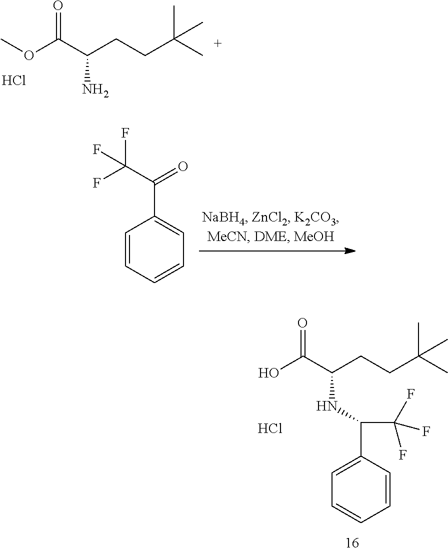 [SMILES: COC(=O)[C@@H](N)CCC(C)(C)C ; Cl ; O=C(c1ccccc1)C(F)(F)F ; Cl ; CC(C)(C)CC[C@H](N[C@@H](c1ccccc1)C(F)(F)F)C(=O)O]

### Synthesis of (S)-5,5-dimethyl-2-(((S)-2,2,2-trifluoro-1-phenylethyl)amino)hexanoic acid

Sodium borohydride (86.9 mg, 2.297 mmol) was added to a suspension of Zinc chloride (157 mg, 1.152 mmol) in 1,2-dimethoxyethane (1.15 mL) at 0° C. The mixture was allowed to warm up to RT and aged for 18 hours. 2,2,2-trifluoro-1-phenylethan-1-one (100 mg, 0.574 mmol) was added to a suspension of methyl (S)-2-amino-5,5-dimethylhexanoate hydrochloride (139 mg, 0.661 mmol) and potassium carbonate (198 mg, 1.433 mmol) in methanol (extra dry) (1.7 mL). The mixture was heated at 50° C. overnight and cooled to room temperature. The mixture was diluted with anhydrous acetonitrile (17 mL). The solution was added to the mixture of Zn(BH₄)₂ at −40° C. After 4 h at −40° C., 1 mL acetone was added and the mixture was allowed to warm up to room temperature. 5 mL 1M HCl was added slowly. Acetonitrile was removed in vacuo and the mixture was extracted with TBME (3×3 mL). The organic layers were combined and washed with brine, dried over Na₂SO₄ filtered and concentrated. The product was purified by acidic prep (Method 2) to afford (S)-5,5-dimethyl-2-(((S)-2,2,2-trifluoro-1-phenylethyl)amino)hexanoic acid (88.4 mg, 0.278 mmol, 48% yield, 99.69% purity). LCMS (Method 1, 1.087 min; M+H=318.3; calcd. 318.2).

¹H-NMR (400 MHz, DMSO) δ 7.47-7.42 (m, 2H), 7.39 (dq, J=7.4, 2.1 Hz, 3H), 4.38 (q, J=8.1 Hz, 1H), 3.20 (d, J=11.9 Hz, 1H), 1.66-1.45 (m, 2H), 1.22 (dd, J=9.8, 7.0 Hz, 2H), 0.85 (s, 9H).

The following Examples were prepared in an analogous manner to Example 16, starting from their corresponding ketone.

| Example | Structure | Yield | LCMS | NMR |
|---|---|---|---|---|
| 17 | 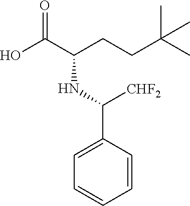 [SMILES: CC(C)(C)CC[C@H](N[C@@H](c1ccccc1)C(F)F)C(=O)O] | 34.9 mg, (20% yield, 98.91% purity) | Method 1, 1.015, M + H = 300.3; calcd. 300.2 | ¹H-NMR (400 MHz, DMSO) δ 7.51 (s, 2H), 7.46-7.37 (m, 3H), 6.44 (t, J = 52.5 Hz, 1H), 4.63-4.29 (m, 1H), 3.46-3.37 (m, 1H), 1.80-1.57 (m, 2H), 1.26 (td, J = 12.8, 4.9 Hz, 1H), 1.15- 1.00 (m, 1H), 0.84 (s, 9H). |
| 21 | 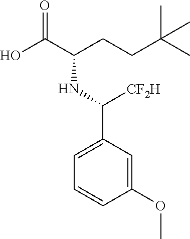 [SMILES: COc1cccc([C@H](N[C@@H](CCC(C)(C)C)C(=O)O)C(F)F)c1] | 95 mg (50% yield; (93.75% purity) | Method 1, 1.076, M + H = 330.3; calcd. 330.2 | ¹H-NMR (400 MHz, DMSO) δ 8.16 (br, 1H), 7.32 (t, J = 7.9 Hz, 1H), 7.05 (s, 1H), 7.00 (d, J = 7.8 Hz, 1H), 6.95 (dd, J = 8.2, 2.5 Hz, 1H), 6.29 (t, J = 54.5 Hz, 1H), 4.17 (s, 1H), 3.76 (s, 3H), 3.36-3.22 (m, 1H), 1.72-1.48 (m, 2H), 1.32-1.18 (m, 1H), 1.11 (dt, J = 17.1, 6.9 Hz, 1H), 0.84 (s, 9H). |
| 24 | 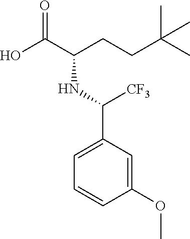 [SMILES: COc1cccc([C@H](N[C@@H](CCC(C)(C)C)C(=O)O)C(F)(F)F)c1] | 25.5 mg (12% yield, 100% purity) | Method 1, 1.307, M + H = 348.3; calcd. 348.2 | ¹H-NMR (400 MHz, DMSO) δ 7.31 (t, J = 7.9 Hz, 1H), 7.03- 6.98 (m, 2H), 6.94 (dd, J = 8.4, 2.5 Hz, 1H), 4.37 (q, J = 8.0 Hz, 1H), 3.76 (s, 3H), 3.19 (t, J = 5.9 Hz, 1H), 1.66-1.45 (m, 2H), 1.36-1.14 (m, 2H), 0.85 (s, 9H). |
| 112 | 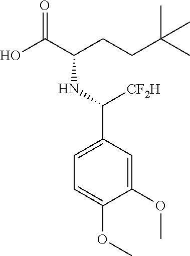 [SMILES: COc1ccc([C@H](N[C@@H](CCC(C)(C)C)C(=O)O)C(F)F)cc1OC] | 162 mg (79% yield, 98.73% purity) | Method 1, 0.986 min; M + H = 360.5; calcd. 360.4 | ¹H-NMR (400 MHz, DMSO) δ 7.00-6.82 (m, 3H), 6.23-5.87 (m, 1H), 3.85-3.69 (m, 8H), 2.99-2.92 (m, 1H), 1.54-1.39 (m, 2H), 1.28-1.09 (m, 2H), 0.84 (s, 9H). |
| 114 | 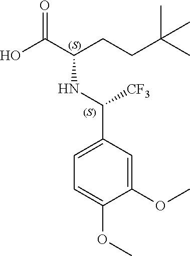 | 65.0 mg (30% yield, 98.20% purity) | Method 1, 1.078 min; M + H = 378.5; calcd. 378.4 | ¹H-NMR (400 MHz, DMSO) δ 7.03 (s, 1H), 6.94 (d, J = 1.0 Hz, 2H), 4.27 (q, J = 8.0 Hz, 1H), 3.75 (s, 3H), 3.74 (s, 3H), 3.12 (t, J = 5.9 Hz, 1H), 1.61-1.46 (m, 2H), 1.27-1.16 (m, 2H), 0.85 (s, 9H). |

### Example 18

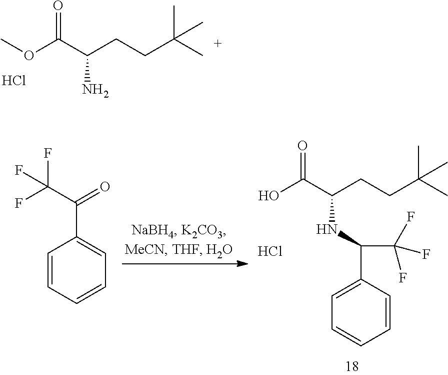 [SMILES: COC(=O)[C@@H](N)CCC(C)(C)C ; Cl ; O=C(c1ccccc1)C(F)(F)F ; Cl ; CC(C)(C)CC[C@H](N[C@H](c1ccccc1)C(F)(F)F)C(=O)O]

### Synthesis of (S)-5,5-dimethyl-2-(((R)-2,2,2-trifluoro-1-phenylethyl)amino)hexanoic acid

2,2,2-trifluoro-1-phenylethan-1-one (87 mg, 0.5 mmol) was added to a suspension of methyl (S)-2-amino-5,5-dimethylhexanoate hydrochloride (121 mg, 0.575 mmol) and potassium carbonate (225 mg, 1.628 mmol) in methanol (extra dry) (1 mL). The mixture was heated at 50° C. overnight and cooled to RT. The methanol was removed and the residue was suspended in tetrahydrofuran (dry) (2 mL). The suspension was added to sodium borohydride (151 mg, 3.99 mmol). A 20% (v/v) solution of water/THF (5 mL) was added slowly over 2 hours. The mixture was stirred overnight. The reactions was quenched by the addition of 1 mL of HCl (1M). The mixture was extracted with TBME (3×3 mL). The organic layers were combined and washed with brine, dried over Na₂SO₄ filtered and concentrated. The product was purified by acidic prep (Method 2) to afford (S)-5,5-dimethyl-2-(((R)-2,2,2-trifluoro-1-phenylethyl)amino)hexanoic acid (55 mg, 0.173 mmol, 30% yield, 99.78% purity). LCMS (Method 1, 1.054 min; M+H=318.4; calcd. 318.2).

¹H-NMR (400 MHz, DMSO) δ 12.52 (br, 1H), 7.53-7.44 (m, 2H), 7.44-7.32 (m, 3H), 4.39 (q, J=7.8 Hz, 1H), 2.86 (t, J=6.4 Hz, 1H), 1.50 (tdd, J=13.3, 8.1, 5.0 Hz, 2H), 1.22 (ddd, J=13.2, 10.6, 6.2 Hz, 1H), 1.05 (ddd, J=13.1, 10.5, 6.7 Hz, 1H), 0.80 (s, 9H).

The following Examples were prepared in an analogous manner to Example 18, starting from their corresponding ketones.

| Example | Structure | Yield | LCMS | NMR |
|---|---|---|---|---|
| 19 | 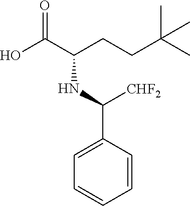 [SMILES: CC(C)(C)CC[C@H](N[C@H](c1ccccc1)C(F)F)C(=O)O] | 40.3 mg, (23% yield, 100% purity) | Method 1, 0.993, M + H = 300.2; calcd. 300.2 | ¹H-NMR (400 MHz, DMSO) δ 7.39-7.31 (m, 5H), 6.07 (td, J = 56.1, 4.7 Hz, 1H), 3.94 (td, J = 11.7, 4.7 Hz, 1H), 2.75 (t, J = 6.3 Hz, 1H), 1.53-1.35 (m, 2H), 1.34-1.17 (m, 1H), 1.15-0.96 (m, 1H), 0.80 (s, 9H). |
| 22 | 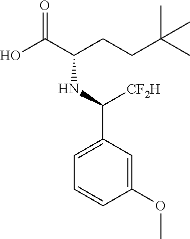 [SMILES: COc1cccc([C@@H](N[C@@H](CCC(C)(C)C)C(=O)O)C(F)F)c1] | 32.6 mg (17% yield, 100% purity) | Method 1, 1.063, M + H = 330.4; calcd. 330.2 | ¹H-NMR (400 MHz, DMSO) δ 7.28 (t, J = 7.8 Hz, 1H), 6.99-6.95 (m, 1H), 6.95-6.89 (m, 2H), 6.07 (dt, J = 55.8, 4.7 Hz, 1H), 3.92 (dt, J = 11.6, 5.7 Hz, 1H), 3.75 (s, 3H), 2.83-2.72 (m, 1H), 1.53- 1.37 (m, 2H), 1.29 (td, J = 12.3, 4.8 Hz, 1H), 1.12- 1.00 (m, 1H), 0.81 (s, 9H). |
| 23 | 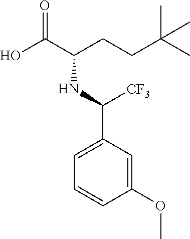 [SMILES: COc1cccc([C@@H](N[C@@H](CCC(C)(C)C)C(=O)O)C(F)(F)F)c1] | 11.1 mg (5% yield, 100% purity) | Method 1, 1.276, M + H = 348.3; calcd. 348.2 | ¹H-NMR (400 MHz, DMSO) δ 7.32 (t, J = 7.9 Hz, 1H), 7.12-6.99 (m, 2H), 6.96 (dd, J = 8.1, 2.7 Hz, 1H), 4.36 (q, J = 7.8 Hz, 1H), 3.76 (s, 3H), 2.85 (t, J = 6.3 Hz, 1H), 1.50 (dq, J = 11.3, 5.5 Hz, 2H), 1.32-1.21 (m, 1H), 1.14-1.03 (m, 1H), 0.80 (s, 9H). |
| 113 | 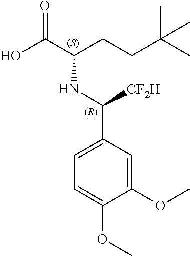 | 60.0 mg (29% yield, 100.00% purity) | Method 1, 0.944 min; M + H = 360.5; calcd. 360.4 | ¹H NMR (400 MHz, DMSO) δ 7.00 (d, J = 1.9 Hz, 1H), 6.93 (d, J = 8.2 Hz, 1H), 6.84 (dd, J = 8.3, 1.9 Hz, 1H), 6.23-5.85 (m, 1H), 3.86 (td, J = 11.1, 5.0 Hz, 1H), 3.75 (brs, 6H), 2.74 (dd, J = 7.7, 5.1 Hz, 1H), 1.53- 1.29 (m, 3H), 1.04 (td, J = 12.5, 4.8 Hz, 1H), 0.81 (s, 9H). |
| 115 | 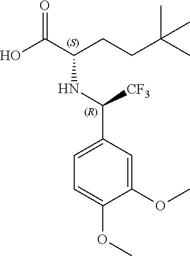 | 70.2 mg (32% yield, 100.00% yield) | Method 1, 1.023 min; M + H = 378.5; calcd. 378.4 | ¹H NMR (400 MHz, DMSO) δ 7.06 (s, 1H), 6.95 (d, J = 1.0 Hz, 2H), 4.30 (q, J = 7.8 Hz, 1H), 3.76 (s, 3H), 3.74 (s, 3H), 2.81 (dd, J = 7.4, 5.4 Hz, 1H), 1.57-1.41 (m, 2H), 1.31 (td, J = 12.4, 4.9 Hz, 1H), 1.11-1.00 (m, 1H), 0.81 (s, 9H). |

### Example 59

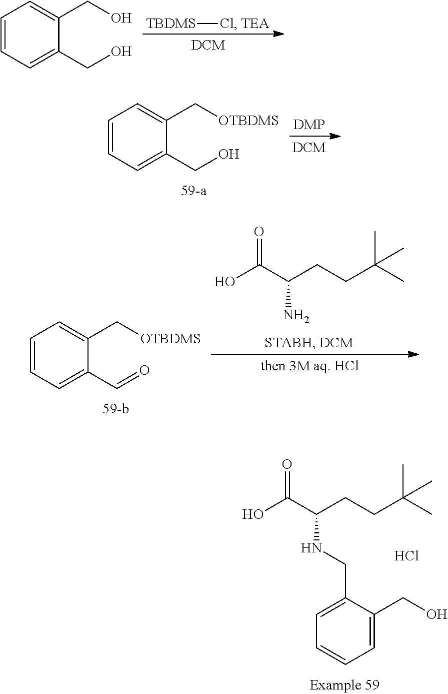

### Preparation of (2-(((tert-butyldimethylsilyl)oxy)methyl)phenyl)methanol (59-a)

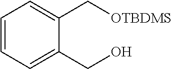 [SMILES: CC(C)(C)[Si](C)(C)OCc1ccccc1CO]

Triethylamine (8.07 mL, 57.9 mmol) was added to a solution of 1,2-Benzenedimethanol (2.0 g, 14.48 mmol) and TBDMS-Cl (1.96 g, 13.03 mmol) in Dichloromethane (5 mL) at 0° C. under nitrogen atmosphere. The mixture was stirred for 2 hours. The reaction mixture was washed with 0.5M aq. hydrochloric acid (10 mL) and extracted with dichloromethane (10 mL). The combined organic layers were dried over Na₂SO₄, filtered and the filtrate was evaporated in vacuo. The crude product was purified by flash chromatography (40 g silica; ethyl acetate 0% to 30% in heptane), affording (2-(((tert-butyldimethylsilyl)oxy)methyl)phenyl)methanol (1.71 g, 6.77 mmol, 46.8% yield) as colorless oil. ¹H-NMR (400 MHz, CDCl₃) δ 7.40-7.28 (m, 4H), 4.81 (s, 2H), 4.68 (d, J=6.5 Hz, 2H), 3.17 (t, J=6.4 Hz, 1H), 0.93-0.90 (m, 9H), 0.13 (t, J=1.0 Hz, 6H).

### Preparation of 2-(((tert-butyldimethylsilyl)oxy)methyl)benzaldehyde (59-b)

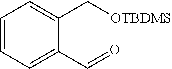 [SMILES: CC(C)(C)[Si](C)(C)OCc1ccccc1C=O]

To (2-(((tert-butyldimethylsilyl)oxy)methyl)phenyl) methanol (1.70 g, 6.73 mmol) in dichloromethane (10 mL) was added Dess-martinperiodinane (3.14 g, 7.41 mmol) and the mixture was stirred at RT for 48 hours. The reaction mixture was washed with sat. aq. sodium bicarbonate (10 mL) and the organic layer was dried over Na₂SO₄, filtered and evaporated, affording 2-(((tert-butyldimethylsilyl)oxy)methyl)benzaldehyde (1.7 g, 6.79 mmol, quant. yield) as white solid. LCMS (Method 6, 1.65 min; M+H=251.2; calcd. 251.4).

### Synthesis of (S)-2-((2-(hydroxymethyl)benzyl)amino)-5,5-dimethylhexanoic acid hydrochloride compound 59

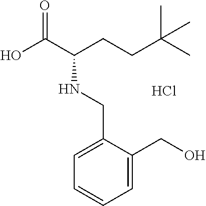 [SMILES: CC(C)(C)CC[C@H](NCc1ccccc1CO)C(=O)O ; Cl]

To 5,5-Dimethyl-L-norleucine (100 mg, 0.628 mmol) in Dichloromethane (2 mL) was added 2-(((tert-butyldimethylsilyl)oxy)methyl)benzaldehyde (236 mg, 0.942 mmol) and the mixture was stirred at RT for 2 hours before addition of sodium triacetoxyborohydride (266 mg, 1.256 mmol). After addition, the mixture was stirred at RT for 16 hours. Reaction mixture was evaporated by heating and the residue was dissolved in 2M hydrochloric acid (2 mL) and stirred for 16 hours at RT. Solids were filtered and the filtrate was purified by acidic prep (Method 2). Product containing fractions were combined, lyophilized, dissolved in 4 mL 1M hydrochloric acid and lyophilized, affording (S)-2-((2-(hydroxymethyl)benzyl)amino)-5,5-dimethylhexanoic acid hydrochloride (66.2 mg, 0.21 mmol, 33.5% yield) as a white solid. LCMS (Method 1, 0.87 min; M+H=280.2; calcd. 280.3). ¹H-NMR (400 MHz, DMSO) δ 9.28 (s, 1H), 7.54-7.52 (m, 1H), 7.48-7.33 (m, 3H), 4.66 (s, 2H), 4.26 (s, 2H), 4.02-4.00 (m, 1H), 1.98-1.77 (m, 2H), 1.34 (td, J=12.9, 4.9 Hz, 1H), 1.13 (td, J=12.8, 4.6 Hz, 1H), 0.86 (s, 9H).

### Example 108

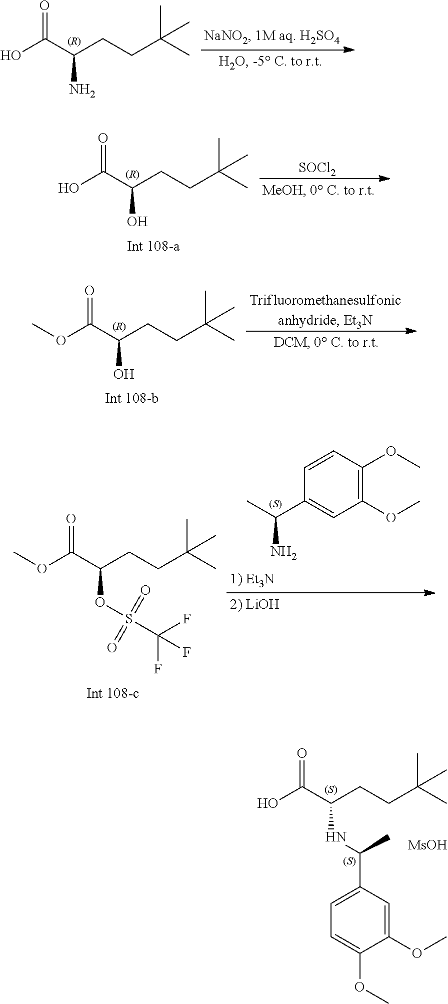

### Synthesis of (R)-2-hydroxy-5,5-dimethylhexanoic acid (int 108-a)

1M aq. sulfuric acid (226 mL, 226 mmol, 3.0 equiv.) was added to (R)-2-amino-5,5-dimethyl hexanoic acid (12 g, 75 mmol, 1.0 equiv.) in water (220 ml). The mixture was cooled to −5° C. and a solution of sodium nitrite (31.2 g, 452 mmol, 6.0 equiv.) in Water (220 ml) was added dropwise, keeping the temperature below 0° C. After addition, the mixture was allowed to warm to room temperature and stirred for 16 hours.

The mixture was extracted with Et₂O (4×200 mL) and the combined organics were washed with brine (300 mL), dried over Na₂SO₄, filtered and concentrated in vacuo to afford (R)-2-hydroxy-5,5-dimethyl hexanoic acid (8.98 g, 56.1 mmol, 74.4% yield) as a yellow solid. 1H-NMR (400 MHz, CDCl₃) δ 4.28 (dd, J=7.2, 4.2 Hz, 1H), 1.92-1.80 (m, 1H), 1.75-1.62 (m, 1H), 1.41-1.27 (m, 2H), 0.90 (s, 9H).

### Synthesis of methyl (R)-2-hydroxy-5,5-dimethylhexanoate (int 108-b)

SOCl₂ (12 ml, 164 mmol, 2.93 equiv.) was added to (R)-2-hydroxy-5,5-dimethyl hexanoic acid (8.98 g, 56.1 mmol, 1 equiv.) in Methanol (120 ml) at 0° C. After addition, the mixture was allowed to warm to room temperature and stirred for 16 hours. The mixture was alkalized to pH 9 by addition of sat. aq. NaHCO₃ and extracted with Et₂O (2×400 mL). Combined organics were dried over Na₂SO₄, filtered and concentrated in vacuo to afford methyl (R)-2-hydroxy-5,5-dimethylhexanoate (10.01 g, 55.0 mmol, 98% yield) as yellow oil. Contains 4.2% (w/w) MeOH. 1H-NMR (400 MHz, CDCl₃) δ 4.18 (dd, J=7.2, 4.2 Hz, 1H), 3.80 (s, 3H), 1.84-1.72 (m, 1H), 1.67-1.52 (m, 1H), 1.37-1.21 (m, 2H), 0.89 (s, 9H).

### Synthesis of methyl (R)-5,5-dimethyl-2-(((trifluoromethyl)sulfonyl)oxy)hexanoate (int 108-c)

Trifluoromethanesulfonic anhydride (4.65 mL, 27.5 mmol, 1.10 equiv.) was added dropwise to a solution of methyl (R)-2-hydroxy-5,5-dimethylhexanoate (4.36 g, 25.02 mmol, 1.0 equiv.) and triethylamine (4.19 ml, 30.0 mmol, 1.2 equiv.) in Dichloromethane (100 mL) at 0° C. After addition, the mixture was allowed to warm to room temperature and stirred for 16 hours. Water (100 mL) was added, and the mixture was extracted with EtOAc (2×250 mL). The combined organics were washed with brine (250 mL), dried over Na₂SO₄, filtered and concentrated in vacuo to afford methyl (R)-5,5-dimethyl-2-(((trifluoromethyl)sulfonyl)oxy)hexanoate (7.14 g, 23.31 mmol, 49% corrected yield) as a dark brown oil. 1H-NMR (400 MHz, CDCl3) δ 5.12 (dd, J=6.9, 5.0 Hz, 1H), 3.85 (s, 3H), 2.05-1.90 (m, 2H), 1.36-1.24 (m, 2H), 0.90 (s, 9H).

### Synthesis of (S)-2-(((S)-1-(3,4-dimethoxyphenyl)ethyl)amino)-5,5-dimethylhexanoic acid compound 108—methanesulfonic acid

Methyl (R)-5,5-dimethyl-2-(((trifluoromethyl)sulfonyl)oxy)hexanoate (70 mg, 0.229 mmol) in Dichloromethane was added dropwise to a solution of (S)-1-(3,4-dimethoxyphenyl)ethan-1-amine (41.4 mg, 0.229 mmol) in dichloromethane with triethylamine (104 μl, 0.743 mmol). The mixture was stirred overnight. The DCM was removed by gentle air flow and the residue was taken up in Acetonitrile (1.000 ml) and Water (1.000 ml). lithium hydroxide (21.89 mg, 0.914 mmol) was added and the mixture was stirred overnight. The mixture was submitted for acidic preparative HPLC (method 2) to afford (S)-2-(((S)-1-(3,4-dimethoxyphenyl)ethyl)amino)-5,5-dimethylhexanoic acid (33.5 mg, 0.104 mmol, 45.3% yield). The product was dissolved in Acetonitrile (1.1 ml) and methanesulfonic acid (0.1M in MeCN) (1040 μl, 0.104 mmol) was added. The mixture was lyophilized to afford (S)-2-(((S)-1-(3,4-dimethoxyphenyl)ethyl)amino)-5,5-dimethylhexanoic acid compound with methanesulfonic acid (43.6 mg, 45.5% yield, 99.67% purity).

LCMS (Method 1, 0.884 mi; M−H− MsOH=322.2; calcd. 322.2). ¹H-NMR (400 MHz, DMSO) δ 14.32-13.95 (br, 1H), 9.47-8.85 (br, 2H), 7.13 (d, J=1.9 Hz, 1H), 7.00 (d, J=8.3 Hz, 1H), 6.95 (dd, J=8.3, 1.9 Hz, 1H), 4.37-4.29 (m, 1H), 3.77 (s, 3H), 3.77 (s, 3H), 3.38-3.29 (m, 1H), 2.31 (s, 3H), 1.79-1.62 (m, 2H), 1.60 (d, J=6.8 Hz, 3H), 1.21 (td, J=12.9, 5.3 Hz, 1H), 1.07 (td, J=12.6, 4.6 Hz, 1H), 0.81 (s, 9H).

The following examples, were prepared in an analogous manner to example 108, starting from the corresponding ester.

| Example | Structure | Yield | LCMS | NMR |
|---|---|---|---|---|
| 63 | 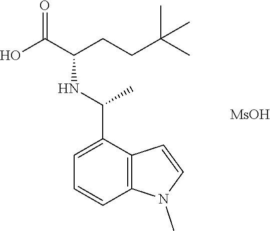 [SMILES: "MsOH" ; C[C@@H](N[C@@H](CCC(C)(C)C)C(=O)O)c1cccc2c1ccn2C] | 20.9 mg (15% yield, 99.40% purity) | Method 7, 1.064 min; M + H − MsOH⁻ = 316.1; calcd. 316.2 | ¹H-NMR (400 MHz, DMSO) δ 7.50 (d, J = 8.0 Hz, 1H), 7.43 (d, J = 3.1 Hz, 1H), 7.34- 7.21 (m, 2H), 6.69 (d, J = 3.2 Hz, 1H), 4.84 (d, J = 7.1 Hz, 1H), 3.81 (s, 3H), 3.51 (s, 1H), 2.30 (s, 3H), 1.86- 1.78 (m, 1H), 1.67 (d, J = 6.6 Hz, 3H), 1.27 (td, J = 13.2, 4.7 Hz, 1H), 0.93 (td, J = 12.9, 4.0 Hz, 1H), 0.83 (s, 9H). |
| 64 | 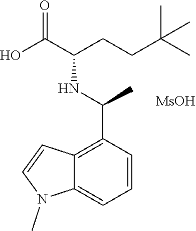 [SMILES: "MsOH" ; C[C@H](N[C@@H](CCC(C)(C)C)C(=O)O)c1cccc2c1ccn2C] | 18.5 mg (16% yield, 100% purity) | Method 7, 1.046 min; M + H − MsOH = 339.2; calcd. 339.2 | ¹H-NMR (400 MHz, DMSO) δ 7.47 (dd, J = 6.4, 2.7 Hz, 1H), 7.41 (d, J = 3.2 Hz, 1H), 7.27-7.20 (m, 2H), 6.51 (d, J = 3.1 Hz, 1H), 4.78-4.66 (m, 1H), 3.81 (s, 3H), 3.18 (s, 1H), 2.29 (s, 3H), 1.69-1.55 (m, 5H), 1.20 (td, J = 12.7, 5.3 Hz, 1H), 1.04 (dt, J = 13.2, 6.5 Hz, 1H), 0.79 (s, 9H). |
| 65 | 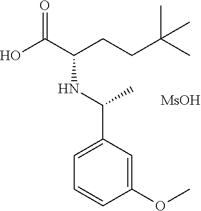 [SMILES: "MsOH" ; COc1cccc([C@@H](C)N[C@@H](CCC(C)(C)C)C(=O)O)c1] | 3.0 g (47% yield, 90.89% purity) | Method 7, 0.988 min; M + H = 294.1; calcd. 294.4 | ¹H-NMR (400 MHz, DMSO) δ 14.35- 13.50 (brs, 1H), 9.24 (brs, 2H), 7.37 (t, J = 8.0 Hz, 1H), 7.15- 7.04 (m, 2H), 6.99 (dd, J = 8.2, 2.6 Hz, 1H), 4.43 (s, 1H), 3.78 (s, 3H), 3.58 (s, 1H), 2.30 (s, 3H), 1.93-1.80 (m, 1H), 1.76-1.64 (m, 1H), 1.58 (d, J = 6.7 Hz, 3H), 1.27 (td, J = |
| | | | | 13.2, 4.8 Hz, 1H), 1.01 |
| | | | | (td, J = 13.0, 4.1 Hz, |
| | | | | 1H), 0.86 (s, 9H). |
| 66 | 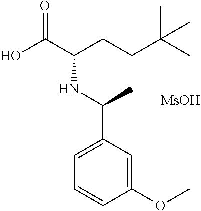 [SMILES: "MsOH" ; COc1cccc([C@H](C)N[C@@H](CCC(C)(C)C)C(=O)O)c1] | 86.1 mg (44% yield, 99.64% purity) | Method 7, 0.947 min; M + H = 294.1; calcd. 294.4 | ¹H-NMR (400 MHz, DMSO) δ 14.06 (brs, 1H), 9.70-9.00 (m, 2H), 7.38 (t, J = 7.9 Hz, 1H), 7.09 (t, J = 2.1 Hz, 1H), 7.01 (td, J = 8.1, 2.0 Hz, 2H), 4.38 (s, 1H), 3.77 (s, 3H), 3.35 (s, 1H), 2.32 (s, 3H), 1.80-1.55 (m, 5H), 1.23 (td, J = 13.0, 5.0 Hz, 1H), 1.07 (td, J = 12.8, 4.5 Hz, 1H), 0.82 (s, 9H). |
| 101 | 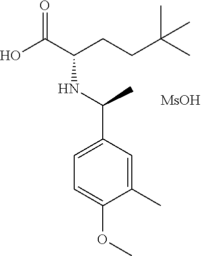 [SMILES: "MsOH" ; COc1ccc([C@H](C)N[C@@H](CCC(C)(C)C)C(=O)O)cc1C] | 40.3 mg (43% yield, 88.00% purity) | Method 1, 1.058 min; M − H − MsOH = 306.4; calcd. 306.2 | ¹H-NMR (400 MHz, DMSO) δ 7.19-7.12 (m, 2H), 6.92 (d, J = 8.2 Hz, 1H), 4.01- 3.90 (m, 1H), 3.78 (s, 3H), 2.92-2.84 (m, 1H), 2.13 (s, 3H), 1.54- 1.46 (m, 2H), 1.40 (d, J = 6.7 Hz, 3H), 1.17- 1.08 (m, 2H), 0.80 (s, 9H). |
| 102 | 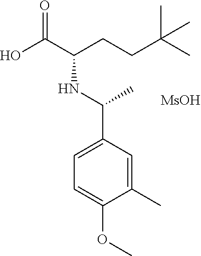 [SMILES: "MsOH" ; COc1ccc([C@@H](C)N[C@@H](CCC(C)(C)C)C(=O)O)cc1C] | 36.4 mg (39% yield, 98.56% purity) | Method 7, 1.087 min; M − H − MsOH = 306.3; calcd. 306.2 | ¹H-NMR (400 MHz, DMSO) δ 14.08- 13.72 (br, 1H), 9.12- 9.03 (br, 2H), 7.33- 7.27 (m, 2H), 6.99 (d, J = 8.3 Hz, 1H), 4.40- 4.30 (m, 1H), 3.80 (s, 3H), 2.31 (s, 3H), 2.15 (s, 3H), 1.90-1.79 (m, 1H), 1.75-1.63 (m, 1H), 1.57 (d, J = 6.8 Hz, 3H), 1.26 (td, J = 13.1, 4.6 Hz, 1H), 1.01 (td, J = 12.9, 3.9 Hz, 1H), 0.86 (s, 9H). |
| 107 | 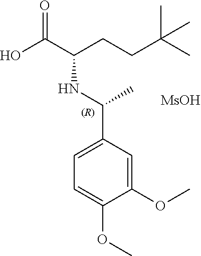 | 47.3 mg (49% yield, 99.02% purity) | Method 7, 0.927 min; M − H − MsOH = 322.2; calcd. 322.2 | ¹H-NMR (400 MHz, DMSO) δ 9.19-9.08 (br, 2H), 7.16 (d, J = 1.9 Hz, 1H), 7.03 (dd, J = 8.3, 1.9 Hz, 1H), 6.99 (d, J = 8.3 Hz, 1H), 4.44-4.36 (m, 1H), 3.77 (s, 3H), 3.77 (s, 3H), 3.52 (s, 1H), 2.30 (s, 3H), 1.92-1.80 (m, 1H), 1.74-1.62 (m, 1H), 1.58 (d, J = 6.7 Hz, 3H), 1.26 (td, J = 13.2, 4.8 Hz, 1H), 1.01 (td, J = 13.0, 4.0 Hz, 1H), 0.86 (s, 9H). |

### Example 131

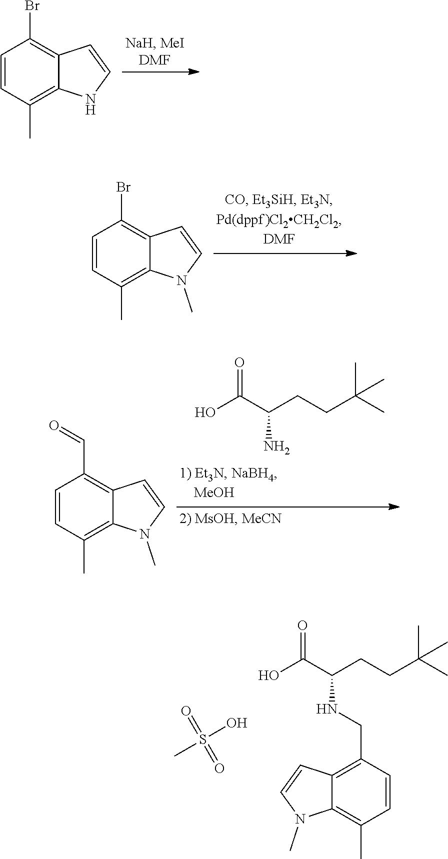

### Synthesis of 4-bromo-1,7-dimethyl-1H-indole intermediate 131-a

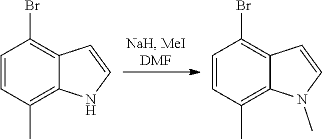

A solution of 4-bromo-7-methyl-1H-indole (606 mg, 1 Eq, 2.88 mmol) in DMF (3.0 mL) was cooled to O ° C. before NaH (60% wt on mineral oil) (0.12 g, 60% Wt, 1.0 Eq, 2.88 mmol) was added. The mixture was allowed to warm to rt and stirred for 15 min before Mel (409 mg, 180 μL, 1.0 Eq, 2.88 mmol) was added. The reaction mixture was stirred at rt for 2 hours before being partitioned between EtOAc (40 mL) and sat. aq. NH₄Cl (30 mL). The layers were separated and the organic phase was washed with 1:1 brine:water (2×30 mL) and brine (30 mL). The organic phase was dried over MgSO4, filtered and concentrated in vacuo to afford 4-bromo-1,7-dimethyl-1H-indole (638 mg, 2.8 mmol, 97%, 98% Purity) as a brown oil which solidified on standing.

LCMS (Method #acid3minb, 2.14 min; M+H=n/a. 1H NMR (500 MHz, DMSO) δ 7.34 (d, J=3.1 Hz, 1H), 7.08 (d, J=7.6 Hz, 1H), 6.78 (dd, J=7.6, 1.0 Hz, 1H), 6.32 (d, J=3.1 Hz, 1H), 4.06 (s, 3H), 2.70 (d, J=0.9 Hz, 3H).

### Preparation of 1,7-dimethyl-1H-indole-4-carbaldehyde 131-b

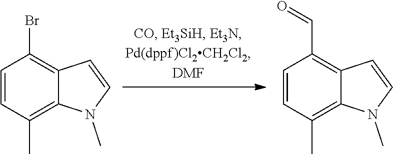

A solution of 4-bromo-1,7-dimethyl-1H-indole (400 mg, 1 Eq, 1.78 mmol), triethylamine (545 mg, 750 μL, 3.01 Eq, 5.38 mmol), triethylsilane (619 mg, 850 μL, 2.98 Eq, 5.32 mmol) and PdCl₂(dppf)-DCM (60 mg, 0.041 Eq, 73 μmol) in DMF (6.0 mL) was degassed for 5 min under a stream of N₂ before being sealed. This was purged with N₂ (×3) before charging with CO (1.5 bar) then the reaction mixture was heated to 90° C. for 6 h. After cooling to rt, the reaction mixture was taken up in EtOAc (40 mL) then washed with sat. aq. NH₄Cl (20 mL), water:brine (2×1:1, 20 mL) and brine (20 mL). The organic phase was dried over MgSO4, filtered and concentrated on to silica (˜1 g). The crude product was purified by chromatography on silica gel (12 g cartridge, 0-70% EtOAc/iHex) to afford 1,7-dimethyl-1H-indole-4-carbaldehyde (195 mg, 1.1 mmol, 62%, 98% Purity) as a yellow solid.

LCMS (Method #acid3minb, 1.68 min; M+H=174.2. 1H NMR (500 MHz, DMSO) δ 10.10 (d, J=0.7 Hz, 1H), 7.53 (d, J=7.4 Hz, 1H), 7.47 (d, J=3.0 Hz, 1H), 7.09-7.04 (m, 2H), 4.11 (s, 3H), 2.83 (s, 3H).

### Preparation of (S)-2-(((1,7-dimethyl-1H-indol-4-yl)methyl)amino)-5,5-dimethylhexanoic acid, Mesylic acid 131

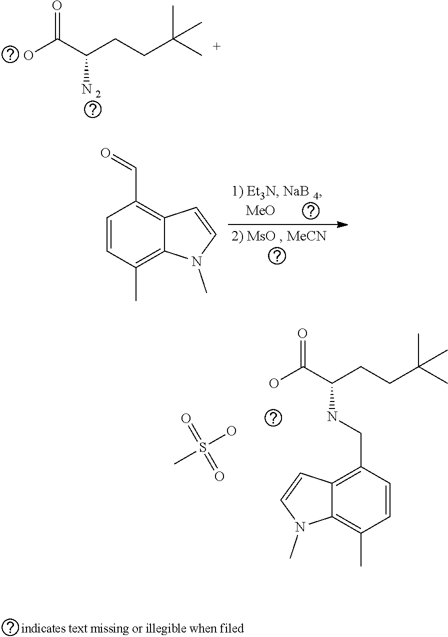

A suspension of (S)-2-amino-5,5-dimethylhexanoic acid (92 mg, 1.0 Eq, 0.58 mmol), 1,7-dimethyl-1H-indole-4-carbaldehyde (100 mg, 1 Eq, 577 μmol) and triethylamine (59 mg, 81 μL, 1.0 Eq, 0.58 mmol) in MeOH (5.0 mL) was stirred at 40° C. for 2 h leading to the formation of a solution. After cooling to 0° C., sodium borohydride (22 mg, 1.0 Eq, 0.58 mmol) was added and the mixture was allowed to warm to rt over 1 h. The mixture was concentrated to dryness then suspended in water (5 mL). Treatment with acetic acid (0.1 mL) ensued before filtering. The material was then suspended in water (10 mL) and acetone (2 mL) before heating at 60° C. for 30 min. After cooling to rt, the mixture was filtered to afford the freebase. The freebase was suspended in MeCN (2 mL) and treated with 0.1 M MsOH in MeCN (1 equiv) and sonicated to briefly afford a solution. This was then concentrated to dryness to afford (S)-2-(((1,7-dimethyl-1H-indol-4-yl)methyl)amino)-5,5-dimethylhexanoic acid, Mesylic acid (52 mg, 0.12 mmol, 21%, 96% Purity) as a colourless solid.

LCMS (Method #acid3minb, 1.49 min; M+Na=339.2. 1H NMR (500 MHz, DMSO) b 9.12 (v. br. s, 2H), 7.32 (d, J=3.2 Hz, 1H), 7.03 (d, J=7.3 Hz, 1H), 6.89 (d, J=7.3 Hz, 1H), 6.57 (d, J=3.2 Hz, 1H), 4.36-4.26 (m, 2H), 4.07 (s, 3H), 3.82-3.76 (m, 1H), 2.74 (s, 3H), 2.30 (s, 3H), 1.88-1.70 (m, 2H), 1.32 (app. td, J=13.1, 4.7 Hz, 1H), 1.10 (app. td, J=12.8, 4.4 Hz, 1H), 0.84 (s, 9H).

### Example 136

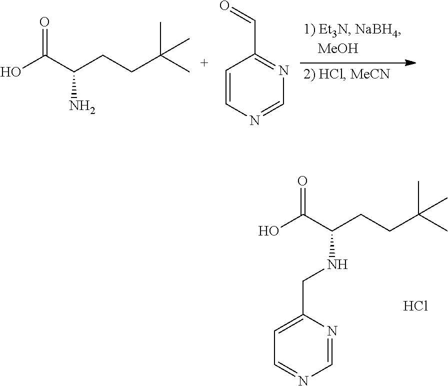

### Synthesis of (S)-5,5-dimethyl-2-((pyrimidin-4-ylmethyl)amino)hexanoic acid, HCl

A suspension of (S)-2-amino-5,5-dimethylhexanoic acid (60 mg, 1 Eq, 0.38 mmol), pyrimidine-4-carbaldehyde (41 mg, 1 Eq, 0.38 mmol) and Et₃N (38 mg, 53 μL, 1 Eq, 0.38 mmol) in MeOH (2 mL) was heated intermittently with a heat gun to afford a solution which was allowed to stir at rt for 2 h before being cooled with an ice bath and treated with NaBH₄ (16 mg, 1.1 Eq, 0.41 mmol) in one portion. The mixture was then allowed to warm to rt before stirring for 1.5 h. The reaction mixture was concentrated to dryness. The residue was suspended in water (5 mL), then acetic acid (45 mg, 43 μL, 2 Eq, 0.75 mmol) was added. This was further diluted with water (5 mL) before solvent was removed under reduced pressure. The crude residue was dry loaded onto celite and purified by chromatography on RP Flash C18 (12 g cartridge, 0-50% (0.1% Formic acid in MeCN)/(0.1% Formic Acid in Water)) to afford the crude product as a brown colored solid (309 mg). The crude product was suspended in water (15 ml) and MeCN (10 mL) before conc. aq. HCl (1.04 g, 700 μL, 12 molar, 22 Eq, 8.40 mmol) was added, the suspension was stirred for 5 min to afford a solution. This was then concentrated to dryness and azeotroped with MeCN (2×10 mL) to afford (S)-5,5-dimethyl-2-((pyrimidin-4-ylmethyl)amino)hexanoic acid, HCl (100 mg, 0.33 mmol, 88%, 95% Purity) as an off-white solid.

UPLC (Method 4, 0.48 min; M+H=252.3. 1 H NMR (500 MHz, DMSO) δ 9.75 (brs, 2H), 9.27 (d, J=1.4 Hz, 1H), 8.88 (d, J=5.2 Hz, 1H), 7.71 (dd, J=5.1, 1.4 Hz, 1H), 4.42 (m, 2H), 4.07 (m, 1H), 2.00-1.83 (m, 2H), 1.39 (app.td, J=13.1, 4.9 Hz, 1H), 1.17 (app.td, J=12.8, 4.5 Hz, 1H), 0.87 (s, 9H).

### Example 137

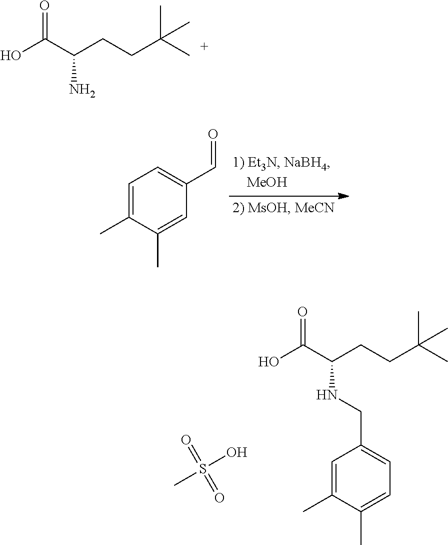

### Synthesis of (S)-2-((3,4-dimethylbenzyl)amino)-5,5-dimethylhexanoic acid, Mesylic acid

A suspension of (S)-2-amino-5,5-dimethylhexanoic acid (50 mg, 1 Eq, 0.31 mmol), 3,4-dimethylbenzaldehyde (42 mg, 1 Eq, 0.31 mmol) and Et₃N (32 mg, 44 μL, 1 Eq, 0.31 mmol) in MeOH (3 mL) was heated at 40° C. for 2 h before being cooled to 0° C. and treated with NaBH₄ (12 mg, 1 Eq, 0.31 mmol). The mixture was then allowed to warm to room temperature before concentrating to dryness. The mixture was then suspended in water (5 mL) and treated with acetic acid (42 mg, 40 μL, 2.2 Eq, 0.70 mmol) before filtering. The material was suspended in water (10 mL) and acetone (2 mL) before heating at 60° C. for 30 min then cooling and filtering. The freebase was suspended in MeCN (10 mL) and treated with 0.1 M MsOH in MeCN (1 eq) to afford a solution which was concentrated in vacuo. MeCN (2×5 mL) was added before the solvent was removed in vacuo to afford (S)-2-((3,4-dimethylbenzyl)amino)-5,5-dimethylhexanoic acid, Mesylic acid (69 mg, 0.18 mmol, 58%, 98% Purity) as a colourless solid.

LCMS (Method 5, 1.48 min; M+H=278.2. 1H NMR (500 MHz, DMSO) δ 14.02 (s, 1H), 9.16 (s, 2H), 7.25 (s, 1H), 7.22-7.14 (m, 2H), 4.08 (s, 2H), 3.90-3.84 (m, 1H), 2.30 (s, 3H), 2.24 (s, 3H), 2.24 (s, 3H), 1.89-1.71 (m, 2H), 1.32 (app. td, J=13.2, 4.7 Hz, 1H), 1.11 (app. td, J=13.0, 4.3 Hz, 1H), 0.85 (s, 9H).

### Example 138

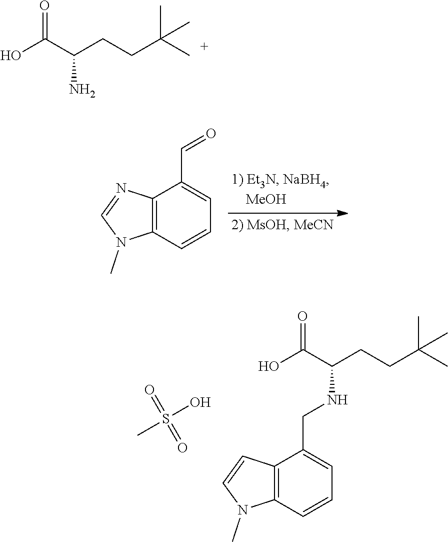

### Synthesis of (S)-5,5-dimethyl-2-(((1-methyl-1H-benzo[d]imidazol-4-yl)methyl)amino)hexanoic acid, Mesylic acid

A suspension of (S)-2-amino-5,5-dimethylhexanoic acid (100 mg, 1 Eq, 628 μmol), 1-methyl-1H-benzo[d]imidazole-4-carbaldehyde (101 mg, 1 Eq, 628 μmol) and Et₃N (64 mg, 88 μL, 1.0 Eq, 0.63 mmol) in MeOH (3 mL) was heated at 40° C. for 2 h before being cooled with an ice bath and treated with NaBH₄ (24 mg, 1.0 Eq, 0.63 mmol) in one portion. The mixture was then allowed to warm to rt before being concentrated to dryness. This was then suspended in water (5 mL) and acetic acid (84 mg, 80 μL, 2.2 Eq, 1.4 mmol) was added. This was diluted with MeCN (20 mL) then concentrated on to celite (˜2 g). The crude product was purified by chromatography on RP Flash C18 (12 g cartridge, 5-30% (0.1% Formic acid in MeCN)/(0.1% Formic Acid in Water)) to afford crude product which was suspended in MeCN (20 mL) and treated with 0.1 M MsOH in MeCN (1 eq) to afford a solution which was then concentrated to afford (S)-5,5-dimethyl-2-(((1-methyl-1H-benzo[d]imidazol-4-yl)methyl)amino)hexanoic acid, Mesylic acid (75 mg, 0.18 mmol, 28%, 95% Purity) as a colourless solid.

LCMS (Method 5, 0.97 min; M+H=304.2. 1H NMR (500 MHz, DMSO) δ 8.54 (s, 1H), 7.73 (d, J=7.5 Hz, 1H), 7.45-7.38 (m, 2H), 4.63-4.54 (m, 2H), 3.96 (dd, J=6.9, 4.7 Hz, 1H), 3.92 (s, 3H), 2.31 (s, 3H), 1.95-1.76 (m, 2H), 1.32 (app. td, J=13.1, 4.7 Hz, 1H), 1.10 (app. td, J=12.9, 4.4 Hz, 1H), 0.85 (s, 9H).

The following Examples were prepared in an analogous manner to Example 138, starting from the corresponding aldehyde.

| Example | Structure | Yield | UPLC | NMR |
|---|---|---|---|---|
| 139 | 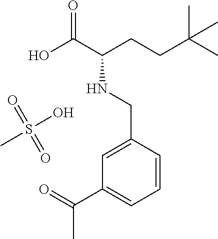 | 46 mg, (34% yield, 90% purity) | Method 5, 1.27 min, M + H = 292.2 | 1H NMR (500 MHz, DMSO) δ 8.06 (s, 1H), 7.98 (d, J = 7.7 Hz, 1H), 7.69 (d, J = 7.6 Hz, 1H), 7.57 (app. t, J = 7.7 Hz, 1H), 4.17-4.06 (m, 2H), 3.65-3.62 (m, 1H), 2.60 (s, 3H), 2.30 (s, 3H), 1.80-1.65 (m, 2H), 1.33-1.23 (m, 1H), 1.17 (app. td, J = 12.6, 5.0 Hz, 1H), 0.85 (s, 9H). |
| 140 | 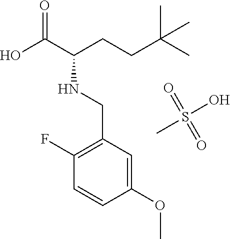 | 52 mg, (26% yield, 98% purity) | Method 5, 1.35 min, M + H = 298.2 | 1H NMR (500 MHz, DMSO) δ 14.08 (s, 1H), 9.32 (v br. s, 2H), 7.24 (app. t, J = 9.2 Hz, 1H), 7.16 (dd, J = 5.9, 3.2 Hz, 1H), 7.07-7.01 (m, 1H), 4.24-4.13 (m, 2H), 4.01- 3.97 (m, 1H), 3.78 (s, 3H), 2.30 (s, 3H), 1.94- 1.73 (m, 2H), 1.34 (app. td, J = 13.1, 4.7 Hz, 1H), 1.12 (app. td, J = 12.9, 4.3 Hz, 1H), 0.86 (s, 9H). 19F NMR (471 MHz, DMSO) δ −126.77. |

### Example 141 and 143

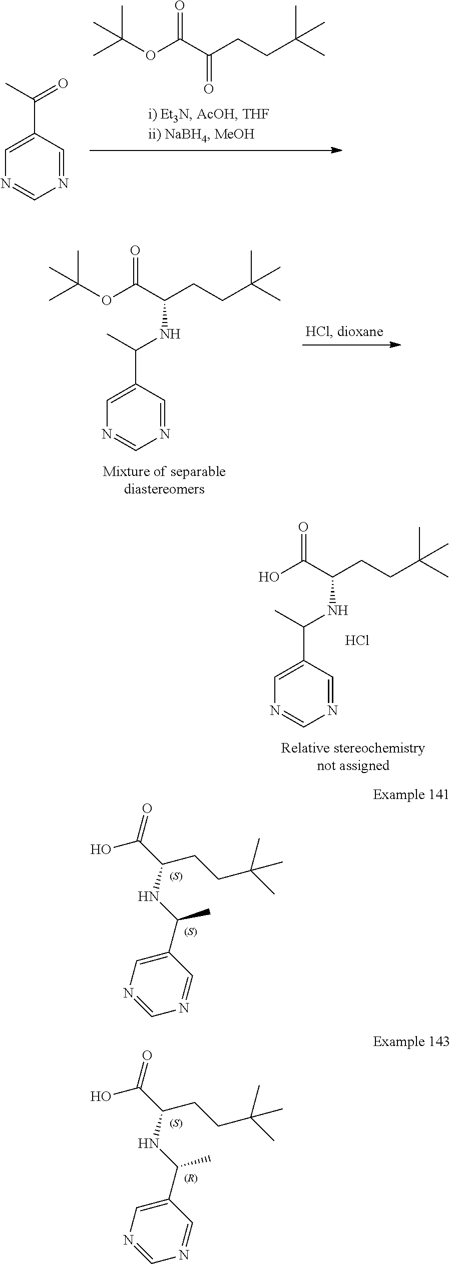

### Synthesis of (2-(((S)-1-(2-methoxypyridin-4-yl)ethyl)amino)-5,5-dimethylhexanoic acid, HCl

A solution of 1-(pyrimidin-5-yl)ethan-1-one (28 mg, 1 Eq, 0.23 mmol) and tert-butyl (S)-2-amino-5,5-dimethylhexanoate (50 mg, 1 Eq, 0.23 mmol) in THE (2 mL) was treated with Et₃N (23 mg, 32 μL, 1 Eq, 0.23 mmol) and AcOH (14 mg, 13 μL, 1 Eq, 0.23 mmol) before being heated at 50° C. for 2 h. The reaction mixture was concentrated to dryness and the residue was taken up in MeOH (1 mL) before being treated with NaBH₄ (10 mg, 1.1 Eq, 0.26 mmol). The mixture was stirred at room temperature for 1 hour before the solvent was removed in vacuo. The crude product was purified by chromatography on RP Flash C18 (12 g cartridge, 25-40% (0.1% Formic acid in MeCN)/(0.1% Formic Acid in Water)) to afford tert-butyl (2S)-5,5-dimethyl-2-((1-(pyrimidin-5-yl)ethyl)amino)hexanoate (21 mg, 65 μmol, 14%) as a colourless solid and tert-butyl (2S)-5,5-dimethyl-2-((1-(pyrimidin-5-yl)ethyl)amino)hexanoate (3 mg, 9 μmol, 2%) was isolated as a colourless gum.

Tert-butyl (2S)-5,5-dimethyl-2-((1-(pyrimidin-5-yl)ethyl)amino)hexanoate (21 mg, 7 Eq, 65 μmol) was treated with 4 M HCl in dioxane (4 mL) and stirred at 40° C. for 16 h before concentrating to dryness and triturating with MeCN (2 mL) to afford (2S)-5,5-dimethyl-2-((1-(pyrimidin-5-yl)ethyl)amino)hexanoic acid, HCl as a single diastereomer (15 mg, 49 μmol, 75%, 98% Purity) as a colourless solid.

LCMS (Method 5, 0.56 min; M+H=266.2. 1H NMR (500 MHz, DMSO) δ 14.03 (s, 1H), 10.14-9.71 (m, 2H), 9.23 (s, 1H), 9.02 (s, 2H), 4.56-4.51 (m, 1H), 3.66-3.57 (m, 1H), 1.92-1.83 (m, 1H), 1.80-1.72 (m, 1H), 1.68 (d, J=6.9 Hz, 3H), 1.24 (app. td, J=13.2, 4.7 Hz, 2H), 1.15-1.05 (m, 1H), 0.83 (s, 9H).

The other diastereomer was prepared via an analogous manner and isolated as a single diastereomer (3 mg, 9 μmol, 100%, 95% Purity) as a colourless solid.

LCMS (Method 5, 0.49 min; M+H=266.2. 1H NMR (500 MHz, DMSO) δ 9.17 (s, 1H), 8.93 (s, 2H), 4.33-4.22 (m, 1H), 3.75-3.65 (m, 1H), 1.87-1.68 (m, 2H), 1.59 (d, J=6.9 Hz, 3H), 1.32-1.04 (m, 2H), 0.85 (s, 9H).

### Example 142

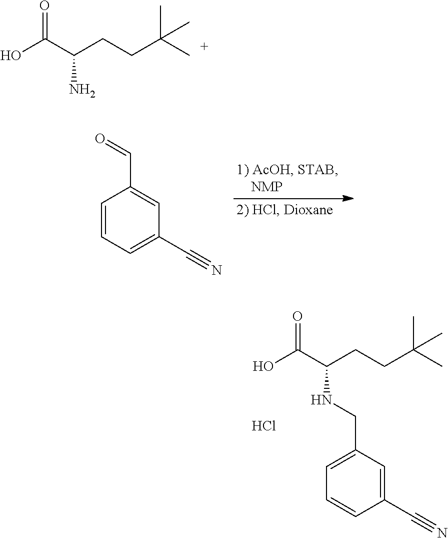 [SMILES: CC(C)(C)CC[C@H](N)C(=O)O ; N#Cc1cccc(C=O)c1 ; Cl ; CC(C)(C)CC[C@H](NCc1cccc(C#N)c1)C(=O)O]

### Synthesis of (S)-2-((3-cyanobenzyl)amino)-5,5-dimethylhexanoic acid, HCl

A vial was charged with (S)-2-amino-5,5-dimethylhexanoic acid (100 mg, 1 Eq, 628 μmol), 3-formylbenzonitrile (82 mg, 1.0 Eq, 0.63 mmol), acetic acid (38 mg, 36 μL, 1.0 Eq, 0.63 mmol) and NMP (3 mL). The suspension was stirred at rt for 2 h. Sodium triacetoxyborohydride (266 mg, 2 Eq, 1.26 mmol) was added to the mixture in one portion and the resultant mixture was stirred at rt for 20 h. The reaction was purified by SCX (˜1 g), first eluting with MeCN (30 mL) before eluting the product with NH₃ (7 M in MeOH)/MeCN (1:2, 100 mL). The ammoniacal fraction was concentrated under reduced pressure. The crude product was purified by chromatography on RP Flash C18 (12 g cartridge, 25-40% (0.1% Formic acid in MeCN)/(0.1% Formic Acid in Water)) to afford a white solid. This was dissolved in MeOH (1 mL) before being treated with 4 M HCl in dioxane (1 mL). The mixture was stirred at rt for 30 min before being concentrated to dryness to afford (S)-2-((3-cyanobenzyl)amino)-5,5-dimethylhexanoic acid, HCl (4 mg, 0.01 mmol, 70%, 98% Purity) as a colourless solid.

LCMS (Method 5, 1.23 min; M+H=275.0. 1H NMR (500 MHz, DMSO-d6) δ 7.97 (s, 1H), 7.91 (app. d, J=7.8 Hz, 1H), 7.82 (d, J=7.8 Hz, 1H), 7.67 (app. t, J=7.8 Hz, 1H), 4.22 (s, 2H), 4.01-3.91 (m, 1H), 1.92-1.74 (m, 2H), 1.36-1.27 (m, 1H), 1.16-1.07 (m, 1H), 0.87 (s, 9H).

### Examples 20-59 and 144

The following example compounds according to the invention were prepared:
Example 20: (2S)-5,5-dimethyl-2-({2-oxa-9-azatricyclo[9.4.0.0^{3,8}]pentadeca-1(11),3(8),4,6,9,12,14-heptaen-10-yl}amino)hexanoic acid
Example 21: (2S)-2-{[(1S)-2,2-difluoro-1-(3-methoxyphenyl)ethyl]amino}-5,5-dimethylhexanoic acid
Example 22: (2S)-2-{[(1R)-2,2-difluoro-1-(3-methoxyphenyl)ethyl]amino}-5,5-dimethylhexanoic acid
Example 23: (2S)-5,5-dimethyl-2-{[(1R)-2,2,2-trifluoro-1-(3-methoxyphenyl)ethyl]amino}hexanoic acid
Example 24: (2S)-5,5-dimethyl-2-{[(1S)-2,2,2-trifluoro-1-(3-methoxyphenyl)ethyl]amino}hexanoic acid
Example 25: (2S)-2-({[3-(hydroxymethyl)phenyl]methyl}amino)-5,5-dimethylhexanoic acid
Example 26: (2S)-2-{[(2,3-dimethoxyphenyl)methyl]amino}-5,5-dimethylhexanoic acid
Example 27: (2S)-2-{[(3,5-dimethoxyphenyl)methyl]amino}-5,5-dimethylhexanoic acid
Example 28: (2S)-2-{[(2,5-dimethoxyphenyl)methyl]amino}-5,5-dimethylhexanoic acid
Example 29: (2S)-2-{[(3-fluoro-5-methoxyphenyl)methyl]amino}-5,5-dimethylhexanoic acid
Example 30: (2S)-2-{[(3-chloro-5-methoxyphenyl)methyl]amino}-5,5-dimethylhexanoic acid
Example 31: (2S)-2-{[(3-bromo-5-methoxyphenyl)methyl]amino}-5,5-dimethylhexanoic acid
Example 32: (2S)-2-{[(3,5-dichlorophenyl)methyl]amino}-5,5-dimethylhexanoic acid
Example 33: (2S)-2-{[(3-methoxy-4-methylphenyl)methyl]amino}-5,5-dimethylhexanoic acid
Example 34: (2S)-2-{[(2-fluoro-3-methoxyphenyl)methyl]amino}-5,5-dimethylhexanoic acid
Example 35: (2S)-5,5-dimethyl-2-{[(quinolin-3-yl)methyl]amino}hexanoic acid
Example 36: (2S)-5,5-dimethyl-2-{[(quinolin-2-yl)methyl]amino}hexanoic acid
Example 37: (2S)-2-{[(3-fluoro-4-methoxyphenyl)methyl]amino}-5,5-dimethylhexanoic acid
Example 38: (2S)-2-{[(3,4-dimethoxyphenyl)methyl]amino}-5,5-dimethylhexanoic acid
Example 39: (2S)-5,5-dimethyl-2-{[(5,6,7,8-tetrahydronaphthalen-1-yl)methyl]amino}hexanoic acid
Example 40: (2S)-2-{[(3,4-dihydro-2H-1-benzopyran-6-yl)methyl]amino}-5,5-dimethylhexanoic acid
Example 41: (2S)-2-{[(2,3-dihydro-1,4-benzodioxin-6-yl)methyl]amino}-5,5-dimethylhexanoic acid
Example 42: (2S)-5,5-dimethyl-2-{[(quinoxalin-6-yl)methyl]amino}hexanoic acid
Example 43: (2S)-5,5-dimethyl-2-[({1H-pyrrolo[2,3-b]pyridin-5-yl}methyl)amino]hexanoic acid
Example 44: (2S)-5,5-dimethyl-2-[({1H-pyrrolo[2,3-b]pyridin-4-yl}methyl)amino]hexanoic acid
Example 45: (2S)-2-{[(2H-1,3-benzodioxol-4-yl)methyl]amino}-5,5-dimethylhexanoic acid
Example 46: (2S)-5,5-dimethyl-2-{[(quinolin-6-yl)methyl]amino}hexanoic acid
Example 47: (2S)-5,5-dimethyl-2-{[(quinolin-8-yl)methyl]amino}hexanoic acid
Example 48: (2S)-5,5-dimethyl-2-{[(quinolin-5-yl)methyl]amino}hexanoic acid
Example 49: (2S)-2-{[(2-methoxynaphthalen-1-yl)methyl]amino}-5,5-dimethylhexanoic acid
Example 50: (2S)-2-{[(1H-indol-2-yl)methyl]amino}-5,5-dimethylhexanoic acid
Example 51: (2S)-2-{[(1,3-benzothiazol-5-yl)methyl]amino}-5,5-dimethylhexanoic acid
Example 52: (2S)-5,5-dimethyl-2-{[(1-methyl-1H-pyrazol-5-yl)methyl]amino}hexanoic acid
Example 53: (2S)-2-{[(1,3-benzothiazol-6-yl)methyl]amino}-5,5-dimethylhexanoic acid
Example 54: (2S)-5,5-dimethyl-2-{[(1-methyl-1H-indazol-6-yl)methyl]amino}hexanoic acid
Example 55: (2S)-5,5-dimethyl-2-{[(pyrimidin-5-yl)methyl]amino}hexanoic acid
Example 56: (2S)-5,5-dimethyl-2-({[2-(pyridin-4-yl)phenyl]methyl}amino)hexanoic acid
Example 57: (2S)-2-({[3-(1H-imidazol-1-yl)phenyl]methyl}amino)-5,5-dimethylhexanoic acid
Example 58: (2S)-5,5-dimethyl-2-{[(pyridin-4-yl)methyl]amino}hexanoic acid
Example 59: (2S)-2-({[2-(hydroxymethyl)phenyl]methyl}amino)-5,5-dimethylhexanoic acid
Example 144: (2S)-2-{[(5-methoxypyridin-3-yl)methyl]amino}-5,5-dimethylhexanoic acid

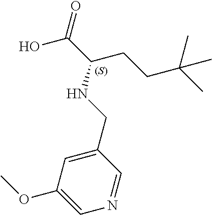

Any remaining example compounds were prepared in an analogous manner to the other examples.

### Biological Data

### Neurotensin Scintillation Proximity Assay

The exemplified compounds of the invention were tested in a Neurotensin (NTS) scintillation proximity assay (SPA). The IC₅₀ data is shown in Table 1 below. NTS, which is a 13 amino acid neuropeptide, is a sortilin ligand. The IC₅₀ is a measure of the amount of the compound required to inhibit the binding of NTS to sortilin by 50%. The skilled person will recognise that the lower the IC₅₀ value, the less of the compound needed to achieve the desired effect, and as a result, the chances of undesirable off-target effects are reduced.

Compound affinity was determined by measuring the displacement of [³H]-neurotensin binding to h-Sortilin in SPA format. Total volume of 40 μl in 50 mM HEPES pH 7.4 assay buffer containing 100 mM NaCl, 2.0 mM CaCl2, 0.1% BSA and 0.1% Tween-20. Compound pre-incubation for 30 minutes at room temperature with 150 nM of 6his-Sortilin before 5 nM [3H]-Neurotensin and Ni chelate imaging beads (Perkin Elmer) were added, after 6 hours the plate was read on a ViewLux with 360 s exposure time. Dose-response evaluation of compounds was performed with 8 concentrations of drugs (covering 3 decades). IC₅₀ values were calculated by nonlinear regression using the sigmoid concentration-response (variable slope) using CDD Vault software. All values reported are average of at least 2 determinations.

The data in Table 6 below shows that the compounds disclosed herein are sortilin inhibitors.

TABLE 1

| | IC₅₀ | |
|---|---|---|
| Representative | [3H]Neurotensin | |
| Examples | SPA | |
| Example 1 | 75 | nM |
| Example 3 | 766 | nM |
| Example 4 | 660 | nM |
| Example 5 | 560 | nM |
| Example 6 | 330 | nM |
| Example 7 | 950 | nM |
| Example 8 | 270 | nM |
| Example 9 | 630 | nM |
| Example 10 | 975 | nM |
| Example 11 | 1830 | nM |
| Example 12 | 2840 | nM |
| Example 13 | 1020 | nM |
| Example 14 | 610 | nM |
| Example 15 | 3480 | nM |
| Example 16 | 1480 | nM |
| Example 17 | 1610 | nM |
| Example 18 | 725 | nM |
| Example 19 | 565 | nM |
| Example 20 | 1490 | nM |
| Example 21 | 270 | nM |
| Example 22 | 260 | nM |
| Example 23 | 390 | nM |
| Example 25 | 1375 | nM |
| Example 28 | 2220 | nM |
| Example 29 | 550 | nM |
| Example 30 | 580 | nM |
| Example 31 | 520 | nM |
| Example 32 | 840 | nM |
| Example 33 | 400 | nM |
| Example 34 | 720 | nM |
| Example 35 | 650 | nM |
| Example 36 | 1600 | nM |
| Example 37 | 710 | nM |
| Example 38 | 1360 | nM |
| Example 39 | 360 | nM |
| Example 40 | 470 | nM |
| Example 41 | 330 | nM |
| Example 42 | 400 | nM |
| Example 43 | 470 | nM |
| Example 44 | 850 | nM |
| Example 45 | 1060 | nM |
| Example 46 | 920 | nM |
| Example 47 | 1950 | nM |
| Example 48 | 1060 | nM |
| Example 49 | 1990 | nM |
| Example 50 | 610 | nM |
| Example 51 | 320 | nM |
| Example 52 | 1560 | nM |
| Example 53 | 590 | nM |
| Example 54 | 870 | nM |
| Example 55 | 410 | nM |
| Example 56 | 1910 | nM |
| Example 57 | 1290 | nM |
| Example 58 | 810 | nM |
| Example 59 | 1280 | nM |
| Example 61 | 2830 | nM |
| Example 62 | >9980 | nM |
| Example 63 | 200 | nM |
| Example 64 | 460 | nM |
| Example 67 | 3310 | nM |
| Example 68 | 1200 | nM |
| Example 69 | 1390 | nM |
| Example 71 | 1130 | nM |
| Example 72 | 1240 | nM |
| Example 74 | 950 | nM |
| Example 75 | 980 | nM |
| Example 76 | 790 | nM |
| Example 77 | 600 | nM |
| Example 79 | 950 | nM |
| Example 80 | 790 | nM |
| Example 81 | 790 | nM |
| Example 83 | 700 | nM |
| Example 85 | 1050 | nM |
| Example 86 | 790 | nM |
| Example 87 | 530 | nM |
| Example 89 | 790 | nM |
| Example 91 | 390 | nM |
| Example 93 | 1630 | nM |
| Example 95 | 690 | nM |
| Example 100 | 380 | nM |
| Example 101 | 280 | nM |
| Example 102 | 540 | nM |
| Example 103 | 160 | nM |
| Example 106 | 590 | nM |
| Example 107 | 930 | nM |
| Example 108 | 370 | nM |
| Example 110 | 460 | nM |
| Example 112 | 280 | nM |
| Example 113 | 320 | nM |
| Example 114 | 220 | nM |
| Example 115 | 420 | nM |
| Example 116 | 600 | nM |
| Example 117 | 430 | nM |
| Example 125 | 550 | nM |
| Example 129 | 390 | nM |
| Example 131 | 380 | nM |
| Example 132 | 880 | nM |
| Example 133 | 820 | nM |
| Example 134 | 1320 | nM |
| Example 135 | 420 | nM |
| Example 136 | 1600 | nM |
| Example 137 | 740 | nM |
| Example 138 | 740 | nM |
| Example 139 | 570 | nM |
| Example 140 | 850 | nM |
| Example 141 | 640 | nM |
| Example 142 | 1260 | nM |
| Example 143 | 890 | nM |
| Example 144 | 430 | nM |

### Blood-Brain-Barrier Permeability

### Example 60—Plasma Protein Binding and Brain Homogenate Binding for Study Compounds in Mouse by Rapid Equilibrium Dialysis

To determine whether the compounds of the invention are capable of crossing the blood brain barrier, the Kₚᵤᵤ was calculated for Example 5, Example 6 and Comparative Example 1, which is a sortilin modulator that is not in accordance with the invention and has the following structure:

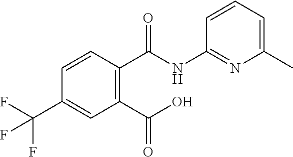 [SMILES: Cc1cccc(NC(=O)c2ccc(C(F)(F)F)cc2C(=O)O)n1]

Mice were dosed with the compounds of Example 5, Example 6 and Comparative Example 1, and then the plasma and brain were removed at specific timepoints to be analysed for compound concentration. Separately, the fraction of compound bound to plasma protein or brain homogenate was measured to allow assessment of free fractions.

The free drug hypothesis states that only unbound compound is able to permeate through biological membranes, interact with and elicit a pharmacological effect. Therefore, it is desirable for compounds to have a high free brain concentration. However, only the free unbound drug fraction is subject to Clearance mechanisms.

In practice, unbound fractions in plasma and brain tissue were assessed using rapid equilibrium dialysis in vitro. Separately a pharmacokinetic study was run in vivo whereby a dose of the compound of interest was given at T=0 hours and at subsequent timepoints (eg 0.5, 1 and 4 hours) plasma and separately brain samples were analysed for total concentration of the compound of interest. These total concentrations could then be adjusted with the unbound fraction to give the unbound concentrations in the plasma and brain. The unbound partition coefficient (Kₚᵤᵤ) was then determined as a ratio between the free compound concentrations in the compartment of interest, here the brain/CNS and the plasma.

Rapid Equilibrium Dialysis The test compounds were incubated in CD1-mouse plasma and brain homogenate at 37° C. in RED device with inserts (8K MWCO, Thermo scientific) for 4 hours at 5 μM in triplicates. 350 μL of 150 mM phosphate buffered saline (PBS, pH 7.4) was used as the receiver side solutions. The samples were collected from both sides after 4 hours equilibration time and their matrices were made similar by diluting the donor side sample using blank PBS, and by diluting the receiver side sample using blank plasma/phosphate buffered saline. After the incubation, aliquots of donor side matrixes were diluted with an equal volume of the blank receiver side matrix and aliquots of receiver side matrixes were diluted with an equal volume of blank donor side matrix. All samples were protein precipitated by addition of a two-fold volume of acetonitrile containing 100 nM of repaglinide as an internal standard. After 10-minute centrifugation at 13 200 rpm, the sample supernatants were analysed with an LC-MS/MS, to obtain the unbound fraction of the test compound (F_{ub}). The unbound fraction was calculated from the peak area ratios obtained for each matrix:

$${F}_{u b} = {C}_{P B S} / {C}_{p l a s m a} ;$$

where C_{PBS} and Cₚₗₐₛₘₐ are the analyte concentrations in PBS (receiver) and plasma (donor), respectively.

Recovery samples were prepared in each condition but without dialysis, and were used for evaluation of recovery from the dialysis experiment using following formula:

$$\% Recovery = 100 \times ( {V}_{P B S} \times {C}_{P B S} + {V}_{plasma} \times {C}_{plasma} ) / {V}_{plasma} \times {C}_{r e c o v e r y}$$

where V_{PBS} is the volume on the receiver side (PBS) and Vₚₗₐₛₘₐ is the volume on donor side (plasma) of the dialysis device. C_{recovery} is the analyte concentration measured from the recovery sample.

Propranolol (1 μM) and fluoxetine (5 μM) were included in the experiment as a control compounds.

The unbound fraction in brain (F_{ub, brain}) was calculated from the measured value in brain homogenate (F_{ub, meas}), taking into account the dilution factor used in preparing the brain homogenate:

$${F}_{ub , brain} = \frac{1 / D}{( \frac{1}{{F}_{ub , meas}} ) - 1 + ( \frac{1}{D} )}$$

where D=dilution factor (here 5).

### Analytical Method

Instrumentation: Waters Acquity UPLC+Waters Xevo TQ-XS triple quadrupole MS; Column: Waters Acquity HSS T3 (2.1×50 mm, 1.8 μm) column with pre-column filter; Gradient Elution; A=0.1% Formic acid, B=Acetonitrile

| Time (min) | Flow | A % | B % | Curve |
|---|---|---|---|---|
| 0.000 | 0.500 ml/min | 95 | 5 | — |
| 0.500 | 0.500 ml/min | 95 | 5 | 6 |
| 2.500 | 0.500 ml/min | 25 | 75 | 6 |
| 3.500 | 0.500 ml/min | 2 | 98 | 1 |
| 4.500 | 0.500 ml/min | 95 | 5 | 1 |

Temperature: 40° C.; Injection Volume: 1.5 μl; Ion Source: ESI+; Capillary voltage: 2400 V; Source temperature: 150° C.; Desolvation temperature: 650° C.; Cone gas flow: 240 L/hr; Desolvation gas flow: 1200 L/hr; Nebuliser gas flow: 7 Bar; Collision gas flow: 0.15 mL/min; Software: MassLynx 4.2

| | MRM | Collision | Cone | Retention |
|---|---|---|---|---|
| Compound | transition | energy (V) | (V) | time |
| Example 5 | 280 > 65 | 48 | 38 | 1.81 |
| | 280 > 91 | 34 | 38 | |
| | 280 > 121* | 16 | 38 | |
| Example 6 | 303 > 77 | 50 | 22 | 1.90 |
| | 303 > 144* | 18 | 22 | |
| Comparative | 325 > 92 | 32 | 46 | 2.11 |
| Example 1 | 325 > 109* | 16 | 46 | |
| | 325 > 217 | 26 | 64 | |
| Propranolol | 260 > 116* | 18 | 50 | 1.78 |
| | 260 > 155 | 23 | 50 | |
| | 260 > 183 | 25 | 50 | |
| Fluoxetine | 310 > 148* | 6 | 30 | 2.03 |

*MRM trace used for quantification

### Results of Equilibrium Dialysis

The table below shows, for the Mouse, the unbound fractions of compounds Example 5, Example 6 and the Comparative Example 1 in plasma and brain homogenate.

| | Mouse Plasma | Mouse Brain |
|---|---|---|
| | Unbound (Fu, %) | Unbound (Fu, %) |
| Example 5 | 3.3 | 29.1 |
| Example 6 | 5.8 | 22.9 |
| Comparative Example 1 | 6.8 | 27.3 |

### Example 21—Blood-Brain-Barrier Permeability of Study Compounds in Mice after PO Administration

The compound is administered to the animal in a suitable vehicle at T=0 hours. At one hour post administration plasma and separately brain are removed, prepared and analysed for total compound concentration.

### Sample Preparation—Brain

Mouse brain samples were prepared for analysis by homogenization with Omni bead ruptor, using 4-fold volume of 150 mM phosphate buffered saline (PBS) (E.g. 400 uL PBS+100 mg brain). After homogenization, a 30 μL homogenate sample was mixed with 60 μL of internal standard solution (100 ng/ml of repaglinide and phenacetin in acetonitrile (ACN) containing 1% formic acid) and mixed. Samples were centrifuged for 20 minutes (4000 rpm, Thermo Scientific SL16) and 50 μl of supernatant, together with 100 μl of 50% acetonitrile was transferred on an analytical plate. Standard samples were prepared by spiking blank brain homogenate to obtain concentrations from 0.1 to 10 000 ng/ml in brain homogenate by using one volume of spiking solution and nine volumes of blank homogenate. Quality control (QC) samples were prepared for concentrations at 3, 30, 300 and 3000 ng/ml by using one volume of spiking solution and nine volumes of blank brain homogenate. The standards and QCs were then prepared for analysis similarly as the samples. Blank brain matrix was collected in-house from CD-1 mice.

### Sample Preparation—Plasma

The samples were prepared by mixing 30 μL of plasma sample with 60 μL of internal standard solution (100 ng/ml of repaglinide and phenacetin in ACN with 1% of formic acid) and mixed. Samples were centrifuged for 20 minutes (4000 rpm, Thermo Scientific SL16) and 50 μl of supernatant, together with 100 μl of 50% acetonitrile was transferred on analytical plate. 10 μl of diluted samples were transferred on analytical plate and further diluted with 190 μl of 50% acetonitrile. Standard samples were prepared by spiking blank plasma to obtain concentrations from 0.1 to 10 000 ng/ml in plasma by using one volume of spiking solution and nine volumes of blank plasma. Quality control (QC) samples were prepared for concentrations at 3, 30, 300 and 3000 ng/ml by using one volume of spiking solution and nine volumes of plasma. The standards and QCs were then prepared for analysis similarly as the samples. Blank plasma was collected in-house from CD-1 mice.

For samples of the compound of Example 5 collected 15 min-4 h after dosing, additional 10-fold dilution was performed due to unexpectedly high concentrations (5 μl of analysed sample+45 μl of precipitated blank plasma).

### Analytical Method

Instrumentation: Waters Acquity UPLC+Waters TQ-S triple quadrupole MS; Column: Waters Acquity HSS T3 (2.1×50 mm, 1.8 μm) column with pre-column filter; Gradient Elution; A=0.1% Formic acid, B=Acetonitrile

| Time (min) | Flow | A % | B % | Curve |
|---|---|---|---|---|
| 0.000 | 0.500 ml/min | 95 | 5 | — |
| 0.500 | 0.500 ml/min | 95 | 5 | 6 |
| 2.500 | 0.500 ml/min | 25 | 75 | 6 |
| 3.500 | 0.500 ml/min | 2 | 98 | 1 |
| 4.500 | 0.500 ml/min | 95 | 5 | 1 |

Temperature: 40° C.; Injection Volume: 1.5 μl for brain, 4 μl for plasma; Ion Source: ESI+; Capillary voltage: 3000 V; Source temperature: 150° C.; Desolvation temperature: 650° C.; Cone gas flow: 220 L/hr; Desolvation gas flow: 1200 L/hr; Nebuliser gas flow: 7 mL/min; Collision gas flow: 0.15 mL/min; Software: MassLynx 4.2

| | | Collision | Cone | Retention |
|---|---|---|---|---|
| Compound | MRM transition | energy (V) | (V) | time |
| Example 5 | 280 > 65 | 44 | 38 | 1.77 |
| | 280 > 91 | 34 | | |
| | 280 > 121* | 16 | | |
| | 280 > 234 | 10 | | |
| Example 6 | 303 > 77* | 50 | 20 | 1.86 |
| | 303 > 103 | 42 | | |
| | 303 > 115 | 44 | | |
| | 303 > 144* | 14 | | |
| Phenacetin (IS)** | 180 > 110 | 17 | 20 | 1.86 |
| Repaglinide (IS)*** | 453 > 230 | 35 | | 2.35 |

*MRM used for quantification
**Used as an internal standard in quantification of brain homogenate samples
***Used as an internal standard in quantification of plasma samples

### Results

The results show that Example 5 and 6 have Kₚᵤᵤ greater than 0.1 and permeate the blood brain barrier. Comparative Example 1 had a Kₚᵤᵤ less than 0.1 and did not show a blood brain barrier penetration.

| | Mouse Plasma | | | Mouse Brain | | | |
|---|---|---|---|---|---|---|---|
| | | Total Plasma | Unbound Plasma | | Brain Tissue | Unbound Brain | |
| | | Concentration | Concentration | | Concentration | Concentration | |
| | Unbound | (ng/ml) | (ng/mL) | Unbound | (ng/g) | (ng/mL) | |
| | (Fu, %) | T = 1 hour | T = 1 hour | (Fu, %) | T = 1 hour | T = 1 hour | Kₚᵤᵤ |
| Example 5 | 3.3 | 3556 | 117 | 29.1 | 48.5 | 14 | 0.12 |
| Example 6 | 5.8 | 138.5 | 8 | 22.9 | 6.25 | 1.4 | 0.19 |
| Comparative | 6.8 | 393 | 27 | 27.3 | 0.9 | 0.2 | 0.00 |
| Example 1 | | | | | | | |

The unbound partition coefficient (Kₚᵤᵤ) was determined as a ratio between the free compound concentrations in plasma and brain:

$${K}_{p u u} = \frac{{C}_{ub , brain}}{{C}_{ub , plasma}}$$

Where C_{u,brain}=unbound concentration in brain (C×F_{ub, brain}); wherein
C=concentration at steady state; and
C_{ub,plasma}=unbound concentration in plasma (C×F_{ub}).

For the treatment of CNS diseases, it is desirable for the Kₚᵤᵤ to have a value more than 0.1, which indicates that a fraction of the unbound compound in the plasma permeates through the blood brain barrier.

### Example 146

### Creoptix (GCI) Method—Using SEQ ID NO. 4 (Murine Sortillin)

The GCI assay is based upon understood surface plasmon resonance methodologies specifically enhanced for detecting the binding of small entities to proteins. A protein, bound to a surface, is bathed in a solution containing potential ligands giving rise to binding kinetics to generate Kₒₙ and K_{off} rates, as well as a K_{d}. This methodology does not require the use of additional tracers and can be used with or without the element of competition with known ligands.

### Reagents:

| Reagent | Part no | Supplier |
|---|---|---|
| DMSO | D8418 | Sigma |
| Borate | B BELU-50 | Xantec Bioanalytics |
| EDTA | E7889-100ML | Merck Life Science UK |
| HBS-N | BR-1006-70 | Cytiva |
| 4PCH wave chip | 4PCH | Creoptix AG |
| rhSortilin | 3154-ST-050 | Bio-Techne Ltd |
| rmSortilin | 2934-ST-050 | Bio-Techne Ltd |
| EDC | BR100050 | Cytiva |
| NHS | BR100050 | Cytiva |
| Acetate pH 5.0 | BR100351 | Cytiva |
| Trizma/ Tris | 93352-1KG | Merck Life Science UK |

All buffers described were filtered using a 0.2 μm filter (Product number: 10300461, Nalgene) and degassed for 15 minutes prior to use.

A flow cell temperature of 25 degrees Celsius was used throughout the experiment.

### Chip Conditioning and Immobilisation

A 4PCH chip was conditioned across all flow cells using 0.2× concentration running buffer (running buffer composition: 1×HBS-N, pH 7.4, 3.4 mM EDTA, 1% DMSO) using pre-set conditioning wizard (WAVE control software) injecting: 0.1 M borate, 1 M NaCl (pH 9) followed by three start-up injections of 0.2× running buffer.

A buffer exchange was performed and 1× running buffer (1×HBS-N, pH 7.4, 3.4 mM EDTA, 1% DMSO) used during the immobilisation procedure:

An initial injection of EDC/NHS (mixed in 1:1 ratio) was performed across all 4-flow cells to activate the surface for amine coupling of ligands.

Recombinant Sortilin aliquots were thawed quickly by hand and centrifuged at 13,300 rpm for 10 minutes. A 10 μg/ml protein solution was then made in pH 5.0 acetate and injected once for 20 minutes over flow-cells 2, 3 and 4 for human, human (flow cells 2 & 3), and mouse Sortilin respectively followed by a 60 second dissociation period.

A final 7-minute passivation injection of 50 mM tris was used across all flow cells.

A flow rate of 10 μl/min was used for all conditioning and immobilisation cycles.

### Rapid Kinetics: Intermediate Binders

Compounds were screened at 1 μM using the in-built ‘intermediate binders’ settings: 100 ul/min, 45 s baseline, 25 s association, 300 s dissociation, with blanks every 5th sample and a DMSO correction (1.5% DMSO at the start and end of the experiment as well as every 20 cycles. An acquisition rate of 10 Hz was used throughout the experiment.

Compounds were screened at a final assay concentration of 1 μM and a final DMSO concentration was 1%. To achieve this, compounds were diluted in DMSO from 10 mM stocks to 100 μM (100×final assay concentration) then diluted 1:100 in running buffer which contained no DMSO to establish a final assay concentration of compound of 1 μM and a final DMSO concentration of 1% [DMSO].

Compounds and DMSO were mixed by plate shaking at 1000 rpm for 60 seconds using a Bioshake instrument.

A flow rate of 100 μl/min was used throughout the experiment.

### Data Evaluation:

Data was evaluated using the RAPID kinetic analysis tool in the GCI WAVE_control software with data fitted using a standard 1:1 kinetic BioModel.

| Example | | | KD (M) |
|---|---|---|---|
| No | Form | Species | Sortilin |
| 3 | HCl salt | Human | 9.02E−07 |
| 5 | Free acid | Human | 1.93E−06 |
| 5 | HCl salt | Mouse | 1.02E−06 |
| 5 | Ms salt | Human | 4.23E−07 |
| 5 | MsOH salt | Human | 3.96E−07 |
| 9 | HCl salt | Human | 8.45E−06 |
| 21 | Free acid | Human | 2.84E−07 |
| 22 | Free acid | Human | 5.96E−06 |
| 23 | Free acid | Human | 4.02E−06 |
| 24 | Free acid | Human | 2.30E−07 |
| 28 | Free acid | Human | 1.02E−06 |
| 29 | Free acid | Human | 5.71E−07 |
| 30 | Free acid | Human | 4.50E−07 |
| 31 | Free acid | Human | 4.32E−07 |
| 33 | Free acid | Human | 2.42E−07 |
| 34 | Free acid | Human | 4.92E−07 |
| 35 | Free acid | Human | 8.95E−07 |
| 35 | Free acid | Mouse | 1.05E−06 |
| 38 | Free acid | Human | 7.47E−07 |
| 40 | Free acid | Human | 1.01E−07 |
| 41 | Free acid | Human | 5.96E−06 |
| 45 | Free acid | Human | 2.85E−07 |
| 46 | Free acid | Human | 6.96E−07 |
| 46 | Free acid | Mouse | 7.87E−07 |
| 55 | Free acid | Human | 1.98E−06 |
| 63 | Free acid | Human | 5.67E−08 |
| 64 | Free acid | Human | 1.02E−07 |
| 69 | Free acid | Human | 2.76E−06 |
| 74 | Free acid | Human | 3.39E−06 |
| 75 | Free acid | Human | 4.43E−07 |
| 101 | Free acid | Human | 2.77E−07 |
| 102 | Free acid | Human | 4.07E−07 |
| 103 | Free acid | Human | 8.32E−08 |
| 107 | Free acid | Human | 4.43E−07 |
| 112 | Free acid | Human | 1.83E−07 |
| 113 | Free acid | Human | 6.25E−07 |
| 114 | Free acid | Human | 1.32E−07 |
| 115 | Free acid | Human | 3.51E−07 |
| 125 | Ms salt | Human | 5.22E−07 |
| 129 | Ms salt | Human | 5.12E−07 |
| 131 | Free acid | Human | 9.76E−08 |
| 136 | Free acid | Human | 2.64E−05 |
| 137 | Free acid | Human | 2.08E−07 |
| 140 | Free acid | Human | 4.56E−07 |
| 144 | Free acid | Human | 3.10E−07 |

For compounds of the invention it is advantageous to have Kd lower than 1.00 E-4. The K_{d} data generated demonstrate that examples of the invention bind directly to the sortilin protein and bind in a generally similar manner in both human and murine sortilin protein which is advantageous for the development of non-human models of disease.

### REFERENCES

Andersen, J et al., Identification of the first small-molecule ligand of the neuronal receptor sortilin and structure determination of the receptor-ligand complex. Acta Crystallogr D Biol Crystallogr (2014), 70(Pt 2), pp. 451-460;
Baker, M. et al., Mutations in progranulin cause tau-negative frontotemporal dementia linked to chromosome 17. Nature (2006), 442(7105), pp. 916-919;
Brouwers, N. et al., Genetic variability in progranulin contributes to risk for clinically diagnosed Alzheimer disease. Neurology, (2008), 71(9), pp. 656-664;
Buttenshon, H. N. et al., Increased serum levels of sortilin are associated with depression and correlated with BDNF and VEGF, Nature Translational Psychiatry (2015), 5(e677), pp. 1-7;
Carecchio, M., et al., Cerebrospinal fluid biomarkers in Progranulin mutations carriers. J Alzheimers Dis (2011), 27(4), pp. 781-790;
Carrasquillo, M. et al., Genome-wide screen identifies rs646776 near sortilin as a regulator of progranulin levels in human plasma. Am J Hum Genet (2010), 87(6), pp. 890-897;
Chen, Z. Y. et al., Sortilin controls intracellular sorting of brain-derived neurotrophic factor to the regulated secretory pathway. J Neurosci (2005), 25(26), pp. 6156-6166;
Cruts, M. et al., Loss of progranulin function in frontotemporal lobar degeneration. Trends Genet (2008), 24(4), pp. 186-194;
De Muynck, L. et al., The neurotrophic properties of progranulin depend on the granulin E domain but do not require sortilin binding. Neurobiol Aging (2013), 34(11), pp. 2541-2547;
Egashira, Y. et al., The growth factor progranulin attenuates neuronal injury induced by cerebral ischemia-reperfusion through the suppression of neutrophil recruitment. J Neuroinflammation (2013), 10, pp. 105;
Galimberti, D. et al., GRN variability contributes to sporadic frontotemporal lobar degeneration. J Alzheimers Dis (2010), 19(1), pp. 171-177;
Galimberti, D. et al., Progranulin as a therapeutic target for dementia. Expert Opin Ther Targets (2018), 22(7), pp. 579-585. doi:10.1080/14728222.2018.1487951
Gao, A. et al., Implications of Sortilin in Lipid Metabolism and Lipid Disorder Diseases. DNA and Cell Biology (2017), 36(12), pp. 1050-1061;
Gass, J. et al., Progranulin regulates neuronal outgrowth independent of sortilin. Mol Neurodegener (2012), 7, pp. 33;
Gass, J. et al., Progranulin: an emerging target for FTLD therapies. Brain Res (2012), 1462, pp. 118-128;
Gijselinck, I., et al., Granulin mutations associated with frontotemporal lobar degeneration and related disorders: an update. Hum Mutat (2008), 29(12), pp. 1373-1386;
Goettsch, C., et al., Sortilin and Its Multiple Roles in Cardiovascular and Metabolic Diseases. Atherosclerosis, Thrombosis and Vascular Biology (2017), 38(1), pp. 19-25
Jansen, P., et al., Roles for the pro-neurotrophin receptor sortilin in neuronal development, aging and brain injury. Nature Neuroscience (2007), 10(11), pp. 1449-1457;
Hu, F. et al., Sortilin-mediated endocytosis determines levels of the frontotemporal dementia protein, progranulin. Neuron (2010), 68(4), pp. 654-667;
Huang, G. et al., Insulin responsiveness of glucose transporter 4 in 3T3-L1 cells depends on the presence of sortilin. Mol Biol Cell(2013), 24(19), pp. 3115-3122;
Kaddai, V. et al. Involvement of TNF-α in abnormal adipocyte and muscle sortilin expression in obese mice and humans. Diabetologia (2009) 52, pp. 932-940;
Kjolby, M. et al., Sort1, encoded by the cardiovascular risk locus 1p13.3, is a regulator of hepatic lipoprotein export. Cell Metab (2010), 12(3), pp. 213-223;
Laird, A. S. et al., Progranulin is neurotrophic in vivo and protects against a mutant TDP-43 induced axonopathy. PLoS One (2010), 5(10), e13368;
Lee, W. et al., Targeted manipulation of the sortilin-progranulin axis rescues progranulin haploinsufficiency. Hum Mol Genet (2014), 23(6), pp. 1467-1478;
Martens, L. et al., Progranulin deficiency promotes neuroinflammation and neuron loss following toxin-induced injury. J Clin Invest (2012), 122(11), pp. 3955-3959;
Mazella, J. et al., The 100-kDa neurotensin receptor is gp95/sortilin, a non-G-protein-coupled receptor. J Biol Chem (1998), 273(41), pp. 26273-26276;
Miyakawa, S. et al, Anti-sortilin1 Antibody Up-Regulates Progranulin via Sortilin1 Down-Regulation. Front Neurosci (2020), 14, pp. 586107;
Møller et al. Sortilin as a Biomarker for Cardiovascular Disease Revisited. Frontiers in Cardiovascular Medicine (2021), 8, 652584;
Mortensen, M. B. et al., Targeting sortilin in immune cells reduces proinflammatory cytokines and atherosclerosis. J Clin Invest (2014), 124(12), pp. 5317-5322;
Nykjaer, A et al., Sortilin is essential for proNGF-induced neuronal cell death. Nature (2014), 427(6977), pp. 843-848;
Nykjaer, A., & Willnow, T. E, Sortilin: a receptor to regulate neuronal viability and function. Trends Neurosci(2012), 35(4), pp. 261-270.
Oh, T. J. et al., Circulating sortilin level as a potential biomarker for coronary atherosclerosis and diabetes mellitus. Cardiovascular Diabetology (2017), 16(92);
Pan, X. et al., Sortilin and retromer mediate retrograde transport of Glut4 in 3T3-L1 adipocytes. Mol Biol Cell (2017), 28(12), pp. 1667-1675;
Petersen, C. et al., Molecular identification of a novel candidate sorting receptor purified from human brain by receptor-associated protein affinity chromatography. J Biol Chem (1997), 272(6), pp. 3599-3605;
Pickford, F. et al., Progranulin is a chemoattractant for microglia and stimulates their endocytic activity. Am J Pathol (2011), 178(1), pp. 284-295;
Pottier, C., et al., Potential genetic modifiers of disease risk and age at onset in patients with frontotemporal lobar degeneration and GRN mutations: a genome-wide association study. Lancet Neurol(2018), 17(6), pp. 548-558;
Quistgaard, E. et al., Ligands bind to Sortilin in the tunnel of a ten-bladed beta-propeller domain. Nat Struct Mol Biol(2009), 16(1), pp. 96-98;
Santos, A. M. et al., Sortilin Participates in Light-dependent Photoreceptor Degeneration in Vivo. PLoS ONE (2012), 7(4), pp. e36243-e36243.16.
Kuruvilla, R. et al., A neurotrophin signaling cascade coordinates sympathetic neuron development through differential control of TrkA trafficking and retrograde signalling. Cell (2004), 118(2), pp. 243-255;
Shi, J. & Kandror, K. V., Sortilin Is Essential and Sufficient for the Formation of Glut4 Storage Vesicles in 3T3-L1 Adipocytes. Developmental Cell (2005), 9, pp. 99-108;
Schroder, T. et al., The identification of AF38469: an orally bioavailable inhibitor of the VPS10P family sorting receptor Sortilin. Bioorg Med Chem Lett (2014), 24(1), pp. 177-180;
Sheng, J. et al., Progranulin polymorphism rs5848 is associated with increased risk of Alzheimer's disease. Gene (2014), 542(2), pp. 141-145; Skeldal, S. et al., Mapping of the Interaction Site between Sortilin and the p75 Neurotrophin Receptor Reveals a Regulatory Role for the Sortilin Intracellular Domain in p75 Neurotrophin Receptor Shedding and Apoptosis. J Biol Chem (2012), 21(287), pp. 43798-43809;
Tauris, J., et al., Proneurotrophin-3 May Induce Sortilin-Dependent Death In Inner Ear Neurons. Eur J Neuroscience (2020), 33(4), pp. 622-31;
Tang, W. et al., The growth factor progranulin binds to TNF receptors and is therapeutic against inflammatory arthritis in mice. Science (2011), 332(6028), pp. 478-484;
Tao, J. et al., Neuroprotective effects of progranulin in ischemic mice. Brain Res (2012), 1436, pp. 130-136;
Tenk, H. K., et al., ProBDNF induces neuronal apoptosis via activation of a receptor complex of p75NTR and sortilin. J Neuroscience (2005), 10(11), pp. 1449-1457
Van Kampen, J. M., et al., Progranulin gene delivery protects dopaminergic neurons in a mouse model of Parkinson's disease. PLoS One (2014), 9(5), e97032;
Willnow, T. E. et al., VPS10P-domain receptors—regulators of neuronal viability and function. Nat Rev Neurosci(2008), 9(12), pp. 899-909;
Willnow, T. E., et al., Sortilins: new players in lipoprotein metabolism. Current Opinion in Lipidology (2011), 22(2), pp. 79-85.
Wuts, P. G. M. and Greene, T. W, Greene's Protective Groups in Organic Synthesis, 4th Edition, John Wiley and Sons, New York (2006);
Xu, S. H. et al., Regional and Cellular Mapping of Sortilin Immunoreactivity in Adult Human Brain, Frotiers in Neuroanatomy (2019), 13(31), pp. 1-27;
Yano, H., et al., Proneurotrophin-3 is a neuronal apoptotic ligand: evidence for retrograde-directed cell killing. J Neurosci (2009), 29(47), pp. 14790-14802;
Yin, F., et al., Exaggerated inflammation, impaired host defense, and neuropathology in progranulin-deficient mice. J Exp Med (2010), 207(1), pp. 117-128;
Zheng, Y., et al., C-terminus of progranulin interacts with the beta-propeller region of sortilin to regulate progranulin trafficking. PLoS One (2011), 6(6), e21023;
Zhou, X. et al., Prosaposin facilitates sortilin-independent lysosomal trafficking of progranulin. J Cell Biol (2015), 210(6), pp. 991-1002;
Meneses et al., TDP-43 Pathology in Alzheimer's Disease, Mol Neurodegeneration (2021), 16, 84;
Prudencio et al., Misregulation of human sortilin splicing leads to the generation of a nonfunctional progranulin receptor, Proc Natl Acad Sci USA (2012), 109(52): 21510-21515;
Beel et al., Progranulin reduces insoluble TDP-43 levels, slows down axonal degeneration and prolongs survival in mutant TDP-43 mice, Mol Neurodegener. (2018), 13: 55.

Sequences referenced throughout the specification and forming part of the description

```
(full length sortilin-isoform 1)
SEQ ID NO: 1
    1 MERPWGAADG LSRWPHGLGL LLLLQLLPPS TLSQDRLDAP PPPAAPLPRW
  51 SGPIGVSWGL RAAAAGGAFP RGGRWRRSAP GEDEECGRVR DFVAKLANNT
101 HQHVFDDLRG SVSLSWVGDS TGVILVLTTF HVPLVIMTFG QSKLYRSEDY
151 GKNFKDITDL INNTFIRTEF GMAIGPENSG KVVLTAEVSG GSRGGRIFRS
201 SDFAKNFVQT DLPFHPLTQM MYSPQNSDYL LALSTENGLW VSKNFGGKWE
251 EIHKAVCLAK WGSDNTIFFT TYANGSCKAD LGALELWRTS DLGKSFKTIG
301 VKIYSFGLGG RFLFASVMAD KDTTRRIHVS TDQGDTWSMA QLPSVGQEQF
351 YSILAANDDM VFMHVDEPGD TGFGTIFTSD DRGIVYSKSL DRHLYTTTGG
401 ETDFTNVTSL RGVYITSVLS EDNSIQTMIT FDQGGRWTHL RKPENSECDA
451 TAKNKNECSL HIHASYSISQ KLNVPMAPLS EPNAVGIVIA HGSVGDAISV
501 MVPDVYISDD GGYSWTKMLE GPHYYTILDS GGIIVAIEHS SRPINVIKFS
551 TDEGQCWQTY TFTRDPIYFT GLASEPGARS MNISIWGFTE SFLTSQWVSY
601 TIDFKDILER NCEEKDYTIW LAHSTDPEDY EDGCILGYKE QFLRLRKSSM
651 CQNGRDYVVT KQPSICLCSL EDFLCDFGYY RPENDSKCVE QPELKGHDLE
701 FCLYGREEHL TTNGYRKIPG DKCQGGVNPV REVKDLKKKC TSNFLSPEKQ
751 NSKSNSVPII LAIVGLMLVT VVAGVLIVKK YVCGGRFLVH RYSVLQQHAE
801 ANGVDGVDAL DTASHTNKSG YHDDSDEDLL E
(full length sortilin-isoform 2)
SEQ ID NO: 2
    1 MERPWGAADG LSRWPHGLGL LLLLQLLPPS TLSQDRLDAP PPPAAPLPRW
  51 SGPIGVSWGL RAAAAGGAFP RGGRWRRSAP GEDEECGRVR DFVAKLANNT
101 HQHVFDDLRG SVSLSWVGDS TGVILVLTTF HVPLVIMTFG QSKLYRSEDY
151 GKNFKDITDL INNTFIRTEF GMAIGPENSG KVVLTAEVSG GSRGGRIFRS
201 SDFAKNFVQT DLPFHPLTQM MYSPQNSDYL LALSTENGLW VSKNFGGKWE
251 EIHKAVCLAK WGSDNTIFFT TYANGSCTDL GALELWRTSD LGKSFKTIGV
301 KIYSFGLGGR FLFASVMADK DTTRRIHVST DQGDTWSMAQ LPSVGQEQFY
351 SILAANDDMV FMHVDEPGDT GFGTIFTSDD RGIVYSKSLD RHLYTTTGGE
401 TDFTNVTSLR GVYITSVLSE DNSIQTMITF DQGGRWTHLR KPENSECDAT
451 AKNKNECSLH IHASYSISQK LNVPMAPLSE PNAVGIVIAH GSVGDAISVM
501 VPDVYISDDG GYSWTKMLEG PHYYTILDSG GIIVAIEHSS RPINVIKFST
551 DEGQCWQTYT FTRDPIYFTG LASEPGARSM NISIWGFTES FLTSQWVSYT
601 IDFKDILERN CEEKDYTIWL AHSTDPEDYE DGCILGYKEQ FLRLRKSSVC
651 QNGRDYVVTK QPSICLCSLE DFLCDFGYYR PENDSKCVEQ PELKGHDLEF
701 CLYGREEHLT TNGYRKIPGD KCQGGVNPVR EVKDLKKKCT SNFLSPEKQN
751 SKSNSVPIIL AIVGLMLVTV VAGVLIVKKY VCGGRFLVHR YSVLQQHAEA
801 NGVDGVDALD TASHTNKSGY HDDSDEDLLE
(mature sortilin)
SEQ ID NO: 3
    1 MTFGQSKLYR SEDYGKNFKD ITDLINNTFI RTEFGMAIGP ENSGKVVLTA
  51 EVSGGSRGGR IFRSSDFAKN FVQTDLPFHP LTQMMYSPQN SDYLLALSTE
101 NGLWVSKNFG GKWEEIHKAV CLAKWGSDNT IFFTTYANGS CTDLGALELW
151 RTSDLGKSFK TIGVKIYSFG LGGRFLFASV MADKDTTRRI HVSTDQGDTW
201 SMAQLPSVGQ EQFYSILAAN DDMVFMHVDE PGDTGFGTIF TSDDRGIVYS
251 KSLDRHLYTT TGGETDFTNV TSLRGVYITS VLSEDNSIQT MITFDQGGRW
301 THLRKPENSE CDATAKNKNE CSLHIHASYS ISQKLNVPMA PLSEPNAVGI
361 VIAHGSVGDA ISVMVPDVYI SDDGGYSWTK MLEGPHYYTI LDSGGIIVAI
401 EHSSRPINVI KFSTDEGQCW QTYTFTRDPI YFTGLASEPG ARSMNISIWG
451 FTESFLTSQW VSYTIDFKDI LERNCEEKDY TIWLAHSTDP EDYEDGCILG
501 YKEQFLRLRK SSVCQNGRDY VVTKQPSICL CSLEDFLCDF GYYRPENDSK
551 CVEQPELKGH DLEFCLYGRE EHLTTNGYRK IPGDKCQGGV NPVREVKDLK
601 KKCTSNFLSP EKQNSKSNSV PIILAIVGLM LVTVVAGVLI VKKYVCGGRF
651 LVHRYSVLQQ HAEANGVDGV DALDTASHTN KSGYHDDSDE DLLE
(Murine Sortilin)
>sp|Q6PHU5|SORT_MOUSE Sortilin
OS = Mus musculus OX = 10090 GN = Sort1 PE = 1 SV = 1
SEQ ID NO: 4
MERPRGAADGLLRWPLGLLLLLQLLPPAAVGQDRLDAPPPPAPPLLRWAGPVGVSWGLRA
AAPGGPVPRAGRWRRGAPAEDQDCGRLPDFIAKLTNNTHQHVFDDLSGSVSLSWVGDSTG
VILVLTTFQVPLVIVSFGQSKLYRSEDYGKNFKDITNLINNTFIRTEFGMAIGPENSGKV
ILTAEVSGGSRGGRVFRSSDFAKNFVQTDLPFHPLTQMMYSPQNSDYLLALSTENGLWVS
KNFGEKWEEIHKAVCLAKWGPNNIIFFTTHVNGSCKADLGALELWRTSDLGKTFKTIGVK
IYSFGLGGRFLFASVMADKDTTRRIHVSTDQGDTWSMAQLPSVGQEQFYSILAANEDMVF
MHVDEPGDTGFGTIFTSDDRGIVYSKSLDRHLYTTTGGETDFTNVTSLRGVYITSTLSED
NSIQSMITFDQGGRWEHLRKPENSKCDATAKNKNECSLHIHASYSISQKLNVPMAPLSEP
NAVGIVIAHGSVGDAISVMVPDVYISDDGGYSWAKMLEGPHYYTILDSGGIIVAIEHSNR
PINVIKFSTDEGQCWQSYVFTQEPIYFTGLASEPGARSMNISIWGFTESFITRQWVSYTV
DFKDILERNCEEDDYTTWLAHSTDPGDYKDGCILGYKEQFLRLRKSSVCQNGRDYVVAKQ
PSVCPCSLEDFLCDFGYFRPENASECVEQPELKGHELEFCLYGKEEHLTTNGYRKIPGDK
CQGGMNPAREVKDLKKKCTSNFLNPTKQNSKSNSVPIILAIVGLMLVTVVAGVLIVKKYV
CGGRFLVHRYSVLQQHAEADGVEALDSTSHAKSGYHDDSDEDLLE
```

1. A compound that binds to and modulates the activity of sortilin, wherein the compound has a blood-to-brain Kₚᵤᵤ of more than 0.1.

2. A method of treatment or prevention of a disease of the central nervous system comprising administration of the compound of claim 1,
wherein the disease of the central nervous system is selected from:
a neurodegenerative disorder selected from motor neuron diseases, Frontotemporal Lobar Degeneration (FTLD), frontotemporal dementia, Alzheimer's disease, Parkinson's disease, Huntington's disease, prion diseases such as Creutzfeldt-Jakob disease (CJD), acute brain injury, spinal cord injury and stroke;
a psychiatric disorder selected from bipolar disorder, major depression, post-traumatic stress disorder, and anxiety disorders; or
hearing loss selected from noise-induced hearing loss, ototoxicity induced hearing loss, age-induced hearing loss, idiopathic hearing loss, tinnitus and sudden hearing loss; brain tumours, retinopathies, glaucoma, neuroinflammation, chronic pain and diseases characterized by misfolded tau.

3. A compound of formula (I) 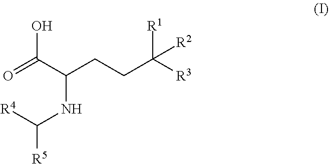
or a pharmaceutically acceptable salt, solvate, hydrate, tautomer, optical isomer, N-oxide, and/or prodrug thereof; wherein
R¹, R² and R³ are each independently selected from the group consisting of halo, H, (C₁-C₄)alkyl, halo-(C₁-C₄)alkyl, (C₂-C₄)alkenyl, and halo-(C₂-C₄)alkenyl; and
R⁴ is selected from the group consisting of H, (C₁-C₃)alkyl, halo-(C₁-C₃)alkyl, (C₃-C₈)aryl, halo-(C₃-C₈)aryl, (C₃-C₈)heteroaryl and halo-(C₃-C₈)heteroaryl;
R⁵ is selected from the group consisting of (C₃-C₂₀)-aryl, (C₃-C₂₀)-heteroaryl and 3- to 12-membered-heterocyclic ring;
wherein the aryl, heteroaryl or heterocyclic ring is optionally substituted with one or more substituents independently selected from halo, —OH, cyano, carbonyl, (C₁-C₄)alkyl, (C₁-C₄)hydroxyalkyl, halo-(C₁-C₄)alkyl, acetyl, (C₁-C₄)alkoxy, halo-(C₁-C₄)alkoxy (C₃-C₈)aryl and (C₃-C₈)heteroaryl; or
R⁴ and R⁵ taken together form a 6- to 20-membered-heterocyclic ring;
wherein the heterocyclic ring is monocyclic, bicyclic or tricyclic and is optionally substituted with one or more substituents independently selected from halo, —OH, cyano, carbonyl, (C₁-C₄)alkyl, halo-(C₁-C₁₀)alkyl, acetyl, (C₁-C₄)alkoxy, and halo-(C₁-C₄)alkoxy.

4. The compound according to claim 3, wherein R¹, R² and R³ are each independently selected from the group consisting of halo, (C₁-C₂)alkyl and halo-(C₁-C₂)alkyl.

5. The compound according to claim 3, wherein R¹, R² and R³ are each independently selected from F, CH₃ and CF₃.

6. The compound according to claim 3, wherein R⁴ is selected from the group consisting of H, (C₁-C₂)alkyl, halo-(C₁-C₂)alkyl, (C₅-C₈)aryl, halo-(C₅-C₈)aryl, (C₃-C₈)heteroaryl and halo-(C₃-C₈)heteroaryl.

7. The compound according to claim 6, wherein R⁴ is selected from the group consisting of:
(i) H (ii) CH₃ (iii) CF₃
(iv) CHF₂ and

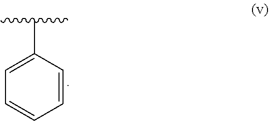 [SMILES: *c1ccccc1]

8. The compound according to claim 3, wherein R⁵ is selected from the group consisting of (C₅-C₁₂)-aryl, (C₅-C₁₂)-heteroaryl and 5- to 12-membered-heterocyclic ring;
wherein the aryl, heteroaryl or heterocyclic ring is optionally substituted with one or more substituents independently selected from halo, —OH, cyano, carbonyl, (C₁-C₂)alkyl, (C₁-C₂)hydroxyalkyl, halo-(C₁-C₂)alkyl, (C₁-C₂)alkoxy, halo-(C₁-C₂)alkoxy (C₃-C₈)aryl and (C₃-C₈)heteroaryl.

9. The compound according to claim 8, wherein R⁵ is selected from the group consisting of:

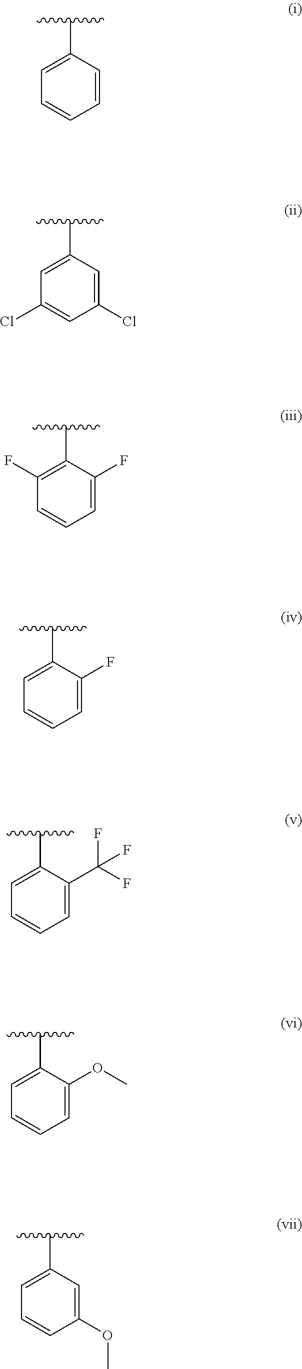

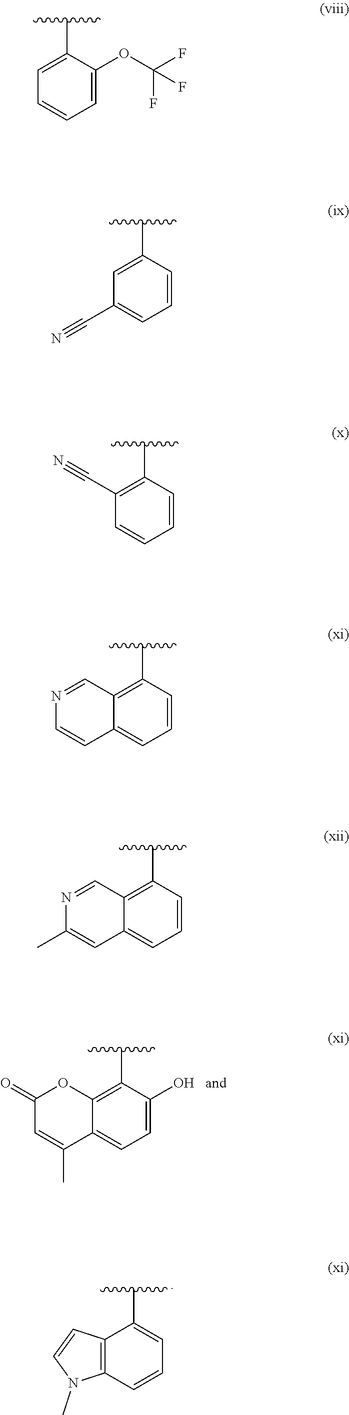

10. The compound according to claim 3, wherein R⁴ and R⁵ taken together form an 8- to 20-membered-heterocyclic ring;
wherein the heterocyclic ring is tricyclic.

11. The compound according to claim 10, wherein R⁴ and R⁵ taken together form the following structure:

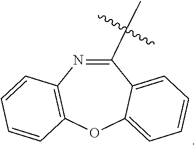 [SMILES: *C1=Nc2ccccc2Oc2ccccc21]

12. The compound according to claim 3, wherein the compound of Formula (I) is:
(S)-2-(((7-hydroxy-4-methyl-2-oxo-2H-chromen-8-yl)methyl)amino)-5,5-dimethylhexanoic acid;
rac-2-(((7-hydroxy-4-methyl-2-oxo-2H-chromen-8-yl)methyl)amino)-5,5-dimethylhexanoic acid;
(S)-2-(benzylamino)-5,5-dimethylhexanoic acid;
(S)-5,5-dimethyl-2-(((1-methyl-1H-indol-4-yl)methyl)amino)hexanoic acid;
(S)-2-(benzhydrylamino)-5,5-dimethylhexanoic acid;
(S)-5,5-dimethyl-2-(((1-methyl-1H-indol-4-yl)methyl)amino)hexanoic acid;
(S)-5,5-dimethyl-2-(((R)-1-phenylethyl)amino)hexanoic acid;
(S)-5,5-dimethyl-2-(((S)-1-phenylethyl)amino)hexanoic acid;
(S)-5,5-dimethyl-2-(((S)-2,2,2-trifluoro-1-phenylethyl)amino)hexanoic acid;
(S)-5,5-dimethyl-2-(((R)-2,2,2-trifluoro-1-phenylethyl)amino)hexanoic acid;
(2S)-5,5-dimethyl-2-{[(3-methylisoquinolin-8-yl)methyl]amino}hexanoic acid;
(2S)-2-{[(3-methoxyphenyl)methyl]amino}-5,5-dimethylhexanoic acid;
(2S)-2-{[(isoquinolin-8-yl)methyl]amino}-5,5-dimethylhexanoic acid;
(2S)-5,5-dimethyl-2-({[2-(trifluoromethoxy)phenyl]methyl}amino)hexanoic acid;
(2S)-2-{[(2-fluorophenyl)methyl]amino}-5,5-dimethylhexanoic acid;
(2S)-2-{[(2,6-difluorophenyl)methyl]amino}-5,5-dimethylhexanoic acid;
(2S)-5,5-dimethyl-2-({[2-(trifluoromethyl)phenyl]methyl}amino)hexanoic acid;
(2S)-2-{[(1S)-2,2-difluoro-1-phenylethyl]amino}-5,5-dimethylhexanoic acid;
(2S)-2-{[(1R)-2,2-difluoro-1-phenylethyl]amino}-5,5-dimethylhexanoic acid;
(S)-2-(((S)-2,2-difluoro-1-(3-methoxyphenyl)ethyl)amino)-5,5-dimethylhexanoic acid;
(S)-2-(((R)-2,2-difluoro-1-(3-methoxyphenyl)ethyl)amino)-5,5-dimethylhexanoic acid;
(S)-5,5-dimethyl-2-(((R)-2,2,2-trifluoro-1-(3-methoxyphenyl)ethyl)amino)hexanoic acid;
(S)-5,5-dimethyl-2-(((S)-2,2,2-trifluoro-1-(3-methoxyphenyl)ethyl)amino)hexanoic acid;
(S)-2-((3-(hydroxymethyl)benzyl)amino)-5,5-dimethylhexanoic acid;
(S)-2-((2,3-dimethoxybenzyl)amino)-5,5-dimethylhexanoic acid;
(S)-2-((3,5-dimethoxybenzyl)amino)-5,5-dimethylhexanoic acid;
(S)-2-((2,5-dimethoxybenzyl)amino)-5,5-dimethylhexanoic acid;
(2S)-2-{[(3-fluoro-5-methoxyphenyl)methyl]amino}-5,5-dimethylhexanoic acid;
(2S)-2-{[(3-chloro-5-methoxyphenyl)methyl]amino}-5,5-dimethylhexanoic acid;
(2S)-2-{[(3-bromo-5-methoxyphenyl)methyl]amino}-5,5-dimethylhexanoic acid;
(2S)-2-{[(3,5-dichlorophenyl)methyl]amino}-5,5-dimethylhexanoic acid;
(2S)-2-{[(3-methoxy-4-methylphenyl)methyl]amino}-5,5-dimethylhexanoic acid;
(2S)-2-{[(2-fluoro-3-methoxyphenyl)methyl]amino}-5,5-dimethylhexanoic acid;
(2S)-5,5-dimethyl-2-{[(quinolin-3-yl)methyl]amino}hexanoic acid;
(2S)-5,5-dimethyl-2-{[(quinolin-2-yl)methyl]amino}hexanoic acid;
(2S)-2-{[(3-fluoro-4-methoxyphenyl)methyl]amino}-5,5-dimethylhexanoic acid;
(2S)-2-{[(3,4-dimethoxyphenyl)methyl]amino}-5,5-dimethylhexanoic acid;
(2S)-5,5-dimethyl-2-{[(5,6,7,8-tetrahydronaphthalen-1-yl)methyl]amino}hexanoic acid;
(2S)-2-{[(3,4-dihydro-2H-1-benzopyran-6-yl)methyl]amino}-5,5-dimethylhexanoic acid;
(2S)-2-{[(2,3-dihydro-1,4-benzodioxin-6-yl)methyl]amino}-5,5-dimethylhexanoic acid;
(2S)-5,5-dimethyl-2-{[(quinoxalin-6-yl)methyl]amino}hexanoic acid;
(2S)-5,5-dimethyl-2-[({1H-pyrrolo[2,3-b]pyridin-5-yl}methyl)amino]hexanoic acid;
(2S)-5,5-dimethyl-2-[({1H-pyrrolo[2,3-b]pyridin-4-yl}methyl)amino]hexanoic acid;
(2S)-2-{[(2H-1,3-benzodioxol-4-yl)methyl]amino}-5,5-dimethylhexanoic acid;
(2S)-5,5-dimethyl-2-{[(quinolin-6-yl)methyl]amino}hexanoic acid;
(2S)-5,5-dimethyl-2-{[(quinolin-8-yl)methyl]amino}hexanoic acid;
(2S)-5,5-dimethyl-2-{[(quinolin-5-yl)methyl]amino}hexanoic acid;
(2S)-2-{[(2-methoxynaphthalen-1-yl)methyl]amino}-5,5-dimethylhexanoic acid;
(2S)-2-{[(1H-indol-2-yl)methyl]amino}-5,5-dimethylhexanoic acid;
(2S)-2-{[(1,3-benzothiazol-5-yl)methyl]amino}-5,5-dimethylhexanoic acid;
(2S)-5,5-dimethyl-2-{[(1-methyl-1H-pyrazol-5-yl)methyl]amino}hexanoic acid;
(2S)-2-{[(1,3-benzothiazol-6-yl)methyl]amino}-5,5-dimethylhexanoic acid;
(2S)-5,5-dimethyl-2-{[(1-methyl-1H-indazol-6-yl)methyl]amino}hexanoic acid;
(2S)-5,5-dimethyl-2-{[(pyrimidin-5-yl)methyl]amino}hexanoic acid;
(2S)-5,5-dimethyl-2-({[2-(pyridin-4-yl)phenyl]methyl}amino)hexanoic acid;
(2S)-2-({[3-(1H-imidazol-1-yl)phenyl]methyl}amino)-5,5-dimethylhexanoic acid;
(2S)-5,5-dimethyl-2-{[(pyridin-4-yl)methyl]amino}hexanoic acid;
(2S)-2-({[2-(hydroxymethyl)phenyl]methyl}amino)-5,5-dimethylhexanoic acid;
(2S)-5,5-dimethyl-2-{[(1,5-naphthyridin-3-yl)methyl]amino}hexanoic acid;
(2S)-2-{[(1S)-1-(3,4-dimethoxyphenyl)-2,2-difluoroethyl]amino}-5,5-dimethylhexanoic acid;
(2S)-2-{[(1,7-dimethyl-1H-indol-4-yl)methyl]amino}-5,5-dimethylhexanoic acid;
(2S)-5,5-dimethyl-2-{[(1-methyl-1H-indazol-4-yl)methyl]amino}hexanoic acid;
(2S)-5,5-dimethyl-2-{[(1R)-1-(1-methyl-1H-indol-4-yl)ethyl]amino}hexanoic acid;
(2S)-2-{[(6-methoxynaphthalen-2-yl)methyl]amino}-5,5-dimethylhexanoic acid;
(2S)-2-{[(1S)-1-(3,4-dimethoxyphenyl)-2,2,2-trifluoroethyl]amino}-5,5-dimethylhexanoic acid;
(2S)-2-{[(1R)-1-(3,4-dimethoxyphenyl)-2,2-difluoroethyl]amino}-5,5-dimethylhexanoic acid;
(2S)-5,5-dimethyl-2-{[(1-methyl-1H-indol-7-yl)methyl]amino}hexanoic acid;
(2S)-2-{[(3,4-dimethylphenyl)methyl]amino}-5,5-dimethylhexanoic acid;
(2S)-2-{[(1S)-1-(4-methoxy-3-methylphenyl)ethyl]amino}-5,5-dimethylhexanoic acid;
(2S)-2-{[(1R)-1-(3,4-dimethoxyphenyl)-2,2,2-trifluoroethyl]amino}-5,5-dimethylhexanoic acid;
(2S)-5,5-dimethyl-2-{[(1-methyl-1H-1,3-benzodiazol-5-yl)methyl]amino}hexanoic acid;
(2S)-5,5-dimethyl-2-{[(1-methyl-1H-1,3-benzodiazol-4-yl)methyl]amino}hexanoic acid;
(2S)-2-{[(1S)-1-(3,4-dimethoxyphenyl)ethyl]amino}-5,5-dimethylhexanoic acid;
(2S)-5,5-dimethyl-2-{[(2-methylpyrimidin-5-yl)methyl]amino}hexanoic acid;
(2S)-5,5-dimethyl-2-{[(2-methyl-1,3-benzothiazol-5-yl)methyl]amino}hexanoic acid;
(2S)-2-{[(3-chloro-4-methylphenyl)methyl]amino}-5,5-dimethylhexanoic acid;
(2S)-2-{[(3-hydroxy-4-methoxyphenyl)methyl]amino}-5,5-dimethylhexanoic acid;
(2S)-5,5-dimethyl-2-{[(1-methyl-1H-1,2,3-benzotriazol-5-yl)methyl]amino}hexanoic acid;
(2S)-5,5-dimethyl-2-{[(pyrimidin-4-yl)methyl]amino}hexanoic acid;
(2S)-5,5-dimethyl-2-{[(1S)-1-(1-methyl-1H-indol-4-yl)ethyl]amino}hexanoic acid;
(2S)-2-{[(3-acetylphenyl)methyl]amino}-5,5-dimethylhexanoic acid;
(2S)-2-{[(1-ethyl-1H-indol-4-yl)methyl]amino}-5,5-dimethylhexanoic acid;
(2S)-2-{[(1-benzofuran-5-yl)methyl]amino}-5,5-dimethylhexanoic acid;
(2S)-2-{[(1S)-1-(2-methoxypyridin-4-yl)ethyl]amino}-5,5-dimethylhexanoic acid;
(2S)-2-{[(1R)-1-(4-methoxy-3-methylphenyl)ethyl]amino}-5,5-dimethylhexanoic acid;
(2S)-2-{[(3,4-dichlorophenyl)methyl]amino}-5,5-dimethylhexanoic acid;
(2S)-2-{[(5-bromopyridin-3-yl)methyl]amino}-5,5-dimethylhexanoic acid;
(2S)-5,5-dimethyl-2-[({1-methyl-1H-pyrrolo[2,3-b]pyridin-5-yl}methyl)amino]hexanoic acid;
(2S)-2-{[(5-methoxypyridin-3-yl)methyl]amino}-5,5-dimethylhexanoic acid;
(2S)-2-{[(1H-indol-4-yl)methyl]amino}-5,5-dimethylhexanoic acid;
(2S)-2-{[(isoquinolin-4-yl)methyl]amino}-5,5-dimethylhexanoic acid;
(2S)-5,5-dimethyl-2-{[(1R)-1-(pyrimidin-5-yl)ethyl]amino}hexanoic acid;
(2S)-2-{[(4-methoxyphenyl)methyl]amino}-5,5-dimethylhexanoic acid;
(2S)-5,5-dimethyl-2-{[(5-methylpyridin-3-yl)methyl]amino}hexanoic acid;
(2S)-2-{[(2,3-dimethylphenyl)methyl]amino}-5,5-dimethylhexanoic acid;
(2S)-5,5-dimethyl-2-{[(1S)-1-(pyrimidin-5-yl)ethyl]amino}hexanoic acid;
(2S)-2-{[(2H-indazol-4-yl)methyl]amino}-5,5-dimethylhexanoic acid;
(2S)-5,5-dimethyl-2-{[(6-methylpyridin-3-yl)methyl]amino}hexanoic acid;
(2S)-2-{[(2-chloro-3-fluoropyridin-4-yl)methyl]amino}-5,5-dimethylhexanoic acid;
(2S)-2-{[(isoquinolin-5-yl)methyl]amino}-5,5-dimethylhexanoic acid;
(2S)-2-{[(4-chlorophenyl)methyl]amino}-5,5-dimethylhexanoic acid;
(2S)-2-{[(2-methoxypyridin-4-yl)methyl]amino}-5,5-dimethylhexanoic acid;
(2S)-2-{[(2-fluoro-5-methoxyphenyl)methyl]amino}-5,5-dimethylhexanoic acid;
(2S)-5,5-dimethyl-2-{[(2-methylpyridin-4-yl)methyl]amino}hexanoic acid;
(2S)-5,5-dimethyl-2-{[(1-methyl-1H-indol-6-yl)methyl]amino}hexanoic acid;
(2S)-2-{[(1R)-1-(3,4-dimethoxyphenyl)ethyl]amino}-5,5-dimethylhexanoic acid;
(2S)-5,5-dimethyl-2-{[(3-methylpyridin-4-yl)methyl]amino}hexanoic acid;
(2S)-2-{[(3-methoxy-5-methylphenyl)methyl]amino}-5,5-dimethylhexanoic acid;
(2S)-2-{[(3-cyanophenyl)methyl]amino}-5,5-dimethylhexanoic acid;
(2S)-2-{[(4-methoxynaphthalen-1-yl)methyl]amino}-5,5-dimethylhexanoic acid;
(2S)-2-{[(4-fluoro-3-methoxyphenyl)methyl]amino}-5,5-dimethylhexanoic acid;
(2S)-5,5-dimethyl-2-{[(pyridin-3-yl)methyl]amino}hexanoic acid; and
(2S)-2-{[(3-methoxy-2-methylphenyl)methyl]amino}-5,5-dimethylhexanoic acid.

13. The compound according claim 3, wherein R⁵ is selected from the group consisting of:
(i) phenyl, naphthyl, 5- or 6-membered monocyclic heteroaryl, and 9- or 10-membered fused bicyclic heteroaryl, each of which is optionally substituted with one or more substituents independently selected from the group consisting of halo, —OH, (C₁-C₂)alkyl, (C₁-C₂)alkoxy, (C₁-C₂)hydroxyalkyl, (C₁-C₂)haloalkyl, (C₁-C₂)haloalkoxy, acetyl, cyano, imidazolyl, and pyridyl, wherein no more than 2 ring atoms in the 5- or 6-membered monocyclic heteroaryl group are heteroatoms and no more than 3 ring atoms in the 9- or 10-membered fused bicyclic heteroaryl group are heteroatoms; and

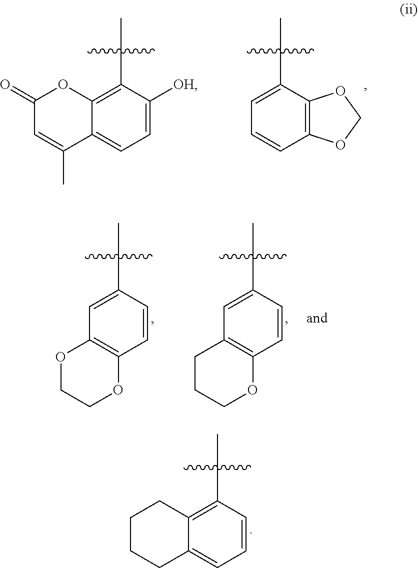 [SMILES: *c1c(O)ccc2c(C)cc(=O)oc12 ; *c1cccc2c1OCO2 ; *c1ccc2c(c1)OCCO2 ; *c1ccc2c(c1)CCCO2 ; *c1cccc2c1CCCC2]

14. The compound according to claim 13, wherein R⁵ is selected from the group consisting of:
(i) phenyl, pyridyl, pyrimidinyl, pyrazolyl, quinolinyl, isoquinolinyl, indolyl, azaindolyl, quinoxalinyl, benzothiazolyl, indazolyl, naphthyridinyl, naphthyl, benzimidazolyl, benzotriazolyl, and benzofuranyl, each of which is optionally substituted with one or more substituents independently selected from the group consisting of halo, —OH, (C₁-C₂)alkyl, (C₁-C₂)alkoxy, (C₁-C₂)hydroxyalkyl, (C₁-C₂)haloalkyl, (C₁-C₂)haloalkoxy, acetyl, cyano, imidazolyl, and pyridyl; and

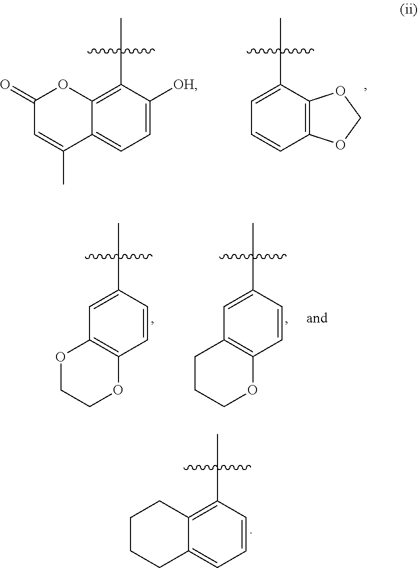 [SMILES: *c1c(O)ccc2c(C)cc(=O)oc12 ; *c1cccc2c1OCO2 ; *c1ccc2c(c1)OCCO2 ; *c1ccc2c(c1)CCCO2 ; *c1cccc2c1CCCC2]

15. The compound according to claim 14, wherein R⁵ is selected from the group consisting of:
(i) phenyl, optionally substituted with one or more substituents independently selected from the group consisting of halo, —OH, (C₁-C₂)alkyl, (C₁-C₂)alkoxy, (C₁-C₂)hydroxyalkyl, (C₁-C₂)haloalkyl, (C₁-C₂)haloalkoxy, acetyl, cyano, imidazolyl, and pyridyl;
(ii) pyridyl, optionally substituted with one or more substituents independently selected from the group consisting of halo, (C₁-C₂)alkyl, (C₁-C₂)alkoxy;
(iii) pyrimidinyl and pyrazolyl, each of which is optionally substituted with one or more substituents independently selected from the group consisting of (C₁-C₂)alkyl;
(iv) quinolinyl, isoquinolinyl, indolyl, azaindolyl, quinoxalinyl, benzothiazolyl, indazolyl, naphthyridinyl, naphthyl, benzimidazolyl, benzotriazolyl, and benzofuranyl, each of which is optionally substituted with one or more substituents selected from the group consisting of (C₁-C₂)alkyl and (C₁-C₂)alkoxy; and

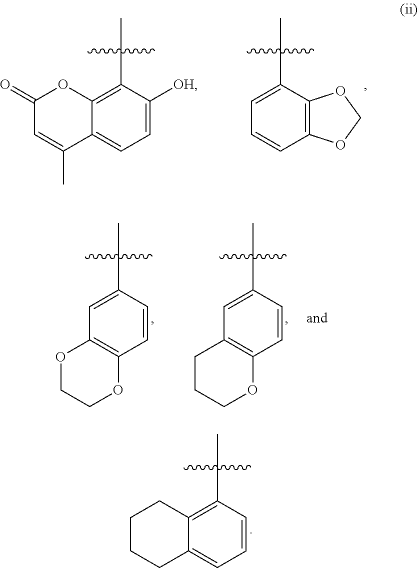 [SMILES: *c1c(O)ccc2c(C)cc(=O)oc12 ; *c1cccc2c1OCO2 ; *c1ccc2c(c1)OCCO2 ; *c1ccc2c(c1)CCCO2 ; *c1cccc2c1CCCC2]

16. The compound according to claim 15, wherein R⁵ is one of the following groups:

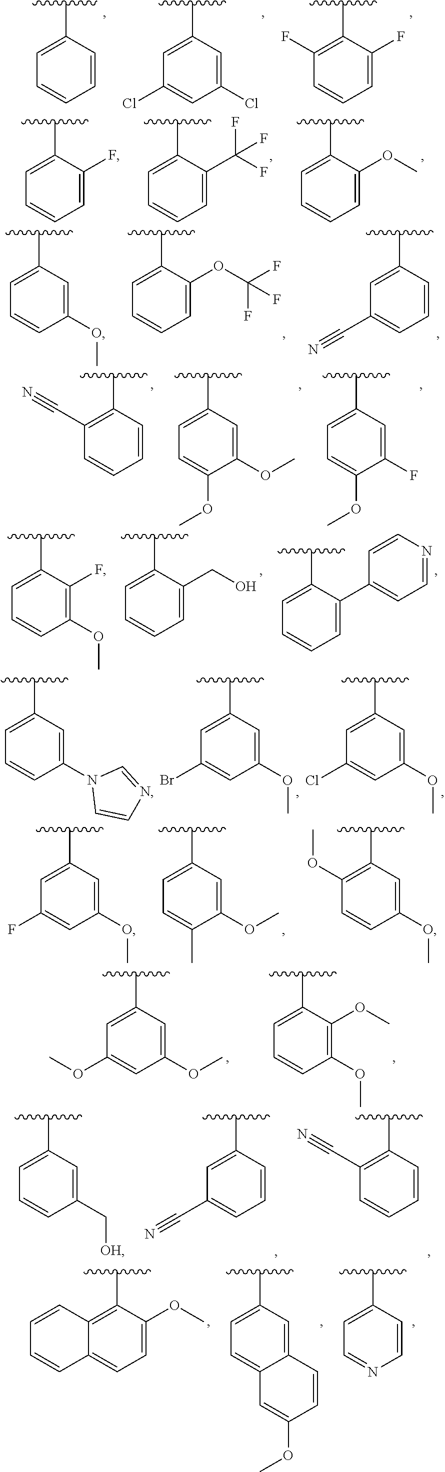

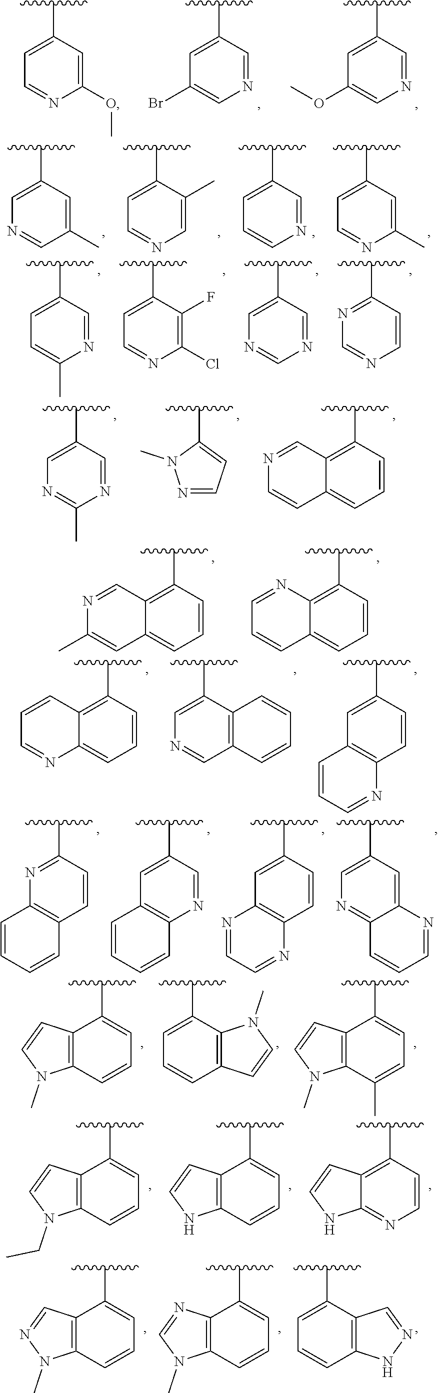

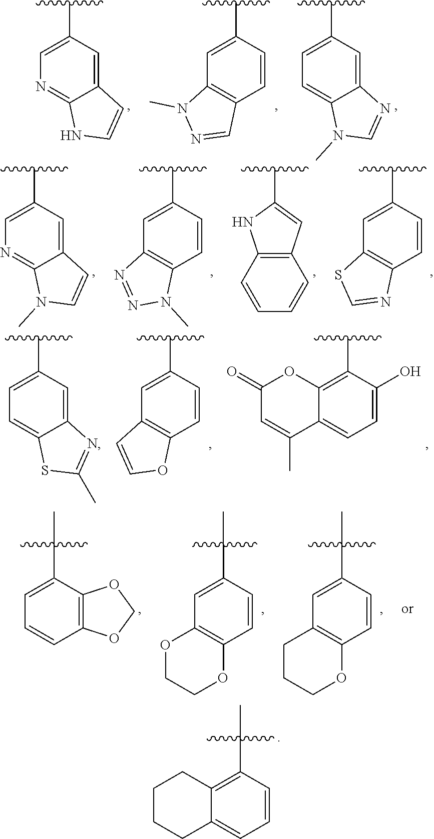

17. A pharmaceutical composition comprising a compound according to claim 1 and a pharmaceutically acceptable carrier, excipient, and/or diluent.

18. A method of therapy comprising administration of the compound of claim 1.

19. A method of treatment or prevention of a neurodegenerative disorder, a psychiatric disorder, an inflammatory disorder, a cancer, pain, diabetes mellitus, diabetic retinopathy, glaucoma, uveitis, cardiovascular disease, kidney disease, psoriasis, hereditary eye conditions, hearing loss or diseases characterized by misfolded tau comprising administration of the compound of claim 3;
preferably wherein the neurodegenerative disorder is selected from motor neuron diseases, Frontotemporal Lobar Degeneration (FTLD), frontotemporal dementia, Alzheimer's disease, Parkinson's disease, Huntington's disease, prion diseases such as Creutzfeldt-Jakob disease (CJD), acute brain injury, spinal cord injury and stroke, preferably wherein the motor neuron disease is selected from amyotrophic lateral sclerosis (ALS), Primary Lateral Sclerosis, and Progressive Muscular Atrophy;
wherein the neurodegenerative disorder is preferably a neurodegenerative disorder characterised by misfolded TAR DNA-binding protein 43, such as amyotrophic lateral sclerosis, Alzheimer's disease, Frontotemporal Lobar Degeneration, or frontotemporal dementia;
wherein the psychiatric disorder is selected from bipolar disorder, major depression, post-traumatic stress disorder, and anxiety disorders.
wherein the inflammatory disorder may be selected from inflammatory diseases and neuroinflammation;
wherein the cancer is selected from breast cancer, lung cancer, ovarian cancer, prostate cancer, thyroid cancer, pancreatic cancer, glioblastoma and colorectal cancer;
wherein the cardiovascular disease is preferably selected from atherosclerosis, cardiomyopathy, heart attack, arrhythmias, heart failure, and ischemic heart disease; and
wherein the hearing loss is selected from noise-induced hearing loss, ototoxicity induced hearing loss, age-induced hearing loss, idiopathic hearing loss, tinnitus and sudden hearing loss.


SEQUENCE LISTING (4 sequences)

SEQ ID NO: 1 - protein, 831 aa, Homo sapiens [sequence omitted]

SEQ ID NO: 2 - protein, 830 aa, Homo sapiens
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 3 - protein, 694 aa, Homo sapiens
MTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 4 - protein, 825 aa, Mus musculus
MERPRGAADGLLRWPLGLLLLLQLLPPAAVGQDRLDAPPPPAPPLLRWAGPVGVSWGLRAAAPGGPVPRAGRWRRGAPAEDQDCGRLPDFIAKLTNNTHQHVFDDLSGSVSLSWVGDSTGVILVLTTFQVPLVIVSFGQSKLYRSEDYGKNFKDITNLINNTFIRTEFGMAIGPENSGKVILTAEVSGGSRGGRVFRSSDFAKNFVQTDLPFHPLTQMMYSPQNSDYLLALSTENGLWVSKNFGEKWEEIHKAVCLAKWGPNNIIFFTTHVNGSCKADLGALELWRTSDLGKTFKTIGVKIYSFGLGGRFLFASVMADKDTTRRIHVSTDQGDTWSMAQLPSVGQEQFYSILAANEDMVFMHVDEPGDTGFGTIFTSDDRGIVYSKSLDRHLYTTTGGETDFTNVTSLRGVYITSTLSEDNSIQSMITFDQGGRWEHLRKPENSKCDATAKNKNECSLHIHASYSISQKLNVPMAPLSEPNAVGIVIAHGSVGDAISVMVPDVYISDDGGYSWAKMLEGPHYYTILDSGGIIVAIEHSNRPINVIKFSTDEGQCWQSYVFTQEPIYFTGLASEPGARSMNISIWGFTESFITRQWVSYTVDFKDILERNCEEDDYTTWLAHSTDPGDYKDGCILGYKEQFLRLRKSSVCQNGRDYVVAKQPSVCPCSLEDFLCDFGYFRPENASECVEQPELKGHELEFCLYGKEEHLTTNGYRKIPGDKCQGGMNPAREVKDLKKKCTSNFLNPTKQNSKSNSVPIILAIVGLMLVTVVAGVLIVKKYVCGGRFLVHRYSVLQQHAEADGVEALDSTSHAKSGYHDDSDEDLLE